(19) United States
(12) Patent Application Publication
(10) Pub. No.: US 2025/0154466 A1
(43) Pub. Date: May 15, 2025

(54) COMBINATION BCL11A ENHANCER EDITING

(71) Applicant: THE CHILDREN'S MEDICAL CENTER CORPORATION, Boston, MA (US)

(72) Inventors: Daniel E. BAUER, Cambridge, MA (US); Jing ZENG, Brookline, MA (US)

(73) Assignee: THE CHILDREN'S MEDICAL CENTER CORPORATION, Boston, MA (US)

(21) Appl. No.: 18/717,370

(22) PCT Filed: Dec. 9, 2022
(86) PCT No.: PCT/US2022/052359

§ 371 Date: Jun. 6, 2024

Related U.S. Application Data
(60) Provisional application No. 63/288,123, filed on Dec. 10, 2021.

(51) Int. Cl. C12N 5/0789; C12N 5/074; C12N 9/22; C12N 15/11; C12N 15/85

(52) U.S. Cl. CPC C12N 5/0647; C12N 5/0696; C12N 9/22; C12N 15/111; C12N 15/85; C07K 2319/09; C12N 2310/20

(57) ABSTRACT

Provided herein are vectors, and compositions thereof, comprising at least two guide RNAs (gRNAs) that targets and hybridizes to a target sequence on a DNA molecule, wherein each of the at least two gRNAs hybridizes at least two genomic DNA locations selected from human chromosome 2 at location 60725424 to 60725688 according to GRCh37/hg19 human genome reference build (+55 functional region); human chromosome 2 at location 60722238 to 60722466 GRCh37/hg19 human genome reference build (+58 functional region); or human chromosome 2 at location 60718042 to 60718186 according to GRCh37/hg19 human genome reference build (+62 functional region). Methods for the use of such vectors are further provided.

### CROSS-REFERENCE TO RELATED APPLICATIONS

This application claims benefit under 35 U.S.C. § 119(e) of U.S. Provisional Application No. 63/288,123 filed Dec. 10, 2021, the contents of which are incorporated herein by reference in their entirety.

### GOVERNMENT SUPPORT

This invention was made with government support under Grant Number HL053749, awarded by the National Institutes of Health. The government has certain rights in the invention.

### TECHNICAL FIELD

The present invention relates to a novel fetal hemoglobin modulating activity for BCL11A and methods for uses therein.

### BACKGROUND

Normal adult hemoglobin comprises four globin proteins, two of which are alpha (α) proteins and two of which are beta (β) proteins. During mammalian fetal development, particularly in humans, the fetus produces fetal hemoglobin, which comprises two gamma (γ)-globin proteins instead of the two β-globin proteins. During the neonatal period, a globin switch occurs, referred to as the “fetal switch”, at which point, erythroid precursors switch from making predominantly γ-globin to making predominantly β-globin. The developmental switch from production of predominantly fetal hemoglobin or HbF (α₂γ₂) to production of adult hemoglobin or HbA (α₂β₂) begins at about 28 to 34 weeks of gestation and continues shortly after birth until HbA becomes predominant. This switch results primarily from decreased transcription of the gamma-globin genes and increased transcription of beta-globin genes. On average, the blood of a normal adult contains less than 1% HbF, though residual HbF levels have a variance of over 20 fold in healthy adults and are genetically controlled.

Hemoglobinopathies encompass a number of anemias of genetic origin in which there is a decreased production and/or increased destruction (hemolysis) of red blood cells (RBCs). These also include genetic defects that result in the production of abnormal hemoglobins with a concomitant impaired ability to maintain oxygen concentration. Some such disorders involve the failure to produce normal β-globin in sufficient amounts, while others involve the failure to produce normal β-globin entirely. These disorders associated with the β-globin protein are referred to generally as β-hemoglobinopathies. For example, β-thalassemias result from a partial or complete defect in the expression of the β-globin gene, leading to deficient or absent HbA. Sickle cell anemia results from a point mutation in the β-globin structural gene, leading to the production of an abnormal (sickle) hemoglobin (HbS). HbS is prone to polymerization, particularly under deoxygenated conditions. HbS RBCs are more fragile than normal RBCs and undergo hemolysis more readily, leading eventually to anemia.

Work described herein sought to optimize conditions of the CRISPR-Cas gene editing system in patient-derived HSCs as a nearly complete reaction lacking detectable genotoxicity or deleterious impact on stem cell function. Described herein are CRISPR enzymes in combination with at least two guide RNAs (gRNAs), where the number of gRNAs increases its gene-editing efficacy as compared to the use of a single guide RNA (gRNA), for example in a quiescent cell.

### SUMMARY

One aspect provided herein describes a vector comprising at least two guide RNAs (gRNAs) that targets and hybridizes to a target sequence on a DNA molecule, wherein each of the at least two gRNAs hybridizes at least two genomic DNA locations selected from human chromosome 2 at location 60725424 to 60725688 according to GRCh37/hg19 human genome reference build (+55 functional region); human chromosome 2 at location 60722238 to 60722466 GRCh37/hg19 human genome reference build (+58 functional region); or human chromosome 2 at location 60718042 to 60718186 according to GRCh37/hg19 human genome reference build (+62 functional region).

In one embodiment of any aspect described herein, the gRNA has a selected from the group consisting of #1617: CUAACAGUUGCUUUUAUCAC (SEQ ID NO: 1), #1618: UUGCUUUUAUCACAGGCUCC (SEQ ID NO: 2), #1449: GUGCUUGGUCGGCACUGAUA (SEQ ID NO: 3), and #1450: UGCUUGGUCGGCACUGAUAG (SEQ ID NO: 4).

In one embodiment of any aspect described herein, the vector further comprises a DNA endonuclease enzyme.

Another aspect provided herein describes a ribonucleoprotein (RNP) complex comprising a DNA endonuclease enzyme and at least two guide RNAs (gRNAs) that targets and hybridizes to a target sequence on a DNA molecule, wherein each of the at least two gRNAs hybridizes at least two genomic DNA locations selected from human chromosome 2 at location 60725424 to 60725688 according to GRCh37/hg19 human genome reference build (+55 functional region); human chromosome 2 at location 60722238 to 60722466 GRCh37/hg19 human genome reference build (+58 functional region); or human chromosome 2 at location 60718042 to 60718186 according to GRCh37/hg19 human genome reference build (+62 functional region).

In one embodiment of any aspect described herein, the at least two gRNAs hybridize to the same genomic DNA location.

In one embodiment of any aspect described herein, the at least two gRNAs hybridize to different genomic DNA locations.

In one embodiment of any aspect described herein, the RNP has one gRNA that hybridizes to the +55 functional region and one gRNA that hybridizes to the +58 functional region.

In one embodiment of any aspect described herein, the RNP has one gRNA that hybridizes to the +55 functional region and one gRNA that hybridizes to the +62 functional region.

In one embodiment of any aspect described herein, the RNP has one gRNA that hybridizes to the +58 functional region and one gRNA that hybridizes to the +62 functional region.

In one embodiment of any aspect described herein, the RNP has one gRNA that hybridizes to the +55 functional region, one gRNA that hybridizes to the +58 functional region, one gRNA that hybridizes to the +62 functional region

In one embodiment of any aspect described herein, the gRNA is selected from the group consisting of #1617: CUAACAGUUGCUUUUAUCAC (SEQ ID NO: 1), #1618: UUGCUUUUAUCACAGGCUCC (SEQ ID NO: 2), #1449: GUGCUUGGUCGGCACUGAUA (SEQ ID NO: 3), and #1450: UGCUUGGUCGGCACUGAUAG (SEQ ID NO: 4).

In one embodiment of any aspect described herein, the DNA endonuclease comprises at least one nuclear localization signal (NLS) sequence fused at or near its amino terminus.

In one embodiment of any aspect described herein, the DNA endonuclease further comprises at least one NLS sequence fused at or near its carboxy terminus.

In one embodiment of any aspect described herein, the DNA endonuclease has one NLS sequence fused at or near its amino terminus, and two nuclear localization signal sequences fused at or near its carboxy terminus.

In one embodiment of any aspect described herein, the NLS sequence is selected from the group consisting of: SV40 large T-antigen, nucleoplasmin, c-Myc, c-Myc-like, and hRNPA1.

In one embodiment of any aspect described herein, the at least one nuclear localization signal sequences are identical.

In one embodiment of any aspect described herein, the at least one nuclear localization signal sequences are different.

In one embodiment of any aspect described herein, the DNA endonuclease has a c-Myc-like nuclear localization signal sequence fused to its amino terminus and an SV40 nuclear localization signal sequence and nucleplasmin bipartate nuclear localization signal sequence fused to its carboxyl terminus.

In one embodiment of any aspect described herein, the DNA endonuclease is selected from the group consisting of a Zinc Finger Nuclease (ZFN), a Transcription Activator-Like Effector Nuclease (TALENs), and a CRISPR-associated protein (Cas) nuclease.

In one embodiment of any aspect described herein, the CRISPR enzyme is a type II CRISPR system enzyme.

In one embodiment of any aspect described herein, the CRISPR enzyme is a Cas protein.

In one embodiment of any aspect described herein, the Cas protein is selected from the group consisting of: Cpf1, C2c1, C2c3, Cas12a, Cas12b, Cas12c, Cas12d, Cas12e, Cas13a, Cas13b, and Cas13c. Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c.

In one embodiment of any aspect described herein, the RNP is for the use of altering the expression of at least one gene product.

Another aspect provided herein describes a vector comprising the any of the RNPs described herein.

Another aspect provided herein describes a composition comprising the any of the vectors or RNPs described herein.

Another aspect provided herein describes a pharmaceutical composition of comprising any of the vectors, RNPs, or vectors described herein.

Another aspect provided herein describes a method for producing a progenitor cell having decreased BCL11A mRNA or protein expression, the method comprising contacting an isolated progenitor cell with any of the vectors, RNPs, or compositions described herein.

Another aspect provided herein describes a method for producing an isolated genetic engineered human cell having at least one genetic modification comprising contacting an isolated cell with an effective amount of any of the vectors, RNPs, or compositions described herein, causing at least one genetic modification therein.

In one embodiment of any aspect described herein, the isolated progenitor cell or isolated cell is a hematopoietic progenitor cell. In one embodiment of any aspect described herein, the isolated progenitor cell or isolated cell is a hematopoietic stem cell.

In one embodiment of any aspect described herein, the hematopoietic progenitor is a cell of the erythroid lineage.

In one embodiment of any aspect described herein, the isolated progenitor cell or isolated cell is an induced pluripotent stem cell.

In one embodiment of any aspect described herein, the hematopoietic progenitor cell is contacted ex vivo or in vitro.

In one embodiment of any aspect described herein, the at least one genetic modification is an inversion, an insertion or a deletion.

Another aspect provided herein describes a composition comprising progenitor cells obtained by the any method described herein.

Another aspect provided herein describes a composition comprising isolated genetic engineered human cells obtained by any method described herein.

Another aspect provided herein describes a method of increasing fetal hemoglobin levels in a cell, the method comprising the steps of: contacting an isolated cell with an effective amount of a composition comprising any of the vectors, RNPs, or compositions described herein, whereby fetal hemoglobin expression is increased in said cell, or its progeny, relative to said cell prior to said contacting.

In one embodiment of any aspect described herein, the isolated cell is a hematopoietic progenitor cell.

In one embodiment of any aspect described herein, the hematopoietic progenitor cell is a cell of the erythroid lineage.

In one embodiment of any aspect described herein, the isolated cell is an induced pluripotent stem cell.

In one embodiment of any aspect described herein, the hematopoietic progenitor cell is contacted ex vivo or in vitro.

In one embodiment of any aspect described herein, the at least one genetic modification is an inversion, an insertion or a deletion.

Another aspect provided herein describes a method for increasing fetal hemoglobin levels in a mammal in need thereof, the method comprising the steps of contacting an isolated hematopoietic progenitor cell in said mammal with an effective amount of a composition comprising any of the vectors, RNPs, or compositions described herein, causing at least one genetic modification therein, whereby fetal hemoglobin expression is increased in said mammal, relative to expression prior to said contacting.

Another aspect provided herein describes a method for increasing fetal hemoglobin levels in a mammal in need thereof, the method comprising transplanting an isolated genetic engineered human cell described herein or any composition described herein into the mammal.

### Definitions

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

As used herein, the term “genetic engineered cell” refers to a cell that comprises at least one genetic modification, as that term is used herein.

As used herein, the term “genetic modification” refers to a disruption at the genomic level resulting in a decrease in BCL11A expression or activity in a cell. Exemplary genetic modifications can include deletions, insertions, inversions, frame shift mutations, point mutations, exon removal, removal of one or more DNAse 1-hypersensitive sites (DHS) (e.g., 2, 3, 4 or more DHS regions), etc.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given vector, composition, or RNP complex described herein) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. Where applicable, a decrease can be preferably down to a level accepted as within the range of normal for an individual without a given disorder.

By “decreased BCL11A expression” is meant that the amount of expression of BCL11A is at least 5% lower in a cell or cell population treated with a DNA-targeting endonuclease, than a comparable, control cell or cell population, wherein no DNA-targeting endonuclease is present. It is preferred that the percentage of BCL11A expression in a treated population is at least 10% lower, at least 20% lower, at least 30% lower, at least 40% lower, at least 50% lower, at least 60% lower, at least 70% lower, at least 80% lower, at least 90% lower, at least 1-fold lower, at least 2-fold lower, at least 5-fold lower, at least 10 fold lower, at least 100 fold lower, at least 1000-fold lower, or more than a comparable control treated population in which no DNA-targeting endonuclease is added.

By “decreased BCL11A activity” is meant that the amount of functional activity of BCL11A is at least 5% lower in a cell or cell population treated with the methods described herein, than a comparable, control cell or population, wherein no DNA-targeting endonuclease is present. It is preferred that the percentage of BCL11A activity in a BCL11A-inhibitor treated population is at least 10% lower, at least 20% lower, at least 30% lower, at least 40% lower, at least 50% lower, at least 60% lower, at least 70% lower, at least 80% lower, at least 90% lower, at least 1-fold lower, at least 2-fold lower, at least 5-fold lower, at least 10 fold lower, at least 100 fold lower, at least 1000-fold lower, or more than a comparable control treated population in which no DNA-targeting endonuclease is added. At a minimum, BCL11A activity can be assayed by determining the amount of BCL11A expression at the protein or mRNA levels, using techniques standard in the art. Alternatively, or in addition, BCL11A activity can be determined using a reporter construct, wherein the reporter construct is sensitive to BCL11A activity. The γ-globin locus sequence is recognizable by the nucleic acid-binding motif of the BCL11A construct.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

In one embodiment, as used herein, the term “DNA targeting endonuclease” refers to an endonuclease that generates a double-stranded break at a desired position in the genome (e.g., chromosome 2 location 60,716,189-60,728,612) without producing undesired off-target double-stranded breaks. The DNA targeting endonuclease can be a naturally occurring endonuclease (e.g., a bacterial meganuclease) or it can be artificially generated (e.g., engineered meganucleases, TALENs, or ZFNs, among others).

In another embodiment, as used herein, the term “DNA targeting endonuclease” refers to an endonuclease that generates a single-stranded break or a “nick” or break on one strand of the DNA phosphate sugar backbone at a desired position in the genome (e.g., chromosome 2 location 60725424 to 60725688 (+55 functional region), at location 60722238 to 60722466 (+58 functional region), and/or at location 60718042 to 60718186 (+62 functional region)) without producing undesired off-target DNA stranded breaks.

As used herein, a “subject” means a human or animal. Usually the animal is a vertebrate such as a primate, rodent, domestic animal or game animal. Primates include, for example, chimpanzees, cynomologous monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of disease e.g., hemoglobinopathies or cancer. A subject can be male or female.

A subject can be one who has been previously diagnosed with or identified as suffering from or having a condition in need of treatment (e.g. a hemoglobinopathy) or one or more complications related to such a condition, and optionally, have already undergone treatment for the condition or the one or more complications related to the condition. Alternatively, a subject can also be one who has not been previously diagnosed as having such condition or related complications. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, “vector” refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “viral vector” refers to a nucleic acid vector construct that includes at least one element of viral origin and has the capacity to be packaged into a viral particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

Within an expression vector, “operably linked” is intended to mean that the nucleotide sequence of interest is linked to the regulatory sequence(s) in a manner which allows for expression of the nucleotide sequence (e.g., in an in vitro transcription/translation system or in a target cell when the vector is introduced into the target cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, CA (1990). Regulatory sequences include those which direct constitutive expression of a nucleotide sequence in many types of host cell and those which direct expression of the nucleotide sequence only in certain host cells (e.g., tissue-specific regulatory sequences). Furthermore, the DNA-targeting endonuclease can be delivered by way of a vector comprising a regulatory sequence to direct synthesis of the DNA-targeting endonuclease at specific intervals, or over a specific time period. It will be appreciated by those skilled in the art that the design of the expression vector can depend on such factors as the choice of the target cell, the level of expression desired, and the like.

In one embodiment, the term “engineered” and its grammatical equivalents as used herein can refer to one or more human-designed alterations of a nucleic acid, e.g., the nucleic acid within an organism's genome. In another embodiment, engineered can refer to alterations, additions, and/or deletion of genes. An “engineered cell” can refer to a cell with an added, deleted and/or altered gene. The term “cell” or “engineered cell” and their grammatical equivalents as used herein can refer to a cell of human or non-human animal origin.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of ordinary skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physicochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. ligand-mediated receptor activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, a polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to an assay known in the art or described below herein. For example, a functional fragment described herein would retain at least 50% of the CRISPR enzyme function. One skilled in the art can assess the function of a CRISPR enzyme using standard techniques, for example those described herein below. A functional fragment can comprise conservative substitutions of the sequences disclosed herein.

In some embodiments, a polypeptide described herein can be a variant of a polypeptide or molecule as described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity of the non-variant polypeptide. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of a polypeptide also can be substituted, generally with serine, to improve the oxidative stability of the molecule and prevent aberrant crosslinking. Conversely, cysteine bond(s) can be added to a polypeptide to improve its stability or facilitate oligomerization.

As used herein, the term “DNA” is defined as deoxyribonucleic acid. The term “polynucleotide” is used herein interchangeably with “nucleic acid” to indicate a polymer of nucleosides. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The term “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide.” Exemplary modifications include glycosylation and palmitoylation. Polypeptides can be purified from natural sources, produced using recombinant DNA technology or synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

As used herein, the term “effective amount of a composition” refers to an amount of composition that yields sufficient endonuclease activity to generate a double-stranded break in the desired location of the genome. In one embodiment, the effective amount of a DNA-targeting endonuclease generates a double-stranded break at the desired genetic locus in at least 20% of the cells in a population contacted with the composition (e.g., at least 30%, at least 40%, at least 50%, at least 60%, at least 70%, at least 80%, at least 90%, at least 95%, at least 99%, or even 100% of the cells in the population comprise a genetic modification produced by the DNA-targeting endonuclease composition).

The term “isolated cell” as used herein refers to a cell that has been removed from an organism in which it was originally found, or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched. In some embodiments, the isolated population is an isolated population of human hematopoietic progenitor cells, e.g., a substantially pure population of human hematopoietic progenitor cells as compared to a heterogeneous population of cells comprising human hematopoietic progenitor cells and cells from which the human hematopoietic progenitor cells were derived.

The term “substantially pure,” with respect to a particular cell population, refers to a population of cells that is at least about 75%, preferably at least about 85%, more preferably at least about 90%, and most preferably at least about 95% pure, with respect to the cells making up a total cell population. That is, the terms “substantially pure” or “essentially purified,” with regard to a population of hematopoietic progenitor cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not hematopoietic progenitor cells as defined by the terms herein.

In one embodiment, as used herein, “prevent,” and similar words such as “prevented,” “preventing” etc., indicate an approach for preventing, inhibiting, or reducing the likelihood of the occurrence or recurrence of, a disease or condition. In another embodiment, the term refers to delaying the onset or recurrence of a disease or condition or delaying the occurrence or recurrence of the symptoms of a disease or condition. In another embodiment, as used herein, “prevention” and similar words includes reducing the intensity, effect, symptoms and/or burden of a disease or condition prior to onset or recurrence of the disease or condition.

As used herein, the term “treating” includes reducing or alleviating at least one adverse effect or symptom of a condition, disease or disorder. For example, the term “treating” and “treatment” refers to administering to a subject an effective amount of a composition, e.g., an effective amount of a composition comprising a population of hematopoietic progenitor cells so that the subject has a reduction in at least one symptom of the disease or an improvement in the disease, for example, beneficial or desired clinical results. For purposes of this disclosure, beneficial or desired clinical results include, but are not limited to, alleviation of one or more symptoms, diminishment of extent of disease, disease stabilization (e.g., not worsening), delay or slowing of disease progression, amelioration or palliation of the disease state, and remission (whether partial or total), whether detectable or undetectable. In some embodiments, treating can refer to prolonging survival as compared to expected survival if not receiving treatment. Thus, one of skill in the art realizes that a treatment can improve the disease condition, but may not be a complete cure for the disease. In some embodiments, treatment can include prophylaxis. However, in alternative embodiments, treatment does not include prophylaxis.

As used herein, the term “administering,” refers to the placement of a therapeutic or pharmaceutical composition as disclosed herein into a subject by a method or route which results in at least partial delivery of the agent at a desired site. Pharmaceutical compositions comprising agents as disclosed herein can be administered by any appropriate route which results in an effective treatment in the subject.

As used herein, the term “pharmaceutical composition” refers to the active agent in combination with a pharmaceutically acceptable carrier e.g. a carrier commonly used in the pharmaceutical industry.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. As used herein, “prevention” or “preventing,” when used in reference to a disease, disorder or symptoms thereof, refers to a reduction in the likelihood that an individual will develop a disease or disorder, e.g., a hemoglobinopathy. The likelihood of developing a disease or disorder is reduced, for example, when an individual having one or more risk factors for a disease or disorder either fails to develop the disorder or develops such disease or disorder at a later time or with less severity, statistically speaking, relative to a population having the same risk factors and not receiving treatment as described herein. The failure to develop symptoms of a disease, or the development of reduced (e.g., by at least 10% on a clinically accepted scale for that disease or disorder) or delayed (e.g., by days, weeks, months or years) symptoms is considered effective prevention.

In connection with contacting a cell with a DNA-targeting endonuclease to decrease BCL11A expression, the phrase “increasing fetal hemoglobin levels in a cell” indicates that fetal hemoglobin in a cell or population of cells is at least 5% higher in the cell or population of cells treated with the DNA-targeting endonuclease, than a comparable, control population, wherein no DNA-targeting endonuclease is present. It is preferred that the fetal hemoglobin expression in a DNA-targeting endonuclease treated cell is at least 10% higher, at least 20% higher, at least 30% higher, at least 40% higher, at least 50% higher, at least 60% higher, at least 70% higher, at least 80% higher, at least 90% higher, at least 1-fold higher, at least 2-fold higher, at least 5-fold higher, at least 10 fold higher, at least 100 fold higher, at least 1000-fold higher, or more than a comparable control treated population. The term “control treated population” is used herein to describe a population of cells that has been treated with identical media, viral induction, nucleic acid sequences, temperature, confluence, flask size, pH, etc., with the exception of the addition of the BCL11A inhibitor.

The term “mammal” is intended to encompass a singular “mammal” and plural “mammals,” and includes, but is not limited to humans; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and bears. In some preferred embodiments, a mammal is a human.

Accordingly, in one embodiment, the mammal has been diagnosed with a hemoglobinopathy. In a further embodiment, the hemoglobinopathy is a β-hemoglobinopathy. In one preferred embodiment, the hemoglobinopathy is a sickle cell disease. As used herein, “sickle cell disease” can be sickle cell anemia, sickle-hemoglobin C disease (HbSC), sickle beta-plus-thalassemia (HbS/β+), or sickle beta-zero-thalassemia (HbS/β0). In another preferred embodiment, the hemoglobinopathy is a β-thalassemia.

As used herein, the term “hemoglobinopathy” means any defect in the structure or function of any hemoglobin of an individual, and includes defects in the primary, secondary, tertiary or quaternary structure of hemoglobin caused by any mutation, such as deletion mutations or substitution mutations in the coding regions of the β-globin gene, or mutations in, or deletions of, the promoters or enhancers of such genes that cause a reduction in the amount of hemoglobin produced as compared to a normal or standard condition. The term further includes any decrease in the amount or effectiveness of hemoglobin, whether normal or abnormal, caused by external factors such as disease, chemotherapy, toxins, poisons, or the like.

In one embodiment, the term “effective amount”, as used herein, refers to the amount of a cell composition that is safe and sufficient to treat, lesson the likelihood of, or delay the development of a hemoglobinopathy. The amount can thus cure or result in amelioration of the symptoms of the hemoglobinopathy, slow the course of hemoglobinopathy disease progression, slow or inhibit a symptom of a hemoglobinopathy, slow or inhibit the establishment of secondary symptoms of a hemoglobinopathy or inhibit the development of a secondary symptom of a hemoglobinopathy. The effective amount for the treatment of the hemoglobinopathy depends on the type of hemoglobinopathy to be treated, the severity of the symptoms, the subject being treated, the age and general condition of the subject, the mode of administration and so forth. Thus, it is not possible or prudent to specify an exact “effective amount”. However, for any given case, an appropriate “effective amount” can be determined by one of ordinary skill in the art using only routine experimentation.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the technology.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined within the description of the various aspects and embodiments of the technology of the following.

### BRIEF DESCRIPTION OF DRAWINGS

FIGS. 1A-1C show identification of efficient BCL11A enhancer +55 edits for HbF induction in human CD34+ HSPCs. (FIG. 1A) Schematic representation of SpCas9 NGG PAM restricted chimeric single guide RNA (sgRNA) protospacer target sequences at BCL11A+55 enhancer. The half E-box/GATA motif instance (TGN₇₋₉WGATAR) which is the binding site for GATA1/TAL1 transcriptional activating complexes in erythroid cells is indicated by box. Targeting these sequences by sgRNAs #0086, #1448, #1449 and #1450 give especially high HbF induction. (FIG. 1B) Frequencies of indels by Sanger sequencing testing 16 sgRNAs targeting +55 enhancer in CD34+ HSPCs from three independent healthy donors. (FIG. 1C) HbF induction by 16 tested sgRNAs editing in in vitro differentiated erythroid progeny of edited CD34+ HSPCs from three independent healthy donors.

FIGS. 2A and 2B show potent HbF induction after combined editing of the +58 and +55 BCL11A erythroid enhancers. (FIG. 2A) HbF induction by HPLC analysis in erythroid cells in vitro differentiated from 3×NLS-SpCas9:sgRNA ribonucleoprotein (RNP) electroporated CD34+ HSPCs with sgRNAs indicated (the exceptions are “mock” which indicates negative control cells without genetic perturbation and “shmiR-BCL11A” which indicates lentiviral transduction with a microRNA embedded short hairpin RNA targeting BCL11A for post-transcriptional genetic silencing, per Esrick et al NEJM 2021, PMID: 33283990). #1617 and #1618 target the BCL11A +58 erythroid enhancer and #1449 and #1450 target the BCL11A +55 erythroid enhancer. The greatest HbF induction was produced by gene editing with RNPs combining 3×NLS-SpCas9 with pairs of sgRNAs targeting both the +58 and +55 BCL11A erythroid enhancers, such as sgRNAs #1617 and #1449, #1617 and #1450, #1618 and #1450, #1618 and #1449. ***P<0.0001, 1617+1449 compared to each of shMIR-BCL11A, HBG-115, 1617, 1618, 1449 and 1450 strategies. ****P<0.0001, 1617+1450, 1618+1450 and 1618+1449 compared to each of shMIR-BCL11A, HBG1-115, 1617, 1618, 1449 and 1450 strategies. (FIG. 2B) HbF level from individual erythroid single cell derived clonal liquid cultures derived from single CD34+ HSPCs from two SCD patients and five healthy donors electroporated with 3×NLS-SpCas9:sgRNA. Consistent with the bulk culture analyses, clonal analyses reveal that the greatest HbF induction was produced by gene editing with RNPs combining 3×NLS-SpCas9 with pairs of sgRNAs targeting both the +58 and +55 BCL11A erythroid enhancers, such as sgRNAs #1617 and #1449, #1617 and #1450, #1618 and #1450, #1618 and #1449. NS, nonsignificant, 1617+1449 compared to shMIR-BCL11A. 1617+1449 compared to each of HBG1-115, 1617, 1450 at least *P<0.05.1617+1450 compared to each of shMIR-BCL11A, HBG1-115, 1617 and 1450 at least *P<0.05. 1618+1450 and 1618+1449 compared to each of shMIR-BCL11A, HBG1-115, 1617 and 1450 at least ****P<0.0001.

FIGS. 3A-3D show genotyping analysis of erythroid cells derived from single CD34+ HSPC derived clones with combined editing at BCL11A +58 and +55 by 3×NLS-SpCas9 and two sgRNAs. These results show that precise 3.1 kb programmed deletions between the +58 and +55 target sequences (“deletion”), precise 3.1 kb programmed inversions between the +58 and +55 target sequences (“inversion”), and short insertions and deletions (“indel”) at each of the two target sites at +58 and +55 are each major allelic outcomes of combined +58 and +55 gene editing, with a minority of unedited alleles. (FIG. 3A) Allelic analysis of single clones after 1617+1449 editing. (FIG. 3B) Allelic analysis of single clones after 1617+1450 editing. (FIG. 3C) Allelic analysis of single clones after 1618+1449 editing. (FIG. 3D) Allelic analysis of single clones after 1618+1450 editing.

FIGS. 4A and 4B show the presence of TGN₇₋₉WGATAR half E-box/GATA motif following combined BCL11A +58 and 55 editing with sgRNAs #1617 and #1450. This scheme indicates that indels and precise 3.1 kb deletions disrupt both TGN₇₋₉WGATAR motifs at the +58 and +55 enhancers. Perfect precise 3.1 kb inversions between the BCL11A +58 and +55 deletions disrupt both TGN₇₋₉WGATAR motifs at the +58 and +55 enhancers, while imperfect inversion junctions with −1 bp deletion at +55 or +1 bp insertions at +58 can preserve motifs. (FIG. 4A) Schematic representation of TGN₇₋₉WGATAR motif disruption or preservation after 1617+1450 editing. (FIG. 4B) Junctional analysis of TGN₇₋₉WGATAR half E-box/GATA binding motifs in individual single cell derived erythroid colonies after editing of CD34+ HSPCs

FIGS. 5A and 5B show the presence of TGN₇₋₉WGATAR half E-box/GATA motif following combined BCL11A +58 and 55 editing with sgRNAs #1618 and #1450. (FIG. 5A) Schematic representation of TGN₇₋₉WGATAR motif disruption or preservation after 1618+1450 editing. (FIG. 5B) Junctional analysis of TGN₇₋₉WGATAR half E-box/GATA binding motifs. This scheme indicates that precise 3.1 kb deletions disrupt both TGN₇₋₉WGATAR motifs at the +58 and +55 enhancers. Indels may fully disrupt the TGN₇₋₉WGATAR motif, partially disrupt the motif to WGATAR-only motif, or retain the TGN₇₋₉WGATAR motif depending on the nature of the indel. Perfect precise 3.1 kb inversions between the BCL11A +58 and +55 deletions partially disrupt both TGN₇₋₉WGATAR motifs at the +58 and +55 enhancers converting them to WGATAR only motifs, while imperfect inversion junctions with −1 bp deletion at +55 or +1 bp insertions at +58 can reconstitute motifs. (FIG. 5A) Schematic representation of TGN₇₋₉WGATAR motif disruption or preservation after 1617+1450 editing. (FIG. 5B) Junctional analysis of TGN₇₋₉WGATAR half E-box/GATA binding motifs in individual single cell derived erythroid colonies after editing of CD34+ HSPCs.

FIGS. 6A-6D show the presence of TGN₇₋₉WGATAR half E-box/GATA motif is strongest predictor of residual enhancer function. (FIG. 6A) The association of HbF induction with genotype in clone after 1617+1450, 1618+1450 and 1617+1449 editing. (FIG. 6B) The correlation of HbF level with intact TGN₇₋₉WGATAR half E-box/GATA binding motifs in inversion clones of 1617+1449 editing. (FIGS. 6C and 6D). HbF level in clones based on number of TGN₇₋₉WGATAR half E-box/GATA binding motifs intact (maximum is 4) as well as partially disrupted WGATAR-only motifs at BCL11A +58 and +55 enhancers following 1617+1450 editing (FIG. 6C) and 1618+1450 editing (FIG. 6D). Ind2 refers to indels at both cleavage junctions.

FIGS. 7A-7G show comparisons of therapeutic strategies on HbF induction and engraftment in vivo. CD34+ HSPCs from 8 donors (6 healthy donors and 2 SCD donors as indicated) were treated as indicated with mock as negative control, shmiR-BCL11A transduced with lentiviral construct knocking down erythroid BCL111A expression, and otherwise gene edited with 3×NLS-SpCas9 and indicated sgRNAs, including HBG1-115 targeting the BCL11A binding site at the HBG1/2 promoters (Metais et al, Blood Adv 2019, PMID: 31698466), or indicated combination of BCL11A enhancer targeting sgRNAs #1617, #1618, #1449, #1450. CD34+ HSPCs are then infused to immunodeficient NBSGW mice. After 16 weeks, bone marrow is isolated and analyzed by flow cytometry evaluating total human (hCD45/CD45), HSPC (CD34/hCD45), erythroid (hCD235a/CD45−), B-cell (CD20/hCD45), granulocyte (CD33+SSC^{hi}/hCD45), and monocyte (CD33+SSC^{lo}/hCD45) repopulation. HbF measured by HPLC from MACS sorted hCD235a+ cells. The conclusion is that combined editing of BCL11A +58 and +55 enhancers gives the greatest HbF induction of any approach while preserving hematopoietic self-renewal and multilineage engraftment. (FIG. 7A) HbF induction in engrafted erythroid cells 16 weeks after transplant. ****P<0.0001 combined editing of BCL11A +58 and +55 enhancers compared to each of shMIR-BCL11A, HBG1-115, 1617, 1449, 1450 strategies. (FIG. 7B) Human chimerism in mouse BM 16 weeks after transplant. (FIG. 7C-7G) HSPCs, erythroid, B cells, granulocytes and monocytes engraftment in mouse BM 16 weeks after transplant.

FIGS. 8A and 8B show editing in engrafted cells as compared with input HSPCs. Engraftment experiment per FIGS. 7A-7G. (FIG. 8A) Frequency of indels by Sanger sequencing, deletion and inversion by digital drop PCR (ddPCR) assays in engrafted BM cells and input CD34+ HSPCs. Symbols indicate the donors of input and mice of engrafted. (FIG. 8B) Alleles repaired by NHEJ and MMEJ in engrafted BM cells and input CD34+ HSPCs, indel length from −8 to +6 bp was calculated as NHEJ, and from −9 to −20 bp as MMEJ. Symbols indicate the donors of input and mice of engrafted.

FIGS. 9A and 9B show human chimerism and editing after secondary transplantation. Bone marrow from primarily engrafted recipients after 16 weeks was used for secondary engraftment of NBSGW recipients from 3 healthy and two SCD donors. (FIG. 9A) Human chimerism in mouse BM 16 weeks after secondary transplantation. (FIG. 9B) Frequency of indels by Sanger sequencing, deletion and inversion by ddPCR assays in secondary engrafted BM cells. Symbols indicate the mice of secondary transplanted.

FIGS. 10A-10F show manufacturing process development. Gene editing of CD34+ HSPCs was performed under clinically relevant conditions with clinical grade RNP and electroporator (MaxCyte). Experiments in FIGS. 10A-10C with clinical scale (MaxCyte CL1.1) and experiments in FIGS. 10D-10F at small scale (MaxCyte OC-100). (FIG. 10A) Cell viability 24 hours post MaxCyte clinical-scale (CL1.1) 3×NLS-SpCas9:sgRNA RNP electroporation. (FIG. 10B) The frequencies of 3.1 kb deletion and 3.1 kb inversion by ddPCR, overall +58 editing by ddPCR. Unedited allele frequency defined as 1−overall editing frequency. The frequency of indels was defined as overall editing frequency −3.1 kb deletion frequency −3.1 kb inversion frequency. (FIG. 10C) HbF induction in in vitro differentiated erythroid cells after clinical-scale CD34+ cell RNP electroporation. (FIG. 10D) Cell viability 24 hours post small-scale CD34+ cell RNP electroporation. (FIG. 10E) The frequencies of 3.1 kb deletion and 3.1 kb inversion and indels after small-scale CD34+ cell RNP electroporation. (FIG. 10F) HbF induction in in vitro differentiated erythroid cells after small scale CD34+ cell RNP electroporation.

FIGS. 11A-11L show combined editing of BCL11A +58 and +55 enhancers is compatible with HSC self-renewal and multilineage differentiation in NBSGW xenotransplant. Experiments are collated from 12 human donors (8 healthy donors, 2 with SCD, 2 with beta-thalassemia). (FIG. 11A) Human chimerism 16 weeks after transplantation. (FIG. 11B-11F) B-cell lineage (FIG. 11B), granulocytes (FIG. 11C), monocytes (FIG. 11D), HSPCs (FIG. 11E), erythroid (FIG. 11F) engraftment in BM. (FIG. 11G) HbF levels in engrafted erythroid cells. (FIG. 11H) Overall +58 editing in engrafted BM cells as compared to input cells. (FIG. 11I) The frequencies of 3.1 kb deletion, 3.1 kb inversion and indels in engrafted BM cells as compared to input cells. Symbols indicate the donors of input and mice of engrafted. (FIG. 11J) Human chimerism 16 weeks after secondary transplant. (FIG. 11K) Multilineage engraftment after secondary transplant. (FIG. 11L) Overall +58 editing in secondary BM.

FIGS. 12A and 12B show off-target analysis of combined BCL11A +58 and +55 enhancer editing. Candidate off-target sites nominated by CRISPRme, GUIDE-seq, and ONE-seq were subject to pooled amplicon sequencing in at least 3 donors following 3×NLS-SpCas9:sgRNA RNP editing targeting the BCL11A +58 and +55 enhancers. The mean difference between indels in edited and control samples is shown for (FIG. 12A) 349 candidate off-target sites for +58 targeting (#1617) and (FIG. 12B) 239 candidate off-target sites for +55 targeting (#1450). The only off-target site identified at >0.1% indels was for 1617_OT_0040 which is an allele-specific off-target site specific for the minor allele rs114518452-C which generates a PAM sequence NGG for SpCas9.

FIGS. 13A-13F show combined editing of the +58 and +55 BCL11A erythroid enhancers in rhesus macaque autologous CD34+ HSPCs transplantation. These results show that efficient combined BCL11A +58 and +55 gene edits may be produced in a non-human primate autologous hematopoietic transplantation setting with robust HbF induction without any erythroid or other toxicity observed. HbF responses may be further enhanced during early repopulation post-transplantation (which is known to be associated with accelerated “stress” erythropoiesis kinetics) and by treatment with phlebotomy and hydroxyurea. (FIG. 13A) Schematic representation of G-CSF and plerixafor mobilization of rhesus CD34+ HSPCs, followed by 3×NLS-SpCas9:sgRNA RNP electroporation targeting the BCL11A +58 and +55 enhancers. After busulfan conditioning, autologous cryopreserved cells are thawed and infused. (FIG. 13B) Editing in sorted linages from engrafted bone marrow of two animals. (FIG. 13C) Allelic analysis from methylcelluolose colonies derived from bone marrow 51 weeks after autologous transplantation of ZL41. (FIG. 13D) Frequency of 3.1 kb deletion, 3.1 kb inversion, indels and unedited in engrafted granulocytes as compared to input cells by dPCR assays. (FIG. 13E) Editing of PB granulocytes and mononuclear cells, fraction of F cells and γ-globin induction, CBC analysis (hemoglobin and reticulocytes) for 3 animals (ZL41, 13U018, RA0013) after autologous transplantation. Two animals treated with hydroxyurea with phlebotomy (ZL41 and 13U018) and one treated with hydroxyurea without phlebotomy (RA0013) at indicated timepoints (FIG. 13F).

### DETAILED DESCRIPTION

### Hemoglobinopathies

The methods, vectors, RNPs and compositions described herein relate, in part, to the discovery that the combination targeting of at least two functional regions in BCL11A, e.g., location 60725424 to 60725688 (+55 functional region), location 60722238 to 60722466 (+58 functional region), and location 60718042 to 60718186 (+62 functional region) on the human chromosome 2 according to UCSC Genome Browser hg 19 human genome assembly, results in superior gene editing as compared to targeting a single functional region. The BCL11A protein acts as a stage specific regulator of fetal hemoglobin expression by repressing γ-globin induction, and thus is an ideal target for the treatment of hemoglobinopathies.

Fetal hemoglobin (HbF) is a tetramer of two adult a-globin polypeptides and two fetal β-like γ-globin polypeptides. During gestation, the duplicated γ-globin genes constitute the predominant genes transcribed from the β-globin locus. Following birth, γ-globin becomes progressively replaced by adult β-globin, a process referred to as the “fetal switch” (3). The molecular mechanisms underlying this switch have remained largely undefined and have been a subject of intense research. The developmental switch from production of predominantly fetal hemoglobin or HbF (α2γ2) to production of adult hemoglobin or HbA (α2β2) begins at about 28 to 34 weeks of gestation and continues shortly after birth at which point HbA becomes predominant. This switch results primarily from decreased transcription of the gamma-globin genes and increased transcription of beta-globin genes. On average, the blood of a normal adult contains only about 2% HbF, though residual HbF levels have a variance of over 20 fold in healthy adults (Atweh, Semin. Hematol. 38(4):367-73 (2001)).

Hemoglobinopathies encompass a number of anemias of genetic origin in which there is a decreased production and/or increased destruction (hemolysis) of red blood cells (RBCs). These disorders also include genetic defects that result in the production of abnormal hemoglobins with a concomitant impaired ability to maintain oxygen concentration. Some such disorders involve the failure to produce normal β-globin in sufficient amounts, while others involve the failure to produce normal β-globin entirely. These disorders specifically associated with the β-globin protein are referred to generally as β-hemoglobinopathies. For example, β-thalassemias result from a partial or complete defect in the expression of the β-globin gene, leading to deficient or absent HbA. Sickle cell anemia results from a point mutation in the β-globin structural gene, leading to the production of an abnormal (sickled) hemoglobin (HbS). HbS RBCs are more fragile than normal RBCs and undergo hemolysis more readily, leading eventually to anemia (Atweh, Semin. Hematol. 38(4):367-73 (2001)). Moreover, the presence of a BCL11A genetic variant, HBS1L-MYB variation, ameliorates the clinical severity in beta-thalassemia. This variant has been shown to be associated with HbF levels. It has been shown that there is an odds ratio of 5 for having a less severe form of beta-thalassemia with the high-HbF variant (Galanello S. et al., 2009, Blood, in press).

The search for treatment aimed at reduction of globin chain imbalance in patients with β-hemoglobinopathies has focused on the pharmacologic manipulation of fetal hemoglobin (α2γ2; HbF). The important therapeutic potential of such approaches is indicated by observations of the mild phenotype of individuals with co-inheritance of both homozygous β-thalassemia and hereditary persistence of fetal hemoglobin (HPFH), as well as by those patients with homozygous β-thalassemia who synthesize no adult hemoglobin, but in whom a reduced requirement for transfusions is observed in the presence of increased concentrations of fetal hemoglobin. Furthermore, it has been observed that certain populations of adult patients with β chain abnormalities have higher than normal levels of fetal hemoglobin (HbF), and have been observed to have a milder clinical course of disease than patients with normal adult levels of HbF. For example, a group of Saudi Arabian sickle-cell anemia patients who express 20-30% HbF have only mild clinical manifestations of the disease (Pembrey, et al., Br. J. Haematol. 40: 415-429 (1978)). It is now accepted that β-hemoglobinopathies, such as sickle cell anemia and the β-thalassemias, are ameliorated by increased HbF production. (Reviewed in Jane and Cunningham Br. J. Haematol. 102: 415-422 (1998) and Bunn, N. Engl. J. Med. 328: 129-131 (1993)).

As used herein, treating or reducing a risk of developing a hemoglobinopathy in a subject means to ameliorate at least one symptom of hemoglobinopathy. In one aspect, the invention features methods of treating, e.g., reducing severity or progression of, a hemoglobinopathy in a subject. In another aspect, the methods can also be used to reduce a risk of developing a hemoglobinopathy in a subject, delaying the onset of symptoms of a hemoglobinopathy in a subject, or increasing the longevity of a subject having a hemoglobinopathy. In one aspect, the methods can include selecting a subject on the basis that they have, or are at risk of developing, a hemoglobinopathy, but do not yet have a hemoglobinopathy, or a subject with an underlying hemoglobinopathy. Selection of a subject can include detecting symptoms of a hemoglobinopathy, a blood test, genetic testing, or clinical recordings. If the results of the test(s) indicate that the subject has a hemoglobinopathy, the methods also include administering the compositions described herein, thereby treating, or reducing the risk of developing, a hemoglobinopathy in the subject. For example, a subject who is diagnosis of SCD with genotype HbSS, HbS/P0 thalassemia, HbSD, or HbSO, and/or HbF <10% by electrophoresis.

As used herein, the term “hemoglobinopathy” refers to a condition involving the presence of an abnormal hemoglobin molecule in the blood. Examples of hemoglobinopathies include, but are not limited to, SCD and THAL. Also included are hemoglobinopathies in which a combination of abnormal hemoglobins is present in the blood (e.g., sickle cell/Hb-C disease). An exemplary example of such a disease includes, but is not limited to, SCD and THAL. SCD and THAL and their symptoms are well-known in the art and are described in further detail below. Subjects can be diagnosed as having a hemoglobinopathy by a health care provider, medical caregiver, physician, nurse, family member, or acquaintance, who recognizes, appreciates, acknowledges, determines, concludes, opines, or decides that the subject has a hemoglobinopathy.

The term “SCD” is defined herein to include any symptomatic anemic condition which results from sickling of red blood cells. Manifestations of SCD include: anemia; pain; and/or organ dysfunction, such as renal failure, retinopathy, acute-chest syndrome, ischemia, priapism, and stroke. As used herein the term “SCD” refers to a variety of clinical problems attendant upon SCD, especially in those subjects who are homozygotes for the sickle cell substitution in HbS. Among the constitutional manifestations referred to herein by use of the term of SCD are delay of growth and development, an increased tendency to develop serious infections, particularly due to pneumococcus, marked impairment of splenic function, preventing effective clearance of circulating bacteria, with recurrent infarcts and eventual destruction of splenic tissue. Also included in the term “SCD” are acute episodes of musculoskeletal pain, which affect primarily the lumbar spine, abdomen, and femoral shaft, and which are similar in mechanism and in severity. In adults, such attacks commonly manifest as mild or moderate bouts of short duration every few weeks or months interspersed with agonizing attacks lasting 5 to 7 days that strike on average about once a year. Among events known to trigger such crises are acidosis, hypoxia, and dehydration, all of which potentiate intracellular polymerization of HbS (J. H. Jandl, Blood: Textbook of Hematology, 2nd Ed., Little, Brown and Company, Boston, 1996, pages 544-545).

As used herein, “THAL” refers to a hereditary disorder characterized by defective production of hemoglobin. In one embodiment, the term encompasses hereditary anemias that occur due to mutations affecting the synthesis of hemoglobins. In other embodiments, the term includes any symptomatic anemia resulting from thalassemic conditions such as severe or β-thalassemia, thalassemia major, thalassemia intermedia, a-thalassemias such as hemoglobin H disease. β-thalassemias are caused by a mutation in the β-globin chain, and can occur in a major or minor form. In the major form of β-thalassemia, children are normal at birth, but develop anemia during the first year of life. The mild form of β-thalassemia produces small red blood cells. Alpha-thalassemias are caused by deletion of a gene or genes from the globin chain.

By the phrase “risk of developing disease” is meant the relative probability that a subject will develop a hemoglobinopathy in the future as compared to a control subject or population (e.g., a healthy subject or population). For example, an individual carrying the genetic mutation associated with SCD, an A to T mutation of the β-globin gene, and whether the individual in heterozygous or homozygous for that mutation increases that individual's risk.

### Vectors

Several aspects of the invention relate vectors comprising at least two guide RNAs (gRNAs) that hybridize to at least two target sequences on a DNA molecule, wherein each of the at least two gRNAs hybridizes at least two genomic DNA locations selected from human chromosome 2 at location 60725424 to 60725688 according to GRCh37/hg19 human genome reference build (+55 functional region); human chromosome 2 at location 60722238 to 60722466 GRCh37/hg19 human genome reference build (+58 functional region); or human chromosome 2 at location 60718042 to 60718186 according to GRCh37/hg19 human genome reference build (+62 functional region).

In one embodiment, the vector further comprises a DNA endonuclease.

Vectors may be introduced and propagated in a prokaryote. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell (e.g., a quiescent cell) or host organism. Expression of polypeptides in prokaryotes is most often carried out in Escherichia coli with vectors containing constitutive or inducible promoters directing the expression of either fusion or non-fusion proteins. Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway. N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET I1d (Studier et al., GENE EXPRESSION TECHNOLOGY: METHODS IN ENZYMOLOGY 185, Academic Press, San Diego, Calif. (1990) 60-89).

In some embodiments, a vector is a yeast expression vector. Examples of vectors for expression in yeast Saccharomyces cerivisae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

In some embodiments, a vector drives polypeptide (i.e., protein) expression in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of a nucleic acid preferentially in a particular cell type (e.g., tissue-specific regulatory elements are used to express the nucleic acid in, for example, a hematopoietic cell). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The eukaryotic cells may be those of or derived from a particular organism, such as a mammal, including but not limited to human, mouse, rat, rabbit, dog, or non-human primate.

In one embodiment, the vector described herein are introduced to a cell via a non-viral method. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024.

Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

In some embodiments, a cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panel, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4. COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-I cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L235010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

Yet another aspect described herein is a composition comprising any vector described herein. In one embodiment, the composition further comprises a pharmaceutical acceptable carrier.

Yet another aspect described herein is a pharmaceutical composition comprising any vector described herein.

### Ribonucleoprotein (RNP) Complex

One aspect ribonucleoprotein (RNP) complex comprising a DNA endonuclease enzyme and at least two guide RNAs (gRNAs) that targets and hybridizes to a target sequence on a DNA molecule, wherein each of the at least two gRNAs hybridizes at least two genomic DNA locations selected from human chromosome 2 at location 60725424 to 60725688 according to GRCh37/hg19 human genome reference build (+55 functional region); human chromosome 2 at location 60722238 to 60722466 GRCh37/hg19 human genome reference build (+58 functional region); or human chromosome 2 at location 60718042 to 60718186 according to GRCh37/hg19 human genome reference build (+62 functional region).

In one embodiment, the RNP is sued for altering expression of at least one gene product, e.g., in any of the cells described herein.

Another aspect described herein is a vector comprising any of the RNPs described herein.

RNPs described herein can be introduced into a cell using any standard method known in the art, for example, electroporation, lipofection. RNPs can also be introduced into a cell using any viral or non-viral method as described herein above.

Yet another aspect described herein is a composition comprising any vector comprising an RNP as described herein. In one embodiment, the composition further comprises a pharmaceutical acceptable carrier.

Yet another aspect described herein is a pharmaceutical composition comprising any vector comprising an RNP as described herein.

Guide RNAs (gRNAs)

In one embodiment, the gRNAs of the invention hybridizes at at least two genomic DNA locations selected from human chromosome 2 at location 60725424 to 60725688 according to GRCh37/hg19 human genome reference build (+55 functional region); human chromosome 2 at location 60722238 to 60722466 GRCh37/hg19 human genome reference build (+58 functional region); or human chromosome 2 at location 60718042 to 60718186 according to GRCh37/hg19 human genome reference build (+62 functional region).

As used herein, “hybridizes” or “hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

The sequence of the guide RNA (e.g., the sequence homologous to the target gene of interest) can be determined for the intended use. For example, to target the BCL11A gene, one would choose a guide RNA that targets and hybridize to BCL11A in a manner that effectively results in the desired alteration of the gene's expression. In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site of the form MMMMMMMMNNNNNNNNNNNNXGG where NNNNNNNNNNNNXGG (N is A, G, T, or C; and X can be anything) has a single occurrence in the genome. A unique target sequence in a genome may include an S. pyogenes Cas9 target site of the form MMMMMMMMMNNNNNNNNNNNXGG where NNNNNNNNNNNXGG (N is A, G, T, or C; and X can be anything) has a single occurrence in the genome. Alternatively, the first 8 positions in the above mentioned unique sequences can be NNNNNNNN, for example, NNNNNNNNNNNNNNNNNNNNXGG.

In one embodiment, at vector or RNP of the invention comprise at least two gRNA, e.g., comprises 2, 3, 4, 5 or more gRNAs. In one embodiment, the at least two gRNAs target the same genomic DNA location, e.g., the at least two gRNAs each target the +55 functional region. In one embodiment, the at least two gRNAs each target different genomic DNA locations, e.g., at least one gRNA targets the +55 functional region, and at least one gRNA targets the +58 functional region.

In one embodiment, at vector or RNP of the invention comprises at least one gRNA that hybridizes to the +55 functional region and at least one gRNA that hybridizes to the +58 functional region.

In one embodiment, at vector or RNP of the invention comprises at least one gRNA that hybridizes to the +55 functional region and at least one gRNA that hybridizes to the +62 functional region.

In one embodiment, at vector or RNP of the invention comprises at least one gRNA that hybridizes to the +58 functional region and at least one gRNA that hybridizes to the +62 functional region.

In one embodiment, the RNP has one gRNA that hybridizes to the +55 functional region, one gRNA that hybridizes to the +58 functional region, one gRNA that hybridizes to the +62 functional region.

In one embodiment, the gRNA comprises, consists of, or consists essentially of a sequence selected from Table 1.

TABLE 1
Exemplary gRNAs

| | | SEQ ID |
|---|---|---|
| gRNA | Sequence | NO: |
| #1617 | CUAACAGUUGCUUUUAUCAC | 1 |
| #1618 | UUGCUUUUAUCACAGGCUCC | 2 |
| #1449 | GUGCUUGGUCGGCACUGAUA | 3 |
| #1450 | UGCUUGGUCGGCACUGAUAG | 4 |

In one embodiment, the vector or RNP of the invention comprises at least a gRNA having a sequence of SEQ ID NO: 1 and a gRNA having a sequence of SEQ ID NO: 2.

In one embodiment, the vector or RNP of the invention comprises at least a gRNA having a sequence of SEQ ID NO: 1 and a gRNA having a sequence of SEQ ID NO: 3.

In one embodiment, the vector or RNP of the invention comprises at least a gRNA having a sequence of SEQ ID NO: 1 and a gRNA having a sequence of SEQ ID NO: 4.

In one embodiment, the vector or RNP of the invention comprises at least a gRNA having a sequence of SEQ ID NO: 2 and a gRNA having a sequence of SEQ ID NO: 3.

In one embodiment, the vector or RNP of the invention comprises at least a gRNA having a sequence of SEQ ID NO: 2 and a gRNA having a sequence of SEQ ID NO: 4.

In one embodiment, the vector or RNP of the invention comprises at least a gRNA having a sequence of SEQ ID NO: 3 and a gRNA having a sequence of SEQ ID NO: 4.

SEQ ID NOs 36-170 (Table 3) or 171-199 (Table 4) described a target sequence for a gRNA, i.e., a sequence which the gRNA hybridizes to. In one embodiment, the gRNA has a sequence that is complementary to a sequence selected from a SEQ ID NOs 36-170 (Table 3) or 171-199 (Table 4).

### DNA Endonucleases

Genome editing cannot be performed using traditional restriction endonucleases since most restriction enzymes recognize a few base pairs on the DNA as their target and the probability is very high that the recognized base pair combination will be found in many locations across the genome resulting in multiple cuts (i.e., not limited to a desired location). To overcome this challenge and create site-specific double-stranded breaks, several distinct classes of nucleases have been discovered and bioengineered to date. These are the meganucleases, Zinc finger nucleases (ZFNs), Cas9/CRISPR system, and transcription-activator like effector nucleases (TALENs).

In one embodiment, the DNA endonuclease is selected from the group consisting of a Zinc Finger Nuclease (ZFN), a Transcription Activator-Like Effector Nuclease (TALENs), and a CRISPR-associated protein (Cas) nuclease.

Meganucleases are commonly grouped into four families: the LAGLIDADG family (“LAGLIDADG” disclosed as SEQ ID NO: 15), the GIY-YIG family, the His-Cys box family and the HNH family. These families are characterized by structural motifs, which affect catalytic activity and recognition sequence. For instance, members of the LAGLIDADG family (“LAGLIDADG” disclosed as SEQ ID NO: 15) are characterized by having either one or two copies of the conserved LAGLIDADG motif (see Chevalier et al. (2001), Nucleic Acids Res. 29(18): 3757-3774). The LAGLIDADG meganucleases (“LAGLIDADG” disclosed as SEQ ID NO: 15) with a single copy of the LAGLIDADG motif (SEQ ID NO: 15) form homodimers, whereas members with two copies of the LAGLIDADG (SEQ ID NO: 15) are found as monomers. Similarly, the GIY-YIG family members have a GIY-YIG module, which is 70-100 residues long and includes four or five conserved sequence motifs with four invariant residues, two of which are required for activity (see Van Roey et al. (2002), Nature Struct. Biol. 9: 806-811). The His-Cys box meganucleases are characterized by a highly conserved series of histidines and cysteines over a region encompassing several hundred amino acid residues (see Chevalier et al. (2001), Nucleic Acids Res. 29(18): 3757-3774). In the case of the NHN family, the members are defined by motifs containing two pairs of conserved histidines surrounded by asparagine residues (see Chevalier et al. (2001), Nucleic Acids Res. 29(18): 3757-3774). The four families of meganucleases are widely separated from one another with respect to conserved structural elements and, consequently, DNA recognition sequence specificity and catalytic activity.

Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision BioSciences' Directed Nuclease Editor™ genome editing technology.

ZFNs and TALENs restriction endonuclease technology utilizes a non-specific DNA cutting enzyme which is linked to a specific DNA sequence recognizing peptide(s) such as zinc fingers and transcription activator-like effectors (TALEs). Typically, an endonuclease whose DNA recognition site and cleaving site are separate from each other is selected and the its cleaving portion is separated and then linked to a sequence recognizing peptide, thereby yielding an endonuclease with very high specificity for a desired sequence. An exemplary restriction enzyme with such properties is FokI. Additionally, FokI has the advantage of requiring dimerization to have nuclease activity and this means the specificity increases dramatically as each nuclease partner recognizes a unique DNA sequence. To enhance this effect, FokI nucleases have been engineered that can only function as heterodimers and have increased catalytic activity. The heterodimer functioning nucleases avoid the possibility of unwanted homodimer activity and thus increase specificity of the double-stranded break.

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically happen in repeats that are 3 bp apart and are found in diverse combinations in a variety of nucleic acid interacting proteins such as transcription factors. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs for use with the methods and compositions described herein can be obtained commercially from e.g., Sangamo Biosciences™ (Richmond, CA).

Alternatively, genome editing can be performed using recombinant adeno-associated virus (rAAV) based genome engineering, which is a genome-editing platform centered around the use of rAAV vectors that enables insertion, deletion or substitution of DNA sequences into the genomes of live mammalian cells. The rAAV genome is a single-stranded deoxyribonucleic acid (ssDNA) molecule, either positive- or negative-sensed, which is about 4.7 kilobase long. These single-stranded DNA viral vectors have high transduction rates and have a unique property of stimulating endogenous homologous recombination in the absence of causing double strand DNA breaks in the genome. One of skill in the art can design a rAAV vector to target a desired genomic locus and perform both gross and/or subtle endogenous gene alterations in a cell, such as a deletion. rAAV genome editing has the advantage in that it targets a single allele and does not result in any off-target genomic alterations. rAAV genome editing technology is commercially available, for example, the rAAV GENESIS™ system from Horizon™ (Cambridge, UK).

The invention described herein improves on the widely-used CRISPR system for the use of gene-editing in the cell. By introducing at least two gRNAs, the inventors have found that they could unexpectedly, and dramatically increase the HbF expression in erythroid cells derived from edited hematopoietic stem and progenitor cells.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast. A sequence or template that may be used for recombination into the targeted locus comprising the target sequences is referred to as an “editing template” or “editing polynucleotide” or “editing sequence”. In aspects of the invention, an exogenous template polynucleotide may be referred to as an editing template. In an aspect of the invention the recombination is homologous recombination.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. As with the target sequence, it is believed that complete complementarity is not needed, provided there is sufficient to be functional. In some embodiments, the tracr sequence has at least 50%, 60%, 70%, 80%, 90%, 95% or 99% of sequence complementarity along the length of the tracr mate sequence when optimally aligned. In some embodiments, one or more vectors driving expression of one or more elements of a CRISPR system are introduced into a cell such that expression of the elements of the CRISPR system direct formation of a CRISPR complex at one or more target sites. For example, an NLS-Cas fusion enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence, and tracr sequence are operably linked to and expressed from the same promoter.

In one embodiment, the CRISPR enzyme is a type II CRISPR system enzyme.

In one embodiment, the CRISPR enzyme is a Cas protein. Non-limiting examples of Cas proteins include Cpf1, C2c1, C2c3, Cas12a, Cas12b, Cas12c, Cas12d, Cas12e, Cas13a, Cas13b, and Cas13c. Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c homologs thereof, or modified versions thereof. These enzymes are known; for example, the amino acid sequence of S. pyogenes Cas9 protein may be found in the SwissProt database under accession number Q99ZW2. In some embodiments, the CRISPR enzyme has DNA cleavage activity, such as Cas9. In some embodiments the CRISPR enzyme is Cas9, and may be Cas9 from S. pyogenes or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence.

### NLS-CRISPR Enzyme Fusion

Various aspects herein provide a CRISPR enzyme comprising at least one nuclear localization signal sequences (NLSs). In some embodiments, the CRISPR enzyme comprises at least one NLSs at or near the amino-terminus, at least one NLSs at or near the carboxy-terminus, or a combination of these (e.g. one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. Typically, an NLS consists of one or more short sequences of positively charged lysines or arginines exposed on the protein surface, but other types of NLS are known. Non-limiting examples of NLSs include an NLS sequence derived from: the NLS of the SV40 virus large T-antigen, having the amino acid sequence PKKKRKV (SEQ ID NO: 16); the NLS from nucleoplasmin (e.g. the nucleoplasmin bipartite NLS with the sequence KRPAATKKAGQAKKKK (SEQ ID NO: 17)); the c-myc NLS having the amino acid sequence PAAKRVKLD (SEQ ID NO: 18) or RQRRNELKRSP (SEQ ID NO: 19); the hRNPA1 M9 NLS having the sequence NQSSNFGPMKGGNFGGRSSGPYGGGGQYFAKPRNQGGY (SEQ ID NO: 20); the sequence RMRIZFKNKGKDTAELRRRRVEVSVELRKAKKDEQILKRRNV (SEQ ID NO: 21) of the IBB domain from importin-alpha; the sequences VSRKRPRP (SEQ ID NO: 22) and PPKKARED (SEQ ID NO: 23) of the myoma T protein; the sequence PQPKKKPL (SEQ ID NO: 24) of human p53; the sequence SALIKKKKKMAP (SEQ ID NO: 25) of mouse c-abl IV; the sequences DRLRR (SEQ ID NO: 26) and PKQKKRK (SEQ ID NO: 27) of the influenza virus NS1; the sequence RKLKKKIKKL (SEQ ID NO: 28) of the Hepatitis virus delta antigen; the sequence REKKKFLKRR (SEQ ID NO: 29) of the mouse Mx1 protein; the sequence KRKGDEVDGVDEVAKKKSKK (SEQ ID NO: 30) of the human poly(ADP-ribose) polymerase; the sequence RKCLQAGMNLEARKTKK (SEQ ID NO: 31) of the steroid hormone receptors (human) glucocorticoid; the sequence GKRKLITSEEERSPAKRGRKS (SEQ ID NO: 32) of 53BP1; the sequence KRKRRP (SEQ ID NO. 33) of BRCA1; the sequence KRKGSPCDTLASSTEKRRRE (SEQ ID NO. 34) of SRC-1; and the sequence KRNFRSALNRKE (SEQ ID NO: 35) of IRF3.

In general, the one or more NLSs are of sufficient strength to drive accumulation of the CRISPR enzyme in a detectable amount in the nucleus of a eukaryotic cell. In general, strength of nuclear localization activity may derive from the number of NLSs in the CRISPR enzyme, the particular NLS(s) used, or a combination of these factors. Detection of accumulation in the nucleus may be performed by any suitable technique. For example, a detectable marker may be fused to the CRISPR enzyme, such that location within a cell may be visualized, such as in combination with a means for detecting the location of the nucleus (e.g. a stain specific for the nucleus such as DAPI). Examples of detectable markers include fluorescent proteins (such as Green fluorescent proteins, or GFP; RFP; CFP), and epitope tags (HA tag, flag tag, SNAP tag). Cell nuclei may also be isolated from cells using standard methods (e.g., cell lysis and centrifugation spins to separate the nuclei and cytosol), the contents of which may then be analyzed by any suitable process for detecting protein, such as immunohistochemistry, Western blot, or enzyme activity assay. Accumulation in the nucleus may also be determined indirectly, such as by an assay for the effect of CRISPR complex formation (e.g. assay for DNA cleavage or mutation at the target sequence, or assay for altered gene expression activity affected by CRISPR complex formation and/or CRISPR enzyme activity), as compared to a control no exposed to the CRISPR enzyme or complex, or exposed to a CRISPR enzyme lacking the one or more NLSs.

In one embodiment, linkers are inserted in between at least one NLS sequence and the CRISPR enzyme sequence, and/or in between two NLS sequences. In one embodiment, at least 1, 2, 3, 4, 5, 6, 7, 8, 9, 10 or more linkers are included in the vector or RNPS described herein. When more than one linker is used, the more than one linkers can be identical, or the more than one linkers can be different. Table 2 below presents nucleotide and protein sequences for exemplary linkers. Various aspects described herein provides a composition comprising of, consisting of, or consisting essentially of any of the vectors or RNP complexes described herein.

TABLE 2
nucleotide and protein sequences for exemplary linkers

| Protein linker | Corresponding nucleic |
|---|---|
| sequences | acid linker sequences |
| Gly-Gly-Ser-Gly | Ggcggtagcggc |
| (SEQ ID NO: 5) | (SEQ ID NO: 10) |
| (Gly-Gly-Ser-Gly) × 3 | ggcggtagcggcggaggcagcggtggcggcagcggc |
| (SEQ ID NO: 6) | (SEQ ID NO: 11) |
| (Gly-Gly-Ser-Gly) × 5 | ggcggtagcggcggcggtagcggcggaggcagcggt |
| (SEQ ID NO: 7) | ggcggcagcggcggcggtagcggc (SEQ ID NO: |
| | 12) |
| TGGGPGGGAAAGSGS | accggtggtggtcccgggggtggtgcggccgcaggc |
| (SEQ ID NO: 8) | agcggaagc (SEQ ID NO: 13) |
| SGGSSGGSSGSETPGTSESATPE | Tctggaggatctagcggaggatcctctggaagcgag |
| SSGGSSGGS (SEQ ID NO: | acaccaggcacaagcgagtccgccacaccagagagc |
| 9) | tccggcggctcctccggaggatcc (SEQ ID NO: |
| | 14) |

### Compositions

Additional aspects described herein provides a composition comprising of, consisting of, or consisting essentially of any of the cells (e.g., genetically engineered cells) described herein.

In one embodiment, the compositions described herein further comprise a pharmaceutically acceptable carrier. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used with the methods described herein that will be effective in the treatment of a particular disorder or condition will depend on the nature of the disorder or condition, and can be determined by standard clinical techniques.

Also provides are pharmaceutical compositions comprising any of the vectors, RNPs or cells described herein.

### Methods

Aspects described herein are directed to methods of altering the expression of a gene product.

One aspect described herein is directed to a method for producing a progenitor cell having decreased BCL11A mRNA or protein expression, the method comprising contacting an isolated progenitor cell with any vector, RNP, or composition thereof described herein.

In one embodiment, BCL11A mRNA or protein expression is decreased by at least 10%. For example, BCL11A mRNA or protein expression is decreased by at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more than a control treated population of comparable size and culture conditions. The term “control treated population” is used herein to describe a population of cells that has been treated with identical media, viral induction, nucleic acid sequences, temperature, confluence, flask size, pH, etc., with the exception of any vector, RNP or composition described herein. A “control treated population” can also be one that is treated with a vector or RNP that comprises only one gRNA.

Another aspect described herein is directed to a method for producing an isolated genetic engineered human cell having at least one genetic modification comprising contacting an isolated cell with an effective amount of any vector, RNP, or composition thereof described herein, causing at least one genetic modification therein. Exemplary genetic modifications include an at least one base pair substitution, deletion or addition, e.g., that can lead to a disruption at the genomic level resulting in a decrease in BCL11A expression or activity in a cell.

In one embodiment, the at least one genetic modification is a base pair deletion, insertion, or inversion.

In one embodiment, the isolated progenitor cell or isolated cell is a hematopoietic progenitor cell.

In one embodiment, the hematopoietic progenitor is a cell of the erythroid lineage.

In one embodiment, the isolated progenitor cell or isolated cell is an induced pluripotent stem cell.

In one embodiment, the cell is a quiescent cell. As used herein, “quiescent cell” refers to a cell in a reversible state in which it does not divide but retains the ability to re-enter cell proliferation. Exemplary quiescent cells include, but are not limited to, a hematopoietic stem cell, a muscle stem cell, a neural stem cell, an intestinal stem cell, a skin stem cell or epidermal stem cell, a mesenchymal stem cell, a resting T cell, a memory T cell, a neuron, a neuronal stem cell, a myotube or skeletal myoblast or satellite cell, and a hepatocyte.

Another method for altering the expression of a gene product provided herein comprises introducing into a cell any of the vectors, RNP or compositions described herein.

It is specifically contemplated herein that any gene in the cell's genome can be altered using methods herein.

Another aspect described herein provides a method of increasing fetal hemoglobin levels in a cell, the method comprising the steps of: contacting an isolated cell with an effective amount of any vector, RNP or composition described herein, whereby fetal hemoglobin expression is increased in said cell, or its progeny, relative to said cell prior to said contacting.

Another aspect described herein provides a method for increasing fetal hemoglobin levels in a mammal in need thereof, the method comprising the steps of contacting an isolated hematopoietic progenitor cell in said mammal with an effective amount of any vector, RNP or composition described herein, causing at least one genetic modification therein, whereby fetal hemoglobin expression is increased in said mammal, relative to expression prior to said contacting.

Yet another aspect described herein provides a method for increasing fetal hemoglobin levels in a mammal in need thereof, the method comprising transplanting any isolated genetic engineered human cell produced using methods described herein into the mammal.

As used herein the term “increasing the fetal hemoglobin levels” in a cell indicates that fetal hemoglobin is at least 5% higher in populations treated with any vector, RNP or composition herein, than in a comparable, control population, wherein no any vector, RNP or composition is present. In one embodiment, the fetal hemoglobin expression in a population treated with any vector, RNP or composition described herein is at least at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more higher, or at least 1-fold higher, at least 2-fold higher, at least 5-fold higher, at least 10 fold higher, at least 100 fold higher, at least 1000-fold higher, than a control treated population of comparable size and culture conditions. The term “control treated population” is used herein to describe a population of cells that has been treated with identical media, viral induction, nucleic acid sequences, temperature, confluence, flask size, pH, etc., with the exception of any vector, RNP or composition described herein. A “control treated population” can also be one that is treated with a vector or RNP that comprises only one gRNA. A skilled artisan can use any method known in the art to measure an increase in fetal hemoglobin expression, e. g. Western Blot analysis of fetal γ-globin protein and quantifying mRNA of fetal γ-globin.

In one embodiment, contacting is in vivo. In one embodiment, contacting is in vitro. In one embodiment, contacting is ex vivo.

### Engineered Cells

In one embodiment vector, RNP complex, or a composition thereof described herein can be used to engineer a cell that has an altered gene expression as compared to a wild-type cell. In another embodiment, the methods described herein can be used to engineer a cell that has an altered gene expression as compared to a wild-type cell. For example, a quiescent cell can be engineered to have increased level of fetal hemoglobin using methods described herein. In one embodiment, the engineered cell is a quiescent cell, for example, a hematopoietic stem cell. In one embodiment, the engineered cell is a quiescent cell that can be administered to a subject in need thereof. In one embodiment, the engineered cell can be an isolated cell, or can be comprised in an isolated population.

The term “isolated cell” as used herein refers to a cell that has been removed from an organism in which it was originally found, or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched. In some embodiments, the isolated population is an isolated population of engineered human hematopoietic progenitor cells, e.g., a substantially pure population of engineered human hematopoietic progenitor cells as compared to a heterogeneous population of cells comprising engineered human hematopoietic progenitor cells and cells from which the human hematopoietic progenitor cells were derived.

Isolated populations of cells useful as a therapeutic are often desired to be substantially pure. The term “substantially pure,” with respect to a particular cell population, refers to a population of cells that is at least about 75%, preferably at least about 85%, more preferably at least about 90%, and most preferably at least about 95% pure, with respect to the cells making up a total cell population. That is, the terms “substantially pure” or “essentially purified,” with regard to a population of, for example, engineered hematopoietic progenitor cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not engineered hematopoietic progenitor cells as defined by the terms herein.

In one embodiment, the engineered cell can be comprised in a composition. In another embodiment, the engineered cell can be comprised in a pharmaceutical composition. A composition of cell described herein can further comprise a pharmaceutically acceptable carrier. It is desired that any pharmaceutically acceptable carrier used is beneficial in promoting the health and/or growth of the cells and does not result in an adverse effect or negatively impact the cells comprised in the composition. For example, a carrier that results in cell death or alters the physiological properties (e.g., size, shape, pH, etc.) would not be desired.

The disclosure described herein, in a preferred embodiment, does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

The disclosure described herein, in a preferred embodiment, does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Various aspects herein provide a composition of engineered cells produced using any method described herein.

One aspect provides a composition of any progenitor engineered human cells obtained by any method described herein.

One aspect provides a composition of any isolated genetic engineered human cells obtained by any method described herein.

### Hematopoietic Progenitor Cells

In one embodiment, the hematopoietic progenitor cell is contacted ex vivo or in vitro. In a specific embodiment, the cell being contacted is a cell of the erythroid lineage. In one embodiment, the cell composition comprises cells having decreased BCL11A expression.

“Hematopoietic progenitor cell” as the term is used herein, refers to cells of a stem cell lineage that give rise to all the blood cell types including the myeloid (monocytes and macrophages, neutrophils, basophils, eosinophils, erythrocytes, megakaryocytes/platelets, dendritic cells), and the lymphoid lineages (T-cells, B-cells, NK-cells). A “cell of the erythroid lineage” indicates that the cell being contacted is a cell that undergoes erythropoiesis such that upon final differentiation it forms an erythrocyte or red blood cell (RBC). Such cells belong to one of three lineages, erythroid, lymphoid, and myeloid, originating from bone marrow hematopoietic progenitor cells. Upon exposure to specific growth factors and other components of the hematopoietic microenvironment, hematopoietic progenitor cells can mature through a series of intermediate differentiation cellular types, all intermediates of the erythroid lineage, into RBCs. Thus, cells of the “erythroid lineage”, as the term is used herein, comprise hematopoietic progenitor cells, rubriblasts, prorubricytes, erythroblasts, metarubricytes, reticulocytes, and erythrocytes.

In some embodiment, the hematopoietic progenitor cell has at least one of the cell surface marker characteristic of hematopoietic progenitor cells: CD34+, CD59+, Thy1/CD90+, CD38lo/−, and C-kit/CD117+. Preferably, the hematopoietic progenitor cells have several of these markers.

In some embodiments, the hematopoietic progenitor cells of the erythroid lineage have the cell surface marker characteristic of the erythroid lineage: CD71 and Ter119.

Stem cells, such as hematopoietic progenitor cells, are capable of proliferation and giving rise to more progenitor cells having the ability to generate a large number of mother cells that can in turn give rise to differentiated or differentiable daughter cells. The daughter cells themselves can be induced to proliferate and produce progeny that subsequently differentiate into one or more mature cell types, while also retaining one or more cells with parental developmental potential. The term “stem cell” refers then, to a cell with the capacity or potential, under particular circumstances, to differentiate to a more specialized or differentiated phenotype, and which retains the capacity, under certain circumstances, to proliferate without substantially differentiating. In one embodiment, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Generally, “progenitor cells” have a cellular phenotype that is more primitive (i.e., is at an earlier step along a developmental pathway or progression than is a fully differentiated cell). Often, progenitor cells also have significant or very high proliferative potential. Progenitor cells can give rise to multiple distinct differentiated cell types or to a single differentiated cell type, depending on the developmental pathway and on the environment in which the cells develop and differentiate.

In the context of cell ontogeny, the adjective “differentiated”, or “differentiating” is a relative term. A “differentiated cell” is a cell that has progressed further down the developmental pathway than the cell it is being compared with. Thus, stem cells can differentiate to lineage-restricted precursor cells (such as a hematopoietic progenitor cell), which in turn can differentiate into other types of precursor cells further down the pathway (such as an erythrocyte precursor), and then to an end-stage differentiated cell, such as an erythrocyte, which plays a characteristic role in a certain tissue type, and may or may not retain the capacity to proliferate further.

### Induced Pluripotent Stem Cells

In some embodiments, the genetic engineered human cells described herein are derived from isolated pluripotent stem cells. An advantage of using iPSCs is that the cells can be derived from the same subject to which the progenitor cells are to be administered. That is, a somatic cell can be obtained from a subject, reprogrammed to an induced pluripotent stem cell, and then re-differentiated into a hematopoietic progenitor cell to be administered to the subject (e.g., autologous cells). Since the progenitors are essentially derived from an autologous source, the risk of engraftment rejection or allergic responses is reduced compared to the use of cells from another subject or group of subjects. In some embodiments, the hematopoietic progenitors are derived from non-autologous sources. In addition, the use of iPSCs negates the need for cells obtained from an embryonic source. Thus, in one embodiment, the stem cells used in the disclosed methods are not embryonic stem cells.

Although differentiation is generally irreversible under physiological contexts, several methods have been recently developed to reprogram somatic cells to induced pluripotent stem cells. Exemplary methods are known to those of skill in the art and are described briefly herein below.

As used herein, the term “reprogramming” refers to a process that alters or reverses the differentiation state of a differentiated cell (e.g., a somatic cell). Stated another way, reprogramming refers to a process of driving the differentiation of a cell backwards to a more undifferentiated or more primitive type of cell. It should be noted that placing many primary cells in culture can lead to some loss of fully differentiated characteristics. Thus, simply culturing such cells included in the term differentiated cells does not render these cells non-differentiated cells (e.g., undifferentiated cells) or pluripotent cells. The transition of a differentiated cell to pluripotency requires a reprogramming stimulus beyond the stimuli that lead to partial loss of differentiated character in culture. Reprogrammed cells also have the characteristic of the capacity of extended passaging without loss of growth potential, relative to primary cell parents, which generally have capacity for only a limited number of divisions in culture.

The cell to be reprogrammed can be either partially or terminally differentiated prior to reprogramming. In some embodiments, reprogramming encompasses complete reversion of the differentiation state of a differentiated cell (e.g., a somatic cell) to a pluripotent state or a multipotent state. In some embodiments, reprogramming encompasses complete or partial reversion of the differentiation state of a differentiated cell (e.g., a somatic cell) to an undifferentiated cell (e.g., an embryonic-like cell). Reprogramming can result in expression of particular genes by the cells, the expression of which further contributes to reprogramming. In certain embodiments described herein, reprogramming of a differentiated cell (e.g., a somatic cell) causes the differentiated cell to assume an undifferentiated state (e.g., is an undifferentiated cell). The resulting cells are referred to as “reprogrammed cells,” or “induced pluripotent stem cells (iPSCs or iPS cells).”

Reprogramming can involve alteration, e.g., reversal, of at least some of the heritable patterns of nucleic acid modification (e.g., methylation), chromatin condensation, epigenetic changes, genomic imprinting, etc., that occur during cellular differentiation. Reprogramming is distinct from simply maintaining the existing undifferentiated state of a cell that is already pluripotent or maintaining the existing less than fully differentiated state of a cell that is already a multipotent cell (e.g., a hematopoietic stem cell). Reprogramming is also distinct from promoting the self-renewal or proliferation of cells that are already pluripotent or multipotent, although the compositions and methods described herein can also be of use for such purposes, in some embodiments.

The specific approach or method used to generate pluripotent stem cells from somatic cells (broadly referred to as “reprogramming”) is not critical to the claimed invention. Thus, any method that re-programs a somatic cell to the pluripotent phenotype would be appropriate for use in the methods described herein.

Reprogramming methodologies for generating pluripotent cells using defined combinations of transcription factors have been described induced pluripotent stem cells. Yamanaka and Takahashi converted mouse somatic cells to ES cell-like cells with expanded developmental potential by the direct transduction of Oct4, Sox2, Klf4, and c-Myc (Takahashi and Yamanaka, 2006). iPSCs resemble ES cells as they restore the pluripotency-associated transcriptional circuitry and muc of the epigenetic landscape. In addition, mouse iPSCs satisfy all the standard assays for pluripotency: specifically, in vitro differentiation into cell types of the three germ layers, teratoma formation, contribution to chimeras, germline transmission (Maherali and Hochedlinger, 2008), and tetraploid complementation (Woltjen et al., 2009).

Subsequent studies have shown that human iPS cells can be obtained using similar transduction methods (Lowry et al., 2008; Park et al., 2008; Takahashi et al., 2007; Yu et al., 2007b), and the transcription factor trio, OCT4, SOX2, and NANOG, has been established as the core set of transcription factors that govern pluripotency (Jaenisch and Young, 2008). The production of iPS cells can be achieved by the introduction of nucleic acid sequences encoding stem cell-associated genes into an adult, somatic cell, historically using viral vectors.

iPS cells can be generated or derived from terminally differentiated somatic cells, as well as from adult stem cells, or somatic stem cells. That is, a non-pluripotent progenitor cell can be rendered pluripotent or multipotent by reprogramming. In such instances, it may not be necessary to include as many reprogramming factors as required to reprogram a terminally differentiated cell. Further, reprogramming can be induced by the non-viral introduction of reprogramming factors, e.g., by introducing the proteins themselves, or by introducing nucleic acids that encode the reprogramming factors, or by introducing messenger RNAs that upon translation produce the reprogramming factors (see e.g., Warren et al., Cell Stem Cell, 2010 Nov. 5; 7(5):618-30). Reprogramming can be achieved by introducing a combination of nucleic acids encoding stem cell-associated genes including, for example Oct-4 (also known as Oct-3/4 or Pouf51), Sox1, Sox2, Sox3, Sox 15, Sox 18, NANOG, Klf1, Klf2, Klf4, Klf5, NR5A2, c-Myc, 1-Myc, n-Myc, Rem2, Tert, and LIN28. In one embodiment, reprogramming using the methods and compositions described herein can further comprise introducing one or more of Oct-3/4, a member of the Sox family, a member of the Klf family, and a member of the Myc family to a somatic cell. In one embodiment, the methods and compositions described herein further comprise introducing one or more of each of Oct 4, Sox2, Nanog, c-MYC and Klf4 for reprogramming. As noted above, the exact method used for reprogramming is not necessarily critical to the methods and compositions described herein. However, where cells differentiated from the reprogrammed cells are to be used in, e.g., human therapy, in one embodiment the reprogramming is not effected by a method that alters the genome. Thus, in such embodiments, reprogramming is achieved, e.g., without the use of viral or plasmid vectors.

The efficiency of reprogramming (i.e., the number of reprogrammed cells) derived from a population of starting cells can be enhanced by the addition of various small molecules as shown by Shi, Y., et al (2008) Cell-Stem Cell 2:525-528, Huangfu, D., et al (2008) Nature Biotechnology 26(7):795-797, and Marson, A., et al (2008) Cell-Stem Cell 3:132-135. Thus, an agent or combination of agents that enhance the efficiency or rate of induced pluripotent stem cell production can be used in the production of patient-specific or disease-specific iPSCs. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), vitamin C, and trichostatin (TSA), among others.

Other non-limiting examples of reprogramming enhancing agents include: Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid ((VPA) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-Cl-UCHA (e.g., 6-(3-chlorophenylureido)caproic hydroxamic acid), AOE (2-amino-8-oxo-9,10-epoxydecanoic acid), CHAP31 and CHAP 50. Other reprogramming enhancing agents include, for example, dominant negative forms of the HDACs (e.g., catalytically inactive forms), siRNA inhibitors of the HDACs, and antibodies that specifically bind to the HDACs. Such inhibitors are available, e.g., from BIOMOL International, Fukasawa, Merck Biosciences, Novartis, Gloucester Pharmaceuticals, Aton Pharma, Titan Pharmaceuticals, Schering AG, Pharmion, MethylGene, and Sigma Aldrich.

To confirm the induction of pluripotent stem cells for use with the methods described herein, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, Fbx15, Ecat1, Esg1, Eras, Gdf3, Fgf4, Cripto, Dax1, Zpf296, Slc2a3, Rex1, Utf1, and Nat1. In one embodiment, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. In some embodiments, detection does not involve only RT-PCR, but also includes detection of protein markers. Intracellular markers may be best identified via RT-PCR, while cell surface markers are readily identified, e.g., by immunocytochemistry.

The pluripotent stem cell character of isolated cells can be confirmed by tests evaluating the ability of the iPSCs to differentiate to cells of each of the three germ layers. As one example, teratoma formation in nude mice can be used to evaluate the pluripotent character of the isolated clones. The cells are introduced to nude mice and histology and/or immunohistochemistry is performed on a tumor arising from the cells. The growth of a tumor comprising cells from all three germ layers, for example, further indicates that the cells are pluripotent stem cells.

### Somatic Cells for Reprogramming

Somatic cells, as that term is used herein, refer to any cells forming the body of an organism, excluding germline cells. Every cell type in the mammalian body—apart from the sperm and ova, the cells from which they are made (gametocytes) and undifferentiated stem cells—is a differentiated somatic cell. For example, internal organs, skin, bones, blood, and connective tissue are all made up of differentiated somatic cells.

Additional somatic cell types for use with the compositions and methods described herein include: a fibroblast (e.g., a primary fibroblast), a muscle cell (e.g., a myocyte), a cumulus cell, a neural cell, a mammary cell, a hepatocyte and a pancreatic islet cell. In some embodiments, the somatic cell is a primary cell line or is the progeny of a primary or secondary cell line. In some embodiments, the somatic cell is obtained from a human sample, e.g., a hair follicle, a blood sample, a biopsy (e.g., a skin biopsy or an adipose biopsy), a swab sample (e.g., an oral swab sample), and is thus a human somatic cell.

Some non-limiting examples of differentiated somatic cells include, but are not limited to, epithelial, endothelial, neuronal, adipose, cardiac, skeletal muscle, immune cells, hepatic, splenic, lung, circulating blood cells, gastrointestinal, renal, bone marrow, and pancreatic cells. In some embodiments, a somatic cell can be a primary cell isolated from any somatic tissue including, but not limited to brain, liver, gut, stomach, intestine, fat, muscle, uterus, skin, spleen, endocrine organ, bone, etc. Further, the somatic cell can be from any mammalian species, with non-limiting examples including a murine, bovine, simian, porcine, equine, ovine, or human cell. In some embodiments, the somatic cell is a human somatic cell.

When reprogrammed cells are used for generation of hematopoietic progenitor cells to be used in the therapeutic treatment of disease, it is desirable, but not required, to use somatic cells isolated from the patient being treated. For example, somatic cells involved in diseases, and somatic cells participating in therapeutic treatment of diseases and the like can be used. In some embodiments, a method for selecting the reprogrammed cells from a heterogeneous population comprising reprogrammed cells and somatic cells they were derived or generated from can be performed by any known means. For example, a drug resistance gene or the like, such as a selectable marker gene can be used to isolate the reprogrammed cells using the selectable marker as an index.

Reprogrammed somatic cells as disclosed herein can express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; β-III-tubulin; α-smooth muscle actin (α-SMA); fibroblast growth factor 4 (Fgf4), Cripto, DaxI; zinc finger protein 296 (Zfp296); N-acetyltransferase-1 (Nat1); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fthl17; Sal14; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; DPPA4; other general markers for pluripotency, etc. Other markers can include Dnmt3L; Sox15; Stat3; Grb2; β-catenin, and Bmi1. Such cells can also be characterized by the down-regulation of markers characteristic of the somatic cell from which the induced pluripotent stem cell is derived.

### Pharmaceutically Acceptable Carriers for Cells

The methods of administering human hematopoietic progenitor cells or genetic engineered cells described herein or their progeny to a subject as described herein involve the use of therapeutic compositions comprising hematopoietic progenitor cells. Therapeutic compositions contain a physiologically tolerable carrier together with the cell composition and optionally at least one additional bioactive agent as described herein, dissolved or dispersed therein as an active ingredient. In a preferred embodiment, the therapeutic composition is not substantially immunogenic when administered to a mammal or human patient for therapeutic purposes, unless so desired.

In general, the hematopoietic progenitor cells described herein or genetic engineered cells described herein or their progeny are administered as a suspension with a pharmaceutically acceptable carrier. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation comprising cells can include e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the hematopoietic progenitor cells as described herein using routine experimentation.

A cell composition can also be emulsified or presented as a liposome composition, provided that the emulsification procedure does not adversely affect cell viability. The cells and any other active ingredient can be mixed with excipients which are pharmaceutically acceptable and compatible with the active ingredient and in amounts suitable for use in the therapeutic methods described herein.

Additional agents included in a cell composition as described herein can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions as described herein that is effective in the treatment of a particular disorder or condition will depend on the nature of the disorder or condition, and can be determined by standard clinical techniques.

In some embodiments, the compositions of isolated genetic engineered cells described further comprises a pharmaceutically acceptable carrier. In one embodiment, the pharmaceutically acceptable carrier does not include tissue or cell culture media.

In some embodiments, the compositions of vectors described further comprises a pharmaceutically acceptable carrier. In one embodiment, the pharmaceutically acceptable carrier does not include tissue or cell culture media.

In some embodiments, the compositions of RNPs described further comprises a pharmaceutically acceptable carrier. In one embodiment, the pharmaceutically acceptable carrier does not include tissue or cell culture media.

### Administration & Efficacy

As used herein, the terms “administering,” “introducing” and “transplanting” are used interchangeably in the context of the placement of cells, e.g. hematopoietic progenitor cells, vectors, RNPs or compositions thereof, as described herein into a subject, by a method or route which results in at least partial localization of the introduced agent (i.e., hematopoietic progenitor cells or engineered cells with reduced BCL11A expression, vector, RNP or composition described herein) at a desired site, such as a site of injury or repair, such that a desired effect(s) is produced. The cells, e.g. hematopoietic progenitor cells, or their differentiated progeny can be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years, i.e., long-term engraftment. For example, in some embodiments of the aspects described herein, an effective amount of hematopoietic progenitor cells or engineered cells with reduced BCL11A expression is administered via a systemic route of administration, such as an intraperitoneal or intravenous route. The vectors, RNP or compositions described herein be administered by any appropriate route which results in its delivery to a desired location in the subject.

When provided prophylactically, agent (i.e., hematopoietic progenitor cells or engineered cells with reduced BCL11A expression, vector, RNP or composition described herein) can be administered to a subject in advance of any symptom of a hemoglobinopathy, e.g., prior to the switch from fetal γ-globin to predominantly β-globin. Accordingly, the prophylactic administration of a hematopoietic progenitor cell population serves to prevent a hemoglobinopathy, as disclosed herein.

When provided therapeutically, the agent is provided at (or after) the onset of a symptom or indication of a hemoglobinopathy, e.g., upon the onset of sickle cell disease.

In some embodiments of the aspects described herein, the hematopoietic progenitor cell population or engineered cells with reduced BCL11A expression being administered according to the methods described herein comprises allogeneic hematopoietic progenitor cells obtained from one or more donors. As used herein, “allogeneic” refers to a hematopoietic progenitor cell or biological samples comprising hematopoietic progenitor cells obtained from one or more different donors of the same species, where the genes at one or more loci are not identical. For example, a hematopoietic progenitor cell population or engineered cells with reduced BCL11A expression being administered to a subject can be derived from umbilical cord blood obtained from one more unrelated donor subjects, or from one or more non-identical siblings. In some embodiments, syngeneic hematopoietic progenitor cell populations can be used, such as those obtained from genetically identical animals, or from identical twins. In other embodiments of this aspect, the hematopoietic progenitor cells are autologous cells; that is, the hematopoietic progenitor cells are obtained or isolated from a subject and administered to the same subject, i.e., the donor and recipient are the same.

For use in the various aspects described herein, an effective amount of hematopoietic progenitor cells or engineered cells with reduced BCL11A expression, comprises at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ hematopoietic progenitor cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The hematopoietic progenitor cells or engineered cells with reduced BCL11A expression can be derived from one or more donors, or can be obtained from an autologous source. In some embodiments of the aspects described herein, the hematopoietic progenitor cells are expanded in culture prior to administration to a subject in need thereof.

In one embodiment, the term “effective amount” as used herein refers to the amount of an agent (i.e., hematopoietic progenitor cells or engineered cells with reduced BCL11A expression, vector, RNP or composition described herein) needed to alleviate at least one or more symptom of a hemoglobinopathy, and relates to a sufficient amount of a composition to provide the desired effect, e.g., treat a subject having a hemoglobinopathy. The term “therapeutically effective amount” therefore refers to an amount of an agent that is sufficient to promote a particular effect when administered to a typical subject, such as one who has or is at risk for a hemoglobinopathy. An effective amount as used herein would also include an amount sufficient to prevent or delay the development of a symptom of the disease, alter the course of a symptom disease (for example but not limited to, slow the progression of a symptom of the disease), or reverse a symptom of the disease. It is understood that for any given case, an appropriate “effective amount” can be determined by one of ordinary skill in the art using routine experimentation.

As used herein, “administered” refers to the delivery of an agent (i.e., hematopoietic progenitor cells or engineered cells with reduced BCL11A expression, vector, RNP or composition described herein) as described herein into a subject by a method or route which results in at least partial localization of the agent composition at a desired site. An agent can be administered by any appropriate route which results in effective treatment in the subject, i.e. administration results in delivery to a desired location in the subject where at least a portion of the composition delivered, i.e. at least 1×10⁴ cells are delivered to the desired site for a period of time. Modes of administration include injection, infusion, instillation, or ingestion. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebrospinal, and intrasternal injection and infusion. For the delivery of cells, administration by injection or infusion is generally preferred.

In one embodiment, the agent (i.e., hematopoietic progenitor cells or engineered cells with reduced BCL11A expression, vector, RNP or composition described herein) as described herein is administered systemically. The phrases “systemic administration,” “administered systemically”, “peripheral administration” and “administered peripherally” as used herein refer to the administration of a population of hematopoietic progenitor cells other than directly into a target site, tissue, or organ, such that it enters, instead, the subject's circulatory system and, thus, is subject to metabolism and other like processes.

The efficacy of a treatment comprising an agent (i.e., hematopoietic progenitor cells or engineered cells with reduced BCL11A expression, vector, RNP or composition described herein) as described herein for the treatment of a hemoglobinopathy can be determined by the skilled clinician. However, a treatment is considered “effective treatment,” as the term is used herein, if any one or all of the signs or symptoms of, as but one example, levels of fetal β-globin are altered in a beneficial manner, other clinically accepted symptoms or markers of disease are improved or ameliorated, e.g., by at least 10% following treatment with an agent. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of sepsis; or (2) relieving the disease, e.g., causing regression of symptoms; and (3) preventing or reducing the likelihood of the development of infection or sepsis.

The treatment according to the present invention ameliorates one or more symptoms associated with a β-globin disorder by increasing the amount of fetal hemoglobin in the individual. Symptoms typically associated with a hemoglobinopathy, include for example, anemia, tissue hypoxia, organ dysfunction, abnormal hematocrit values, ineffective erythropoiesis, abnormal reticulocyte (erythrocyte) count, abnormal iron load, the presence of ring sideroblasts, splenomegaly, hepatomegaly, impaired peripheral blood flow, dyspnea, increased hemolysis, jaundice, anemic pain crises, acute chest syndrome, splenic sequestration, priapism, stroke, hand-foot syndrome, and pain such as angina pectoris.

In one embodiment, any method known in the art can be used to measure an increase in fetal hemoglobin expression, e.g., Western Blot analysis of fetal hemoglobin protein and quantifying mRNA of fetal γ-globin.

This invention is further illustrated by the following example which should not be construed as limiting. The contents of all references cited throughout this application, as well as the figures and table are incorporated herein by reference.

The invention described herein can be further described in any of the numbered paragraphs provided herein below:
1. A vector comprising at least two guide RNAs (gRNAs) that targets and hybridizes to a target sequence on a DNA molecule, wherein each of the at least two gRNAs hybridizes at least two genomic DNA locations selected from human chromosome 2 at location 60725424 to 60725688 according to GRCh37/hg19 human genome reference build (+55 functional region); human chromosome 2 at location 60722238 to 60722466 GRCh37/hg19 human genome reference build (+58 functional region); or human chromosome 2 at location 60718042 to 60718186 according to GRCh37/hg19 human genome reference build (+62 functional region).
2. The vector of paragraph 1, wherein the gRNA has a selected from the group consisting of
#1617: CUAACAGUUGCUUUUAUCAC (SEQ ID NO: 1),
#1618: UUGCUUUUAUCACAGGCUCC (SEQ ID NO: 2),
#1449: GUGCUUGGUCGGCACUGAUA (SEQ ID NO: 3), and
#1450: UGCUUGGUCGGCACUGAUAG (SEQ ID NO: 4).
3. The vector of any of the preceding paragraphs, further comprising a DNA endonuclease enzyme.
4. A ribonucleoprotein (RNP) complex comprising a DNA endonuclease enzyme and at least two guide RNAs (gRNAs) that targets and hybridizes to a target sequence on a DNA molecule, wherein each of the at least two gRNAs hybridizes at least two genomic DNA locations selected from human chromosome 2 at location 60725424 to 60725688 according to GRCh37/hg19 human genome reference build (+55 functional region); human chromosome 2 at location 60722238 to 60722466 GRCh37/hg19 human genome reference build (+58 functional region); or human chromosome 2 at location 60718042 to 60718186 according to GRCh37/hg19 human genome reference build (+62 functional region).
5. The RNP of any of the preceding paragraphs, wherein the at least two gRNAs hybridize to the same genomic DNA location.
6. The RNP of any of the preceding paragraphs, wherein the at least two gRNAs hybridize to different genomic DNA locations.
7. The RNP of any of the preceding paragraphs, wherein the RNP has one gRNA that hybridizes to the +55 functional region and one gRNA that hybridizes to the +58 functional region.
8. The RNP of any of the preceding paragraphs, wherein the RNP has one gRNA that hybridizes to the +55 functional region and one gRNA that hybridizes to the +62 functional region 1.
9. The RNP of any of the preceding paragraphs, wherein the RNP has one gRNA that hybridizes to the +58 functional region and one gRNA that hybridizes to the +62 functional region.
10. The RNP of any of the preceding paragraphs, wherein the gRNA has a selected from the group consisting of #1617: CUAACAGUUGCUUUUAUCAC (SEQ ID NO: 1),
#1618: UUGCUUUUAUCACAGGCUCC (SEQ ID NO: 2),
#1449: GUGCUUGGUCGGCACUGAUA (SEQ ID NO: 3), and
#1450: UGCUUGGUCGGCACUGAUAG (SEQ ID NO: 4).
11. The RNP of any of the preceding paragraphs, wherein the DNA endonuclease comprises at least one nuclear localization signal (NLS) sequence fused at or near its amino terminus.
12. The RNP of any of the preceding paragraphs, wherein the DNA endonuclease further comprises at least one NLS sequence fused at or near its carboxy terminus.
13. The RNP of any of the preceding paragraphs, wherein the DNA endonuclease has one NLS sequence fused at or near its amino terminus, and two nuclear localization signal sequences fused at or near its carboxy terminus.
14. The RNP of any of the preceding paragraphs, wherein the NLS sequence is selected from the group consisting of: SV40 large T-antigen, nucleoplasmin, c-Myc, c-Myc-like, and hRNPA1.
15. The RNP of any of the preceding paragraphs, wherein the at least one nuclear localization signal sequences are identical.
16. The RNP of any of the preceding paragraphs, wherein the at least one nuclear localization signal sequences are different.
17. The RNP of any of the preceding paragraphs, wherein the DNA endonuclease has a c-Myc-like nuclear localization signal sequence fused to its amino terminus and an SV40 nuclear localization signal sequence and nucleplasmin bipartate nuclear localization signal sequence fused to its carboxyl terminus.
18. The RNP of any of the preceding paragraphs, wherein the DNA endonuclease is selected from the group consisting of a Zinc Finger Nuclease (ZFN), a Transcription Activator-Like Effector Nuclease (TALENs), and a CRISPR-associated protein (Cas) nuclease.
19. The RNP of any of the preceding paragraphs, wherein the CRISPR enzyme is a type II CRISPR system enzyme.
20. The RNP of any of the preceding paragraphs, wherein the CRISPR enzyme is a Cas protein.
21. The RNP of any of the preceding paragraphs, wherein the Cas protein is selected from the group consisting of: Cpf1, C2c1, C2c3, Cas12a, Cas12b, Cas12c, Cas12d, Cas12e, Cas13a, Cas13b, and Cas13c. Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c.
22. The RNP complex of any of the preceding paragraphs for the use of altering the expression of at least one gene product.
23. A vector comprising the RNP of any of the preceding paragraphs.
24. A composition comprising the vector of any of the preceding paragraphs, RNP of any of the preceding paragraphs or vector of any of the preceding paragraphs.
25. A pharmaceutical composition of comprising the vector of any of the preceding paragraphs, RNP of any of the preceding paragraphs or vector of any of the preceding paragraphs.
26. A method for producing a progenitor cell having decreased BCL11A mRNA or protein expression, the method comprising contacting an isolated progenitor cell with a vector of any of the preceding paragraphs, RNP of any of the preceding paragraphs, a vector of any of the preceding paragraphs, or a composition of any of the preceding paragraphs.
27. A method for producing an isolated genetic engineered human cell having at least one genetic modification comprising contacting an isolated cell with an effective amount of a vector of any of the preceding paragraphs, RNP of any of the preceding paragraphs, a vector of any of the preceding paragraphs, or a composition of any of the preceding paragraphs, causing at least one genetic modification therein.
28. The method of any of the preceding paragraphs, wherein the isolated progenitor cell or isolated cell is a hematopoietic progenitor cell.
29. The method of any of the preceding paragraphs, wherein the hematopoietic progenitor is a cell of the erythroid lineage.
30. The method of any of the preceding paragraphs, wherein the isolated progenitor cell or isolated cell is an induced pluripotent stem cell.
31. The method of any of the preceding paragraphs, wherein the hematopoietic progenitor cell is contacted ex vivo or in vitro.
32. The method of any of the preceding paragraphs, wherein the at least one genetic modification is an inversion, an insertion or a deletion.
33. A composition comprising progenitor cells obtained by the method of any of the preceding paragraphs.
34. A composition comprising isolated genetic engineered human cells obtained by the method of any of the preceding paragraphs.
35. A method of increasing fetal hemoglobin levels in a cell, the method comprising the steps of: contacting an isolated cell with an effective amount of a composition comprising at a vector of any of the preceding paragraphs, RNP of any of the preceding paragraphs, a vector of any of the preceding paragraphs, or a composition of any of the preceding paragraphs, whereby fetal hemoglobin expression is increased in said cell, or its progeny, relative to said cell prior to said contacting.
36. The method of any of the preceding paragraphs, wherein the isolated cell is a hematopoietic progenitor cell.
37. The method of any of the preceding paragraphs, wherein the hematopoietic progenitor cell is a cell of the erythroid lineage.
38. The method of any of the preceding paragraphs, wherein the isolated cell is an induced pluripotent stem cell.
39. The method of any of the preceding paragraphs, wherein the hematopoietic progenitor cell is contacted ex vivo or in vitro.
40. The method of any of the preceding paragraphs, wherein the at least one genetic modification is an inversion, an insertion or a deletion.
41. A method for increasing fetal hemoglobin levels in a mammal in need thereof, the method comprising the steps of contacting an isolated hematopoietic progenitor cell in said mammal with an effective amount of a composition comprising a vector of any of the preceding paragraphs, RNP of any of the preceding paragraphs, a vector of any of the preceding paragraphs, or a composition of any of the preceding paragraphs, causing at least one genetic modification therein, whereby fetal hemoglobin expression is increased in said mammal, relative to expression prior to said contacting.
42. A method for increasing fetal hemoglobin levels in a mammal in need thereof, the method comprising transplanting an isolated genetic engineered human cell of any of the preceding paragraphs or a composition of any of the preceding paragraphs into the mammal.

### Example 1

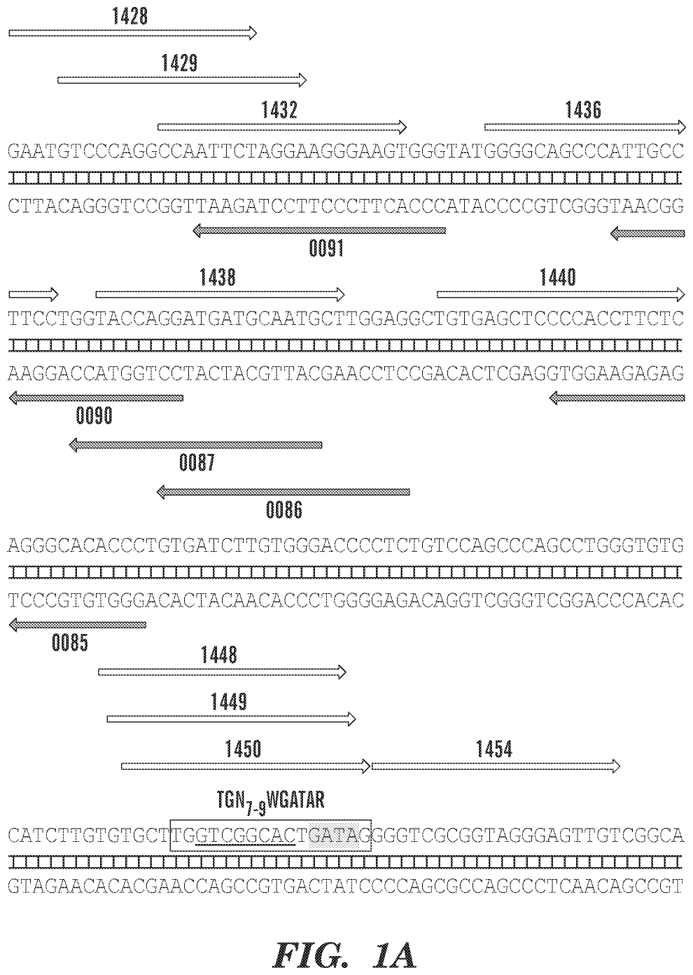

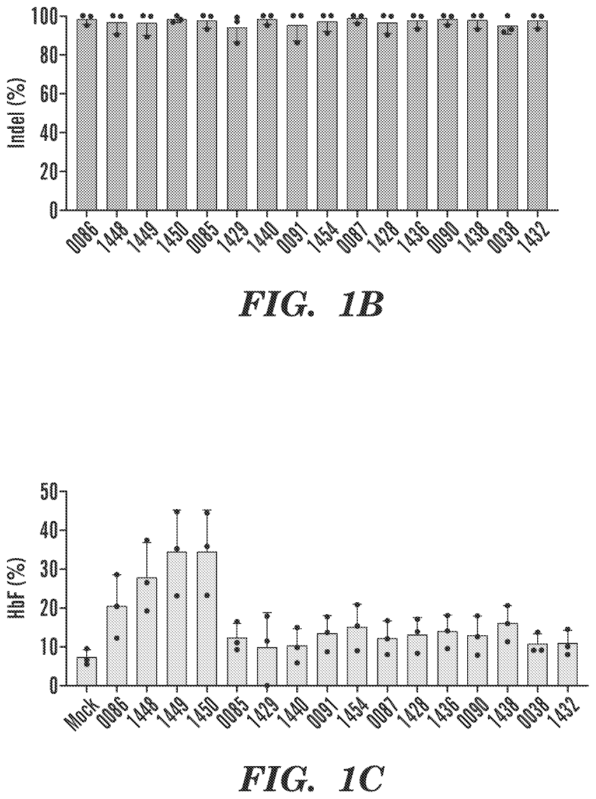

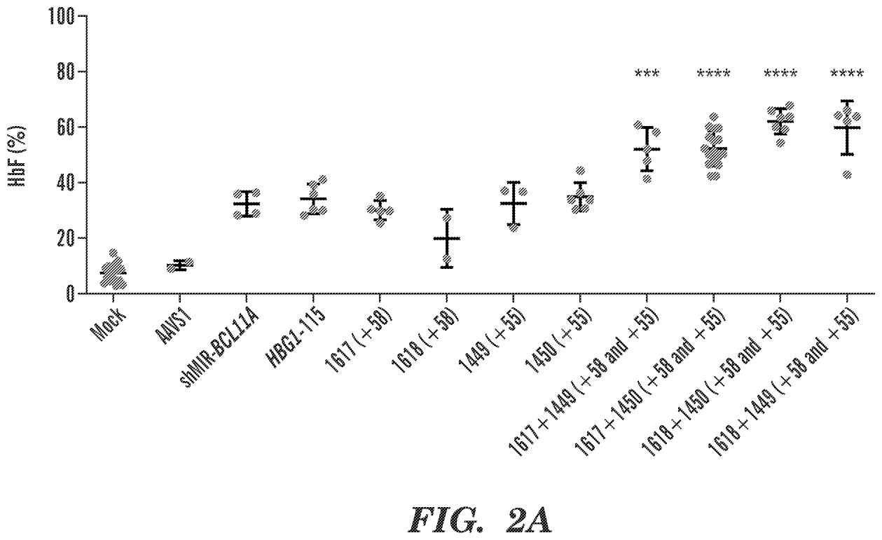

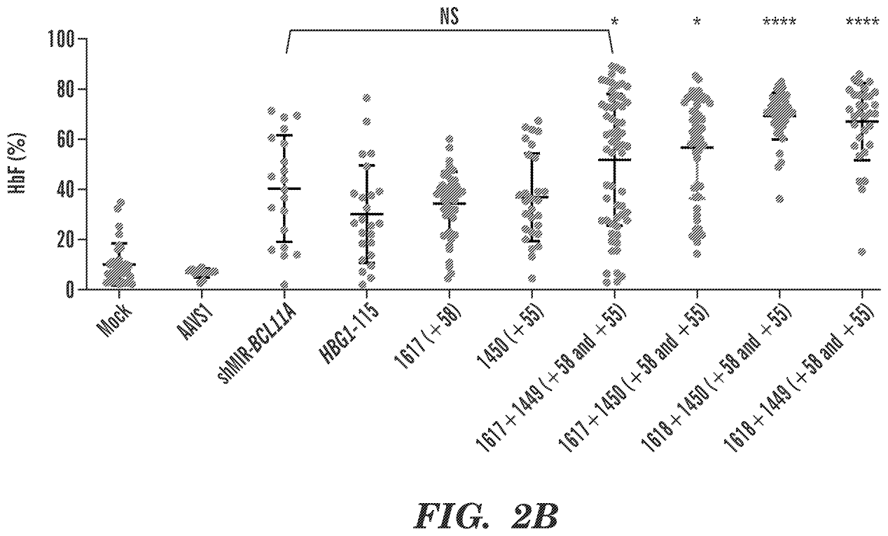

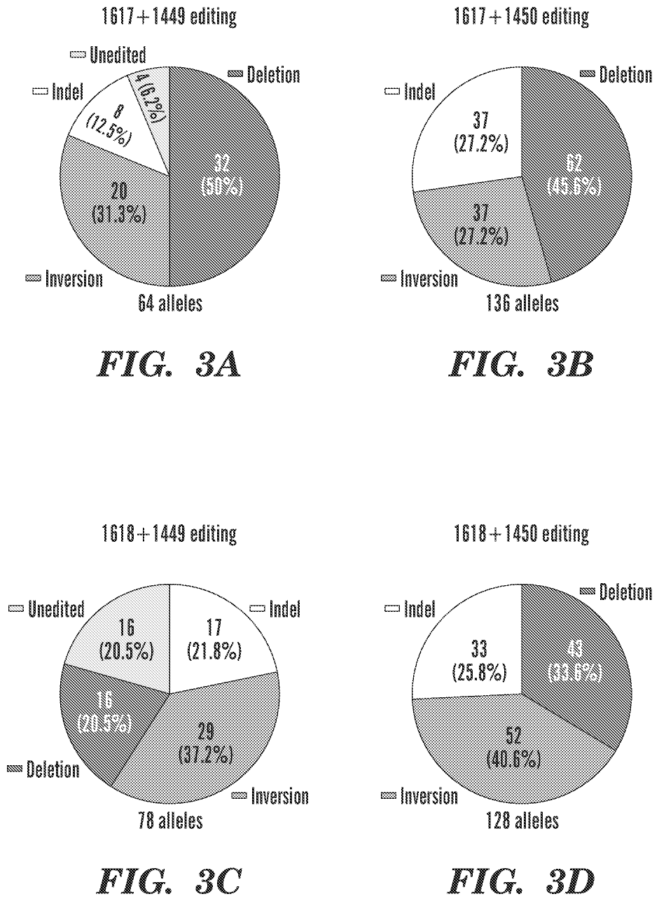

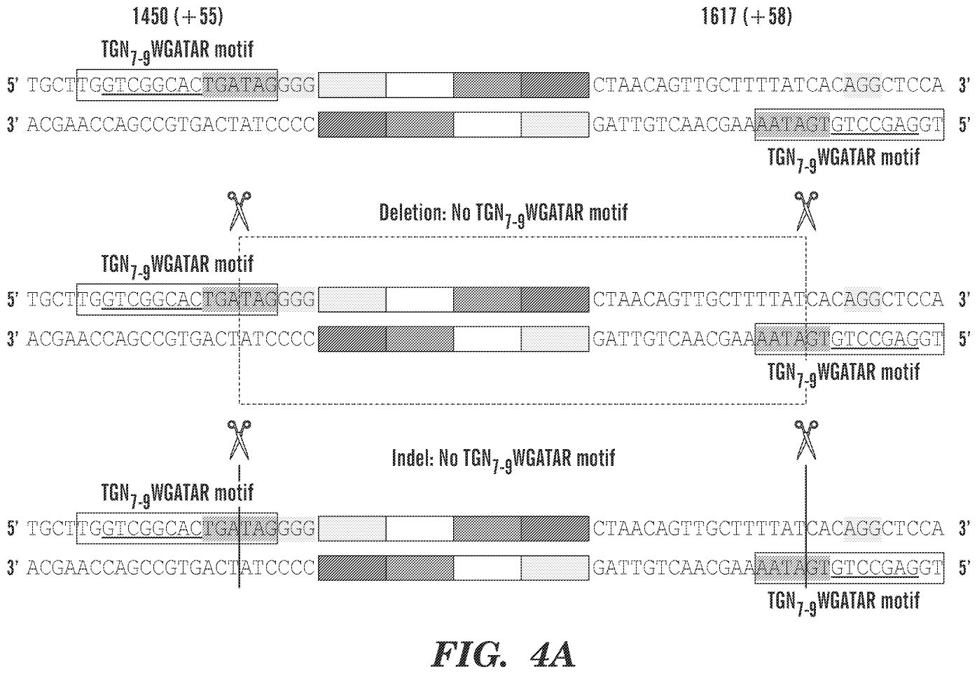

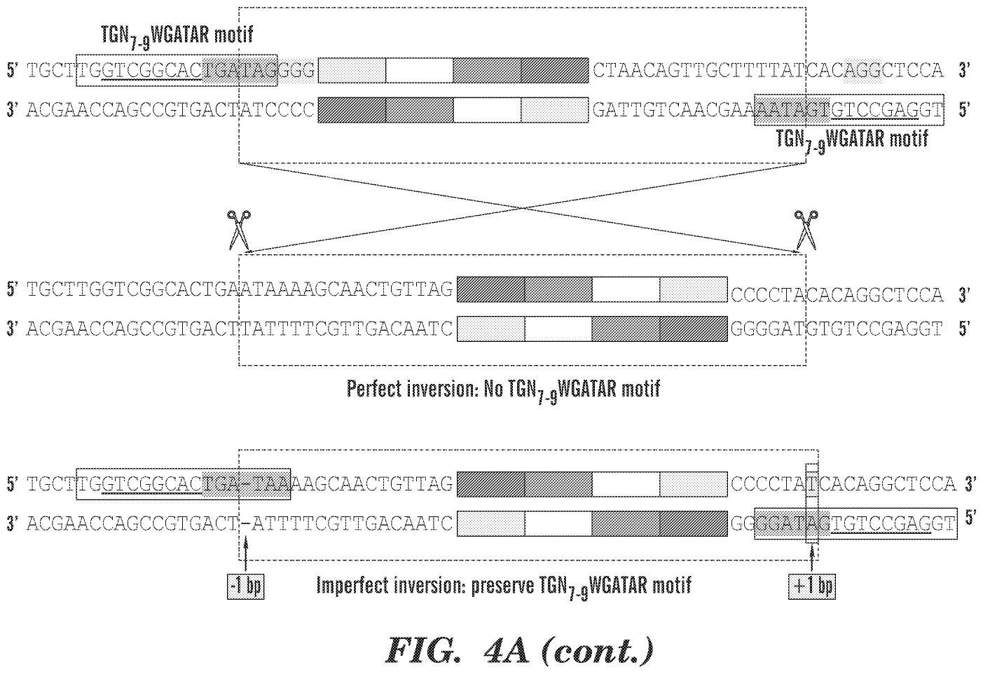

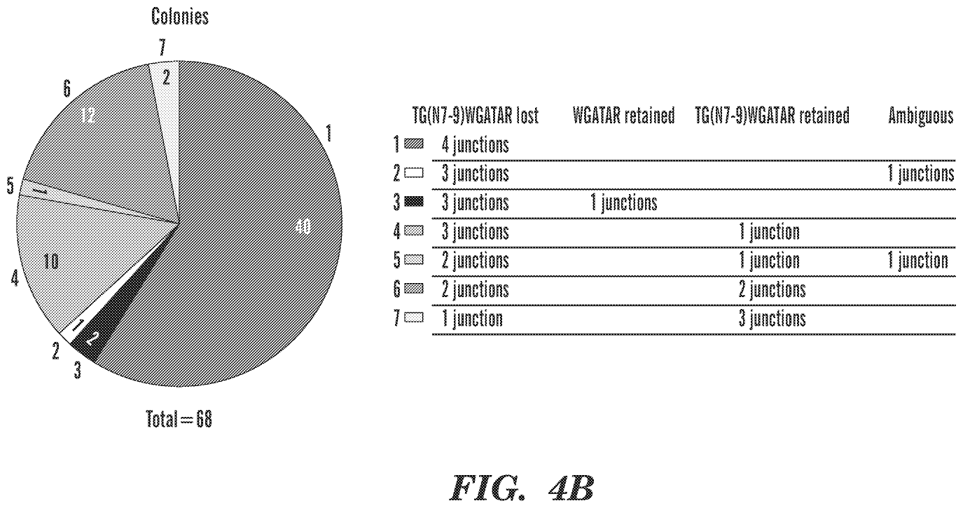

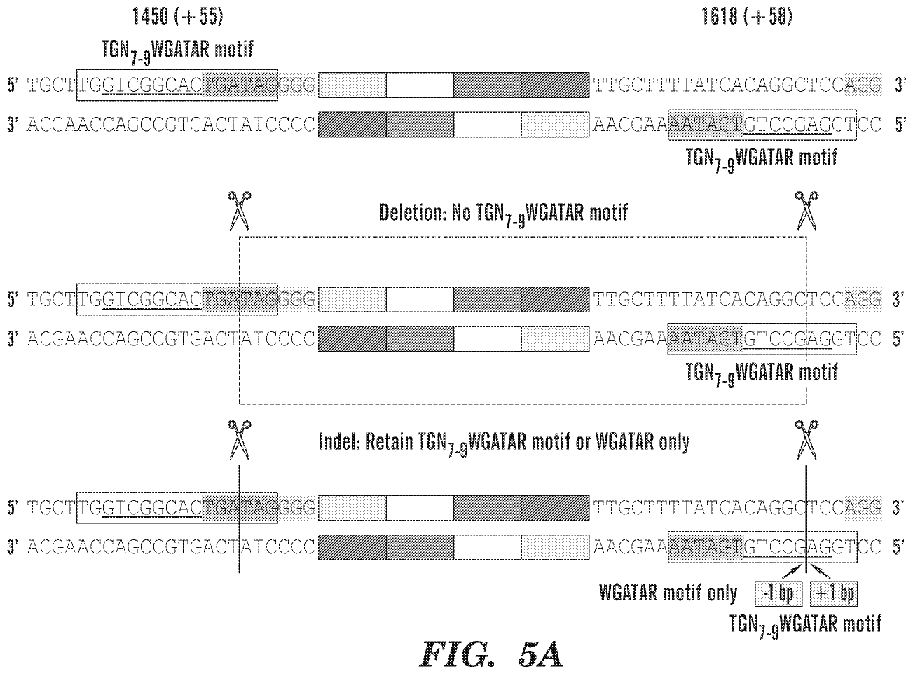

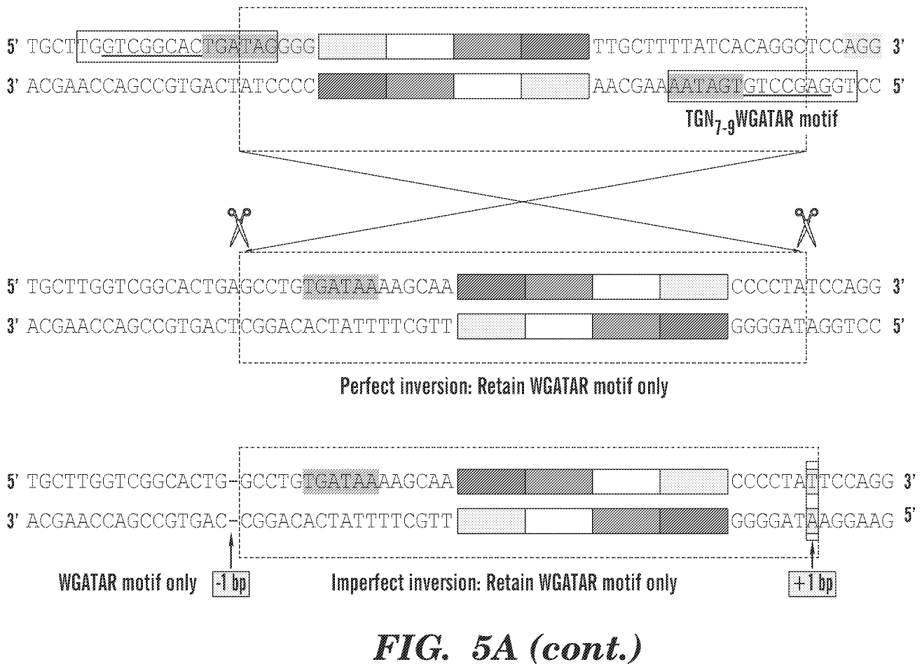

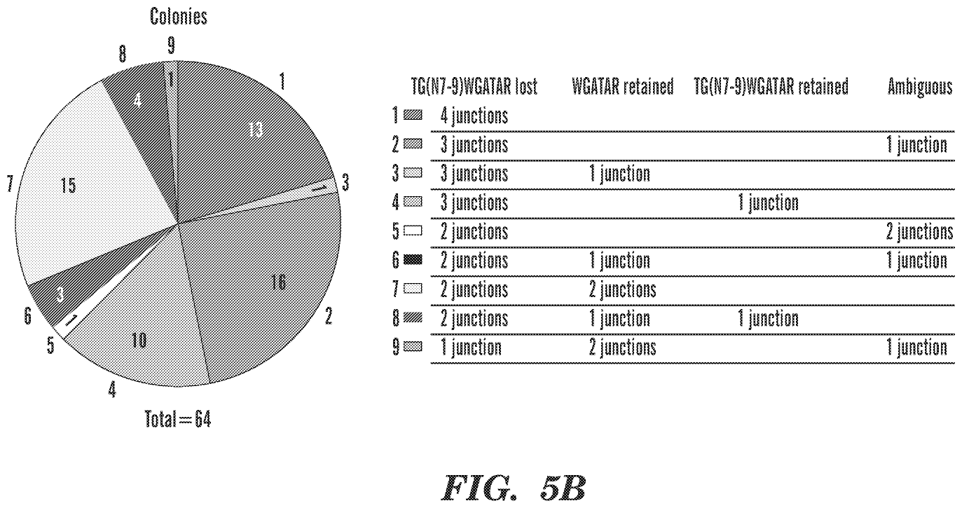

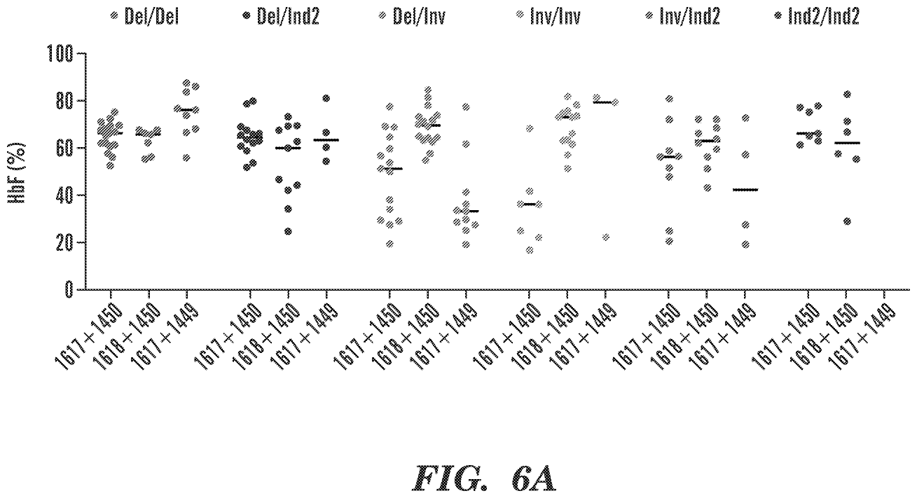

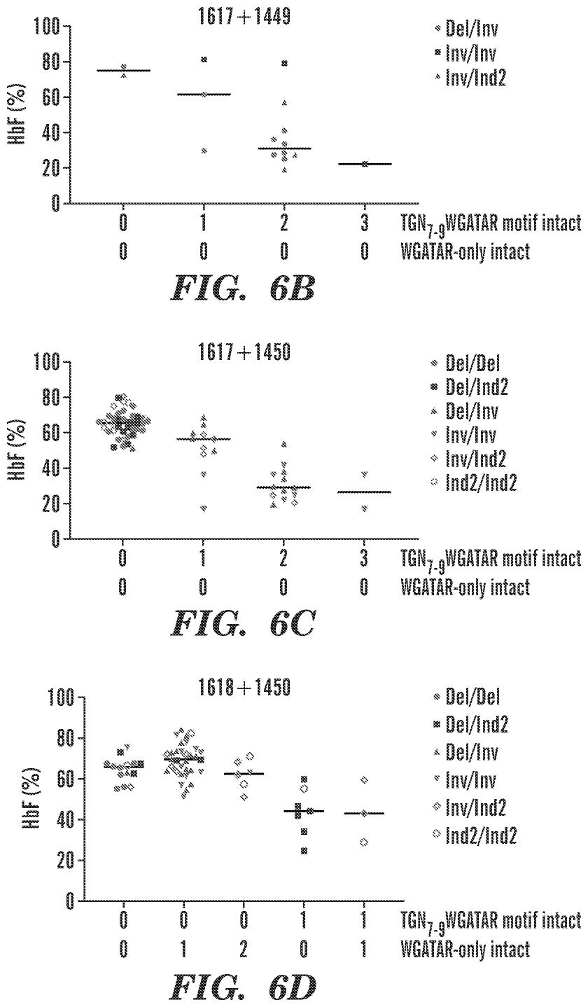

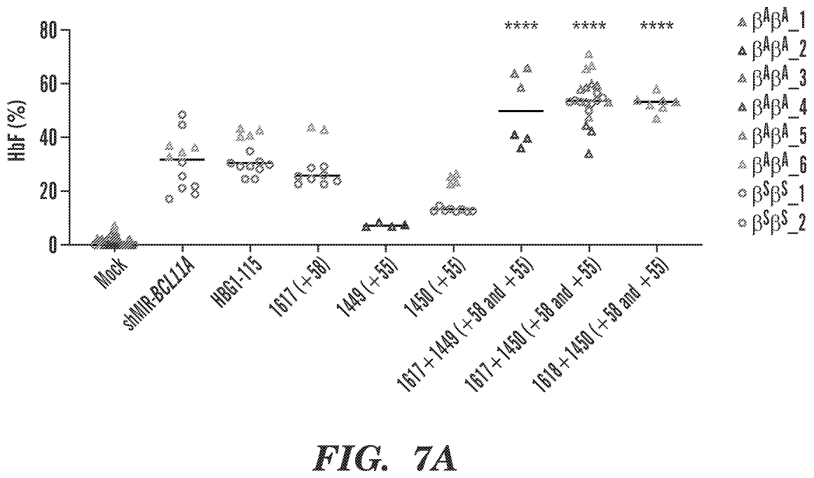

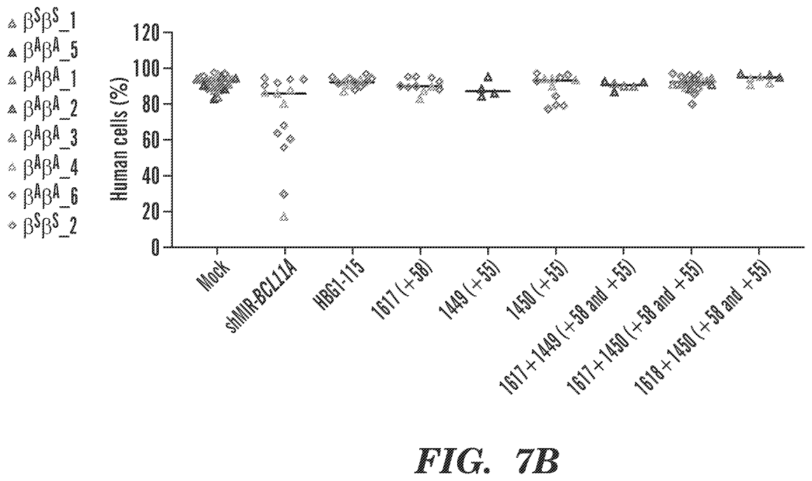

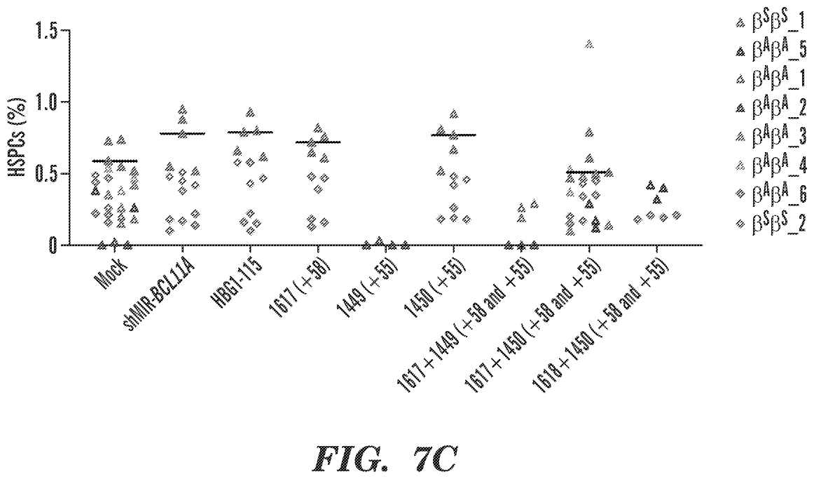

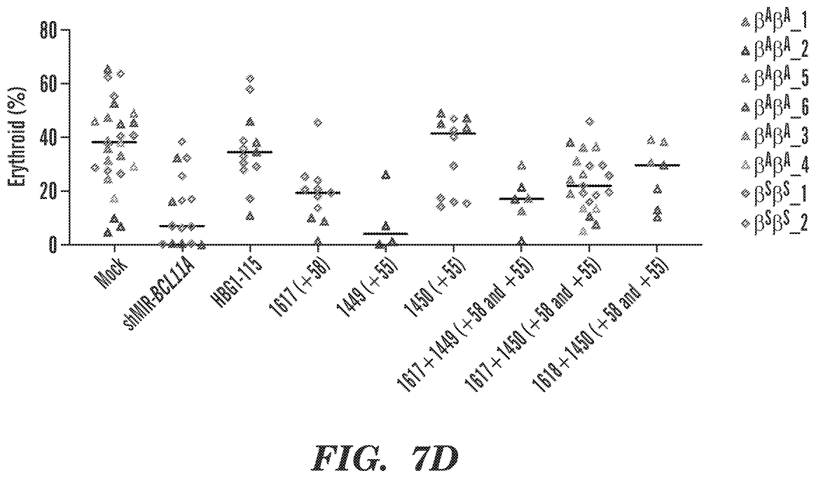

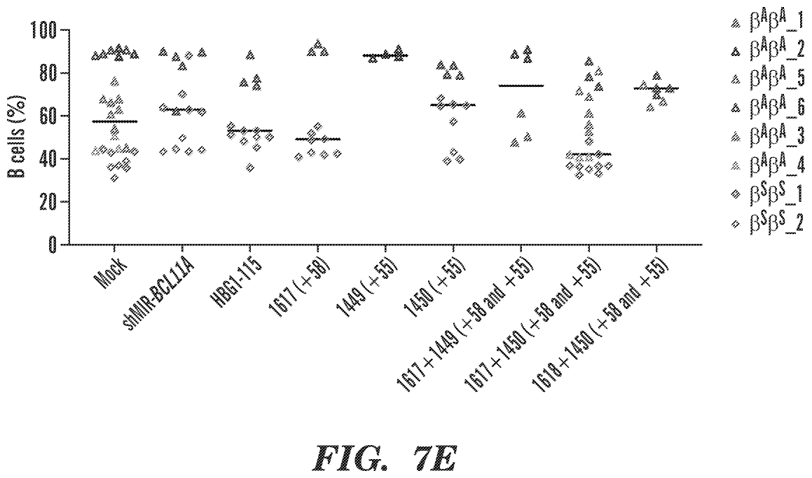

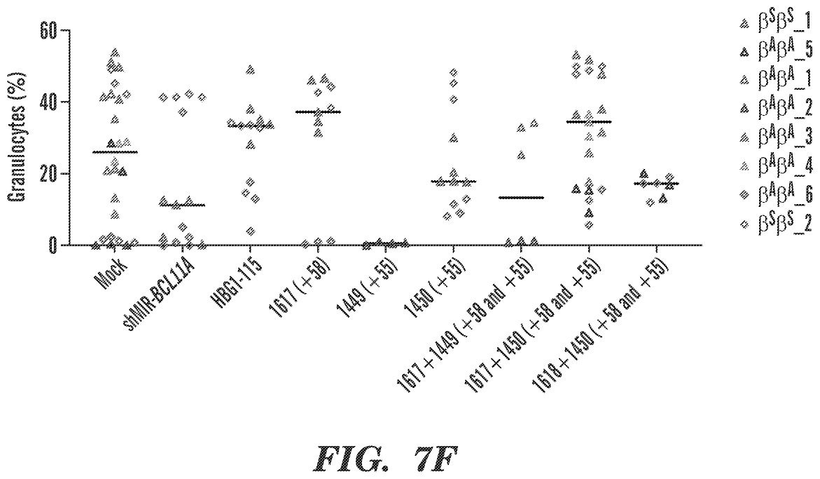

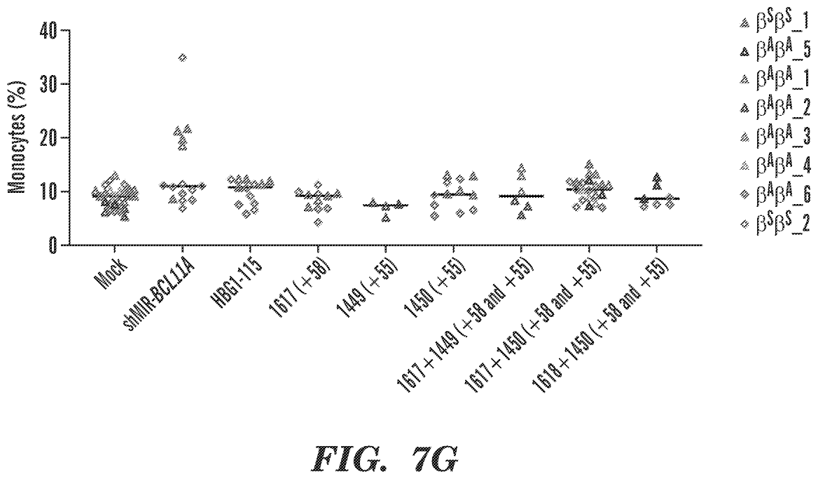

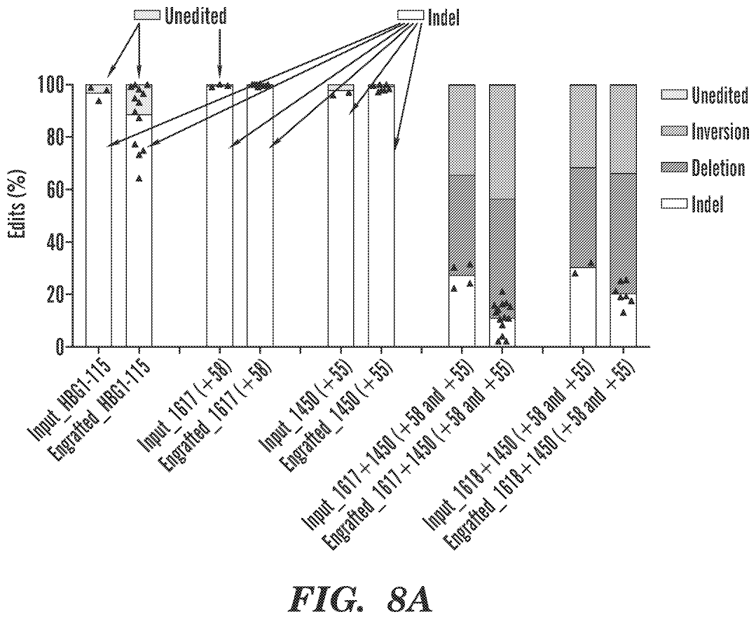

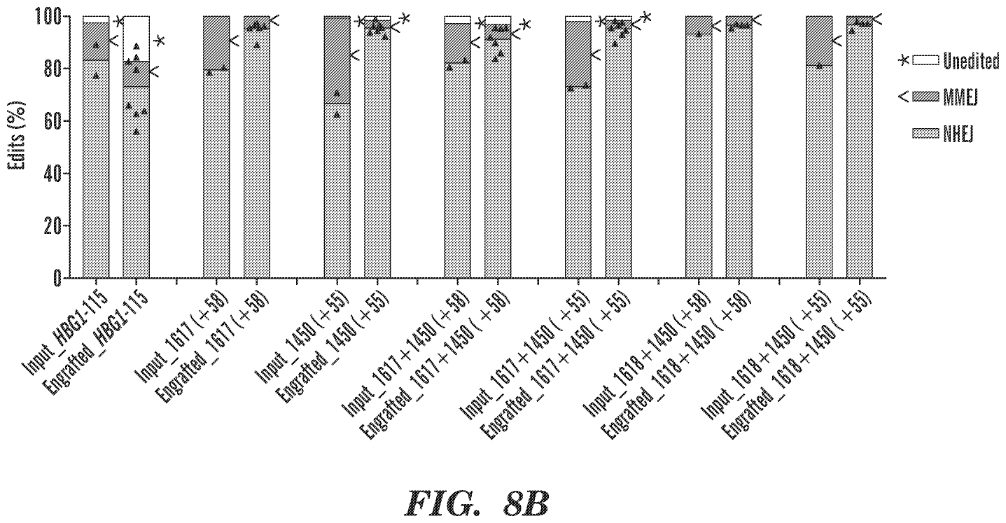

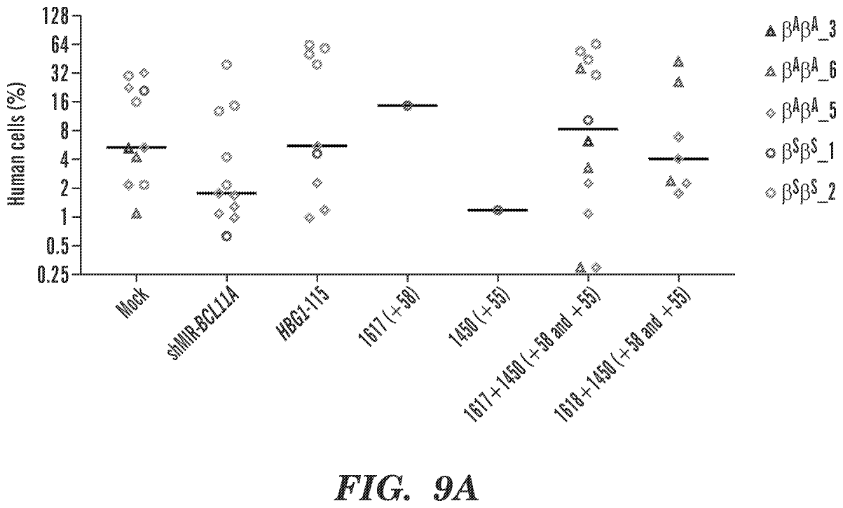

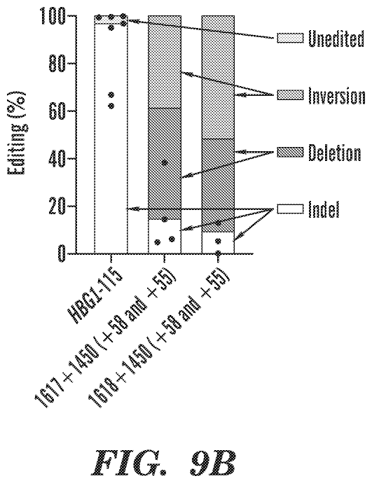

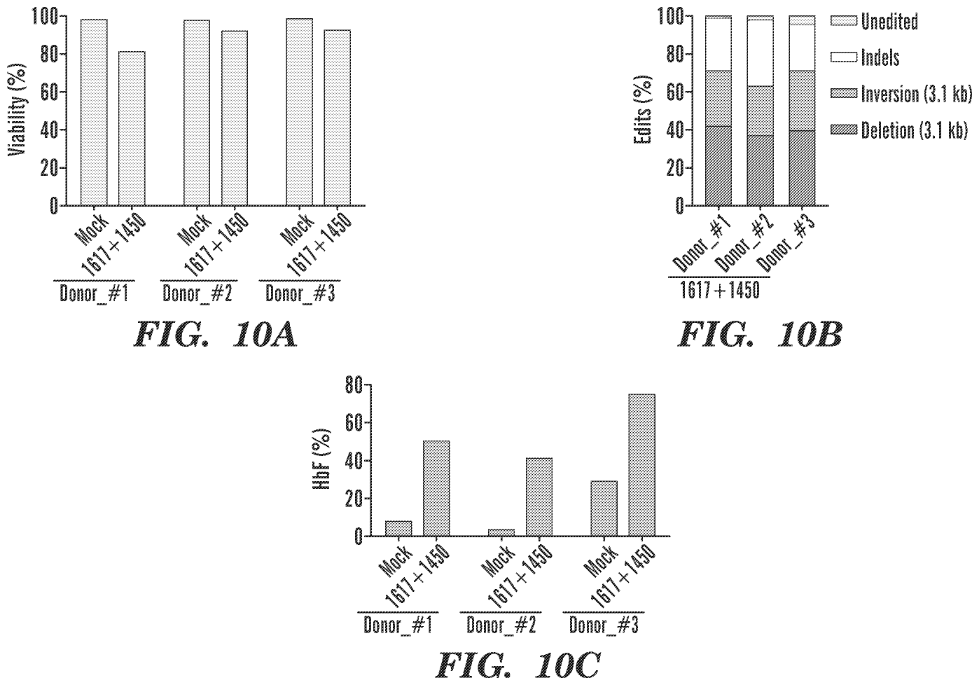

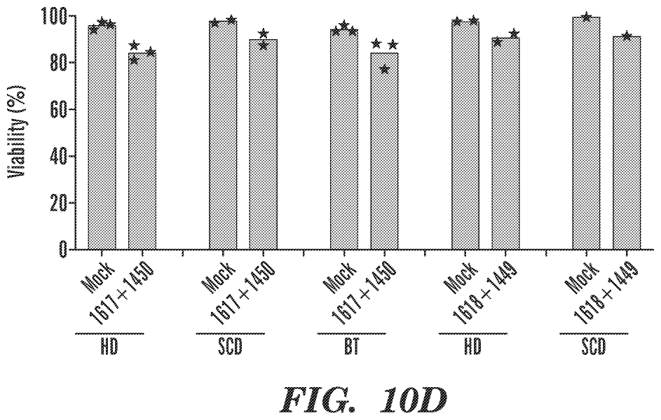

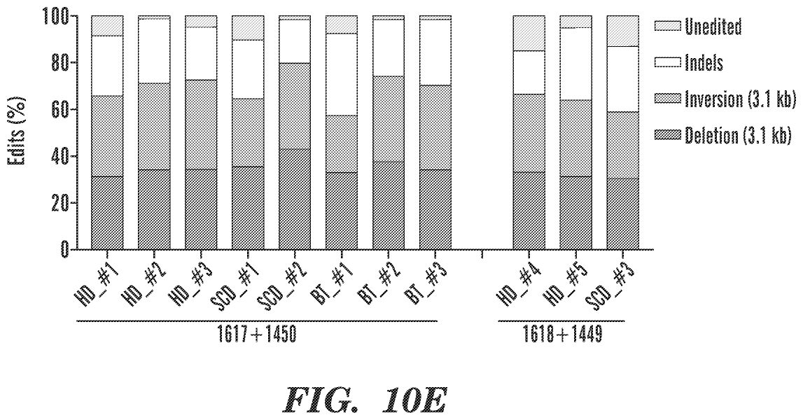

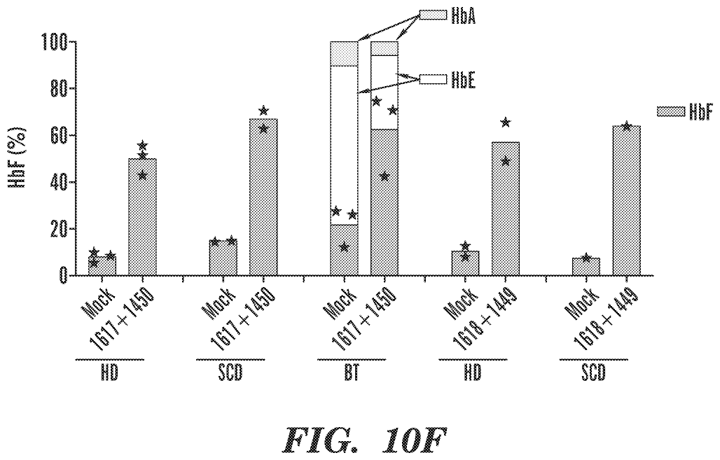

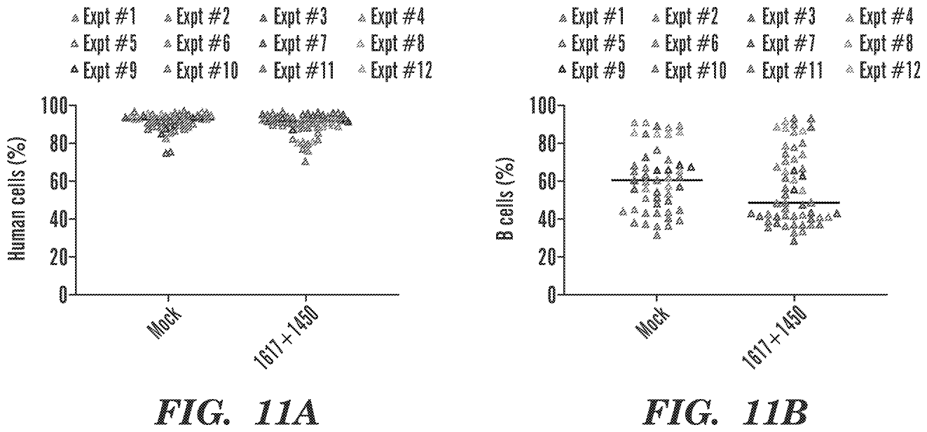

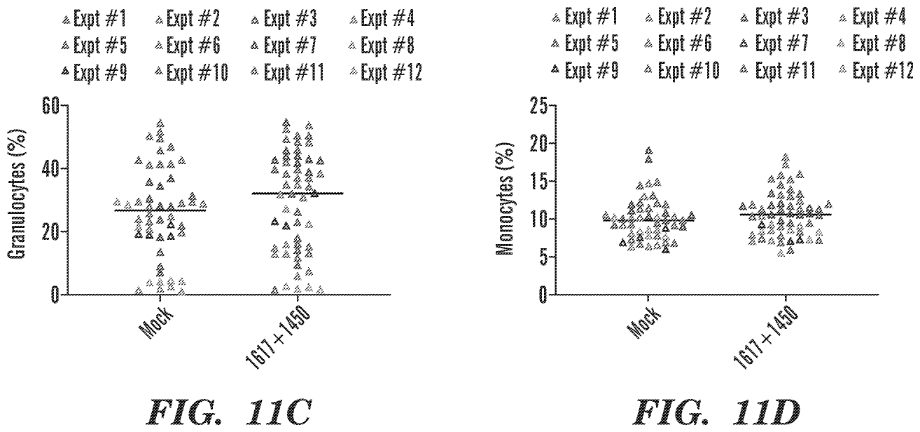

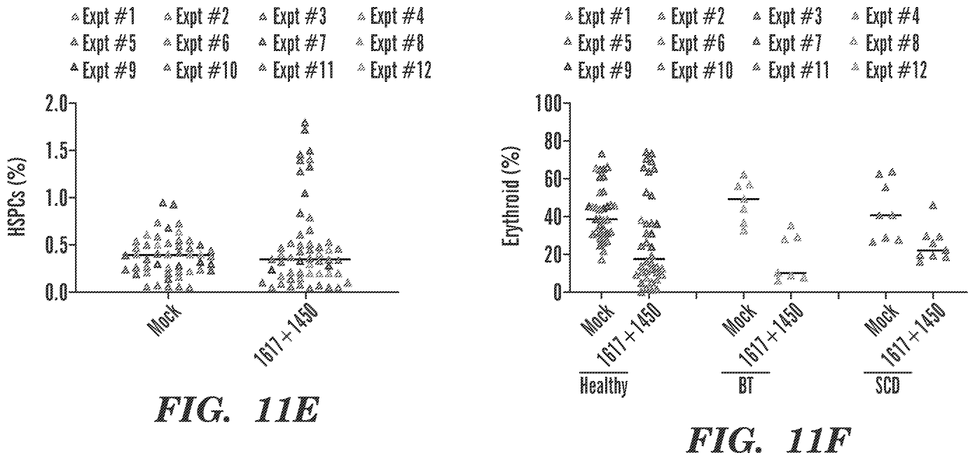

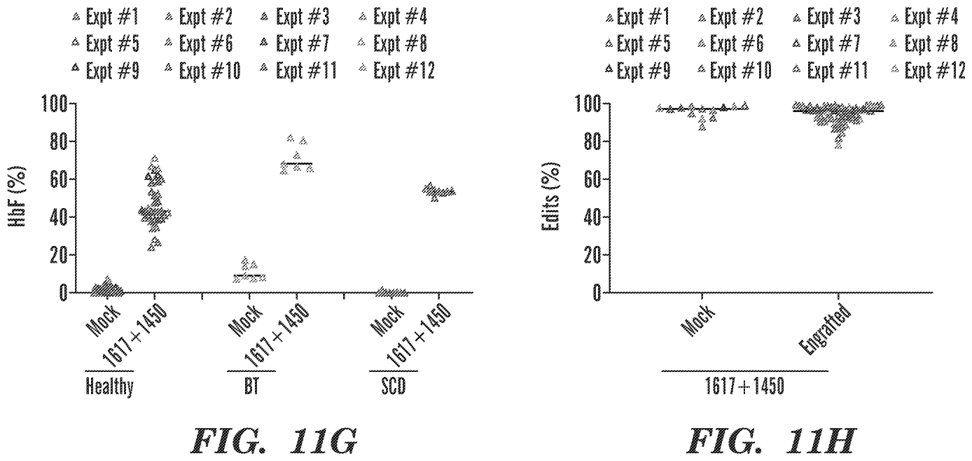

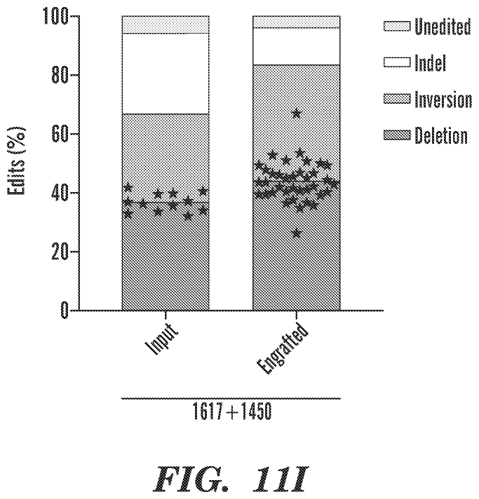

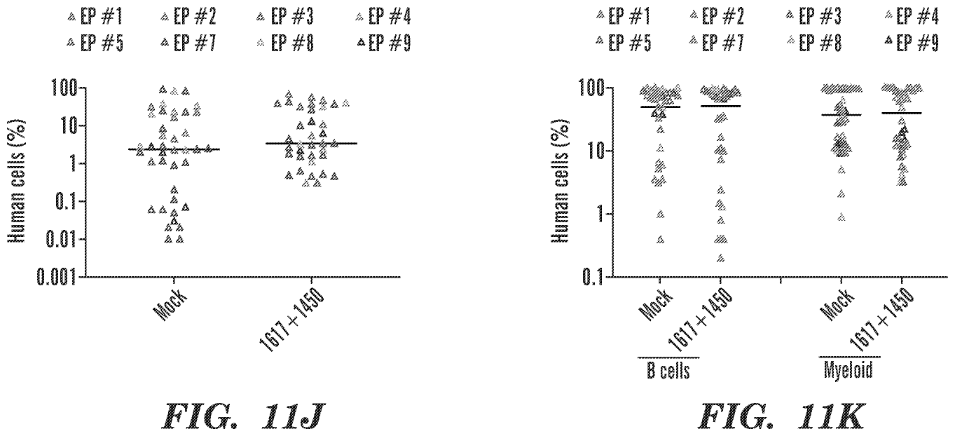

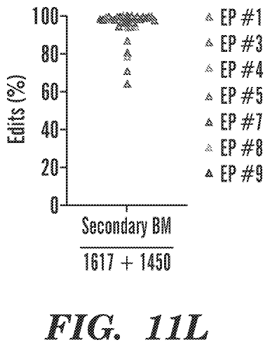

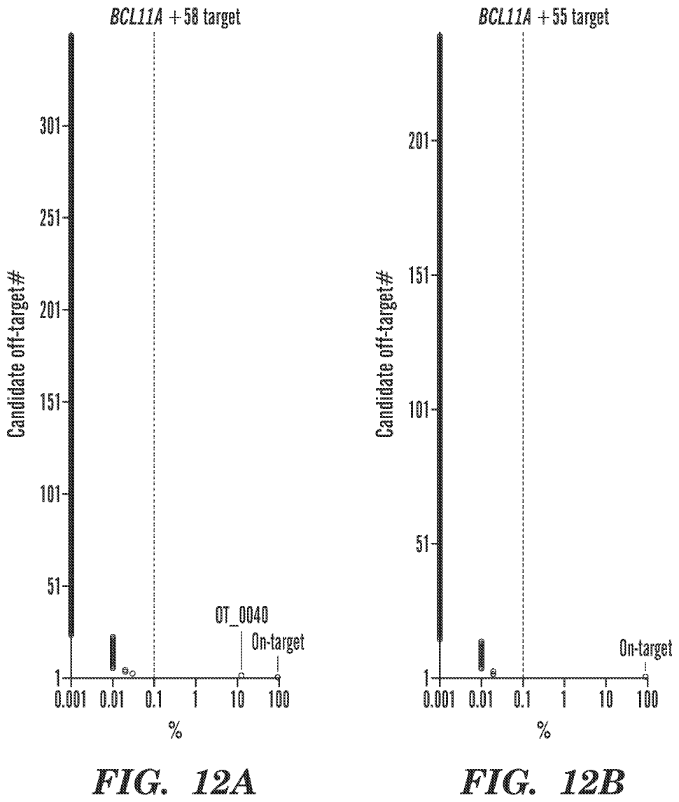

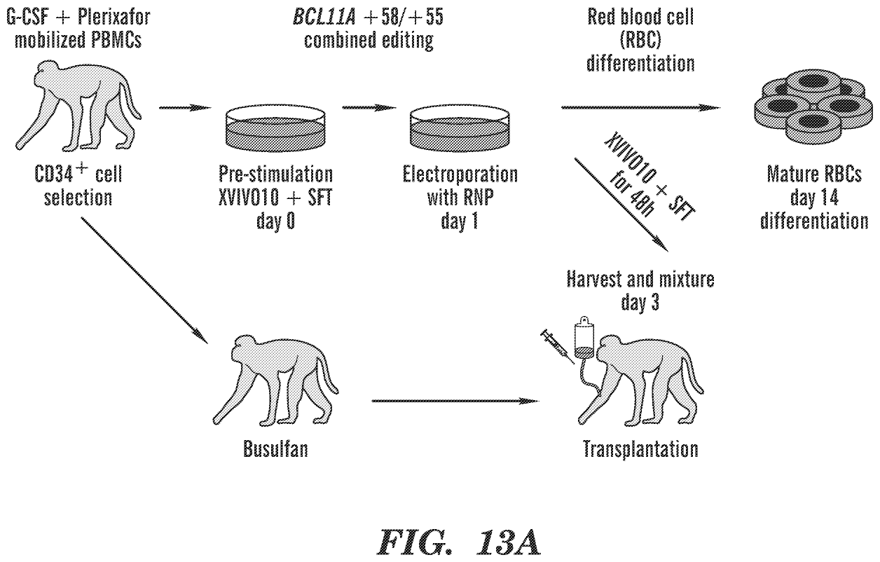

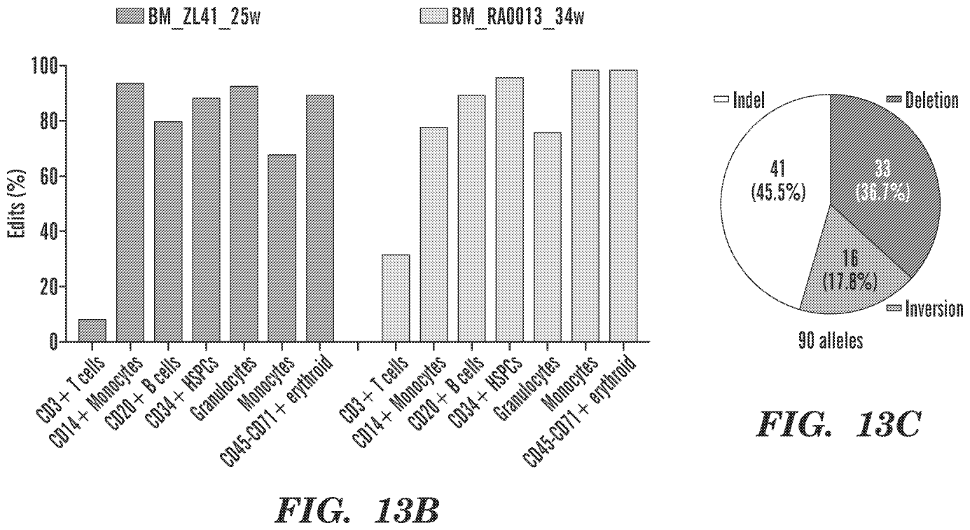

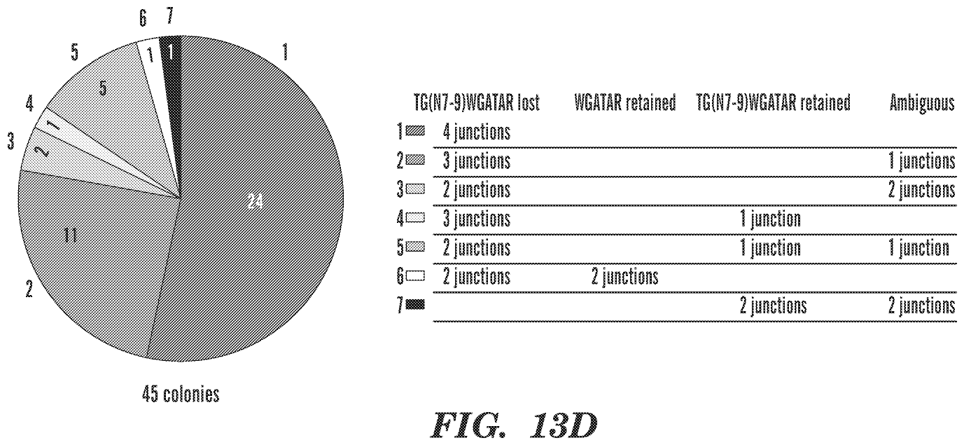

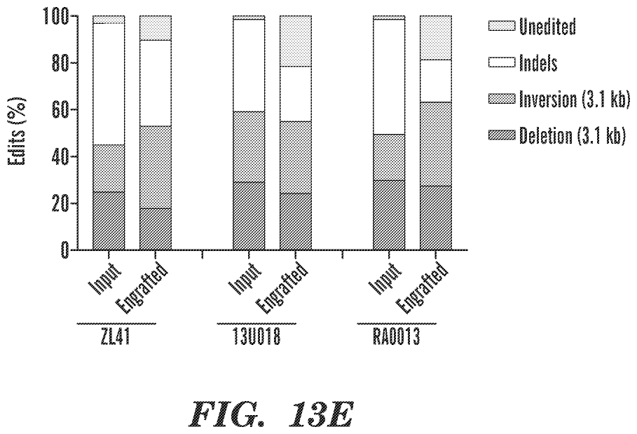

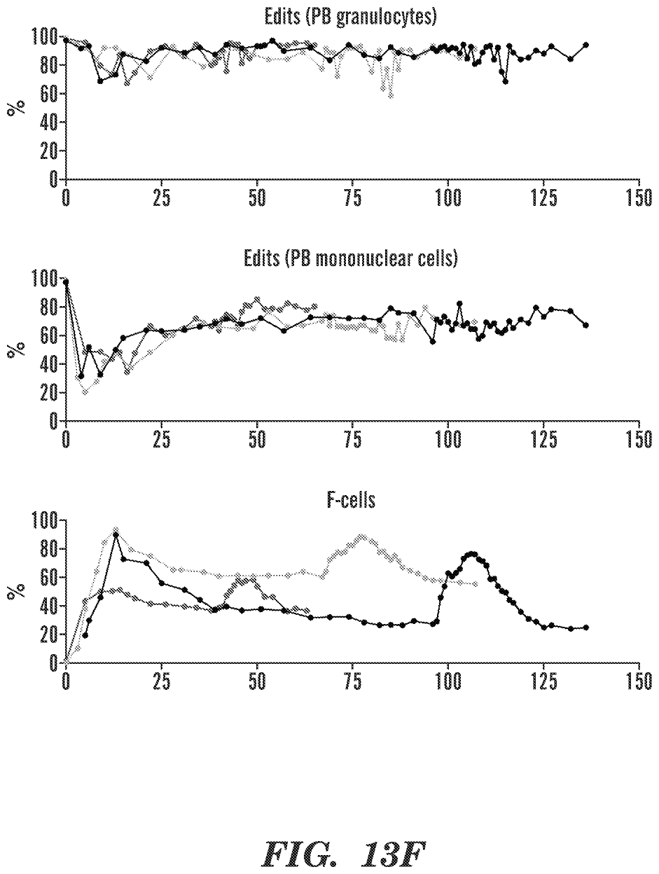

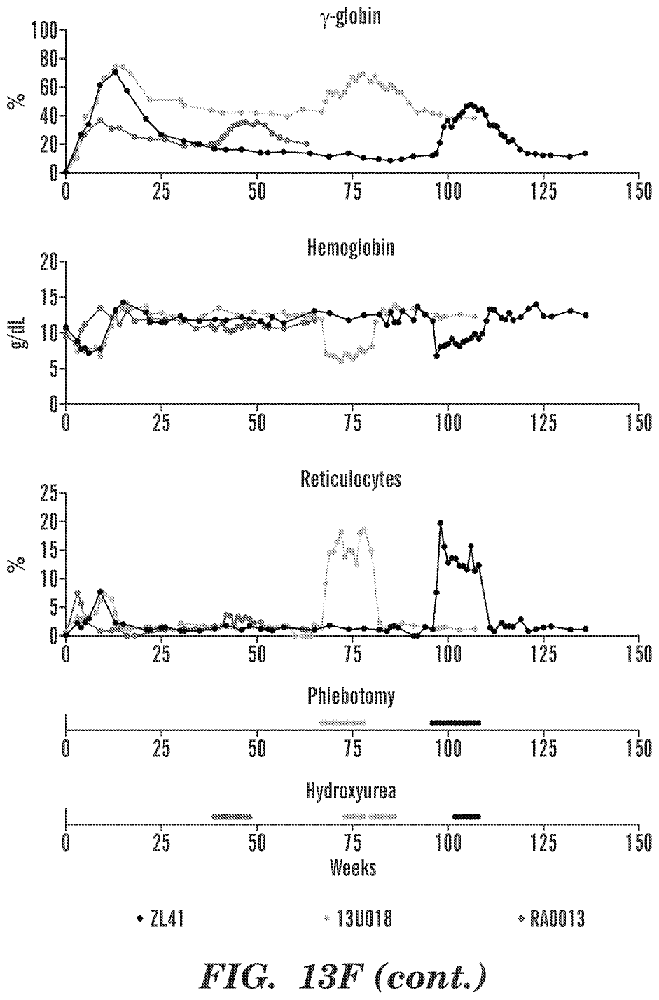

Targeting the BCL11A erythroid enhancer by gene editing is a promising approach to fetal hemoglobin induction for beta-hemoglobinopathies. HbF levels vary widely among individuals, suggesting potential heterogeneity in HbF responses after therapeutic intervention. Data provided herein (FIGS. 1A-13F) assessed if maximizing both gene edit frequency and HbF induction potential could promote consistently favorable clinical outcomes. CRISPR-Cas9 endonuclease editing of the BCL11A +58 enhancer with alternative gene modification approaches was assessed, including +55 erythroid enhancer editing alone or in combination with the +58 enhancer, as well as editing targeting the HBG1/2 promoter−115 BCL11A binding site and transduction by an shRNA knocking down the BCL11A transcript in erythroid precursors. It was found that combined targeting of the BCL11A +58 and +55 enhancers with 3×NLS-SpCas9 and two sgRNAs resulted in the most potent HbF induction (52.4%±6.3%) of tested approaches (BCL11A +58 editing alone, 29.1%±3.9%; BCL11A +55 editing alone, 34.8±5.1%; HBG1/2 promoter editing, 34.1%±5.4%; shmiR-BCL11A, 32.2%±4.4%; mock, 7.6%±3.4%).

Based on assays in bulk and single cell derived erythroid cultures and xenografted immunodeficient mice, it was found that disruption of core half E-box/GATA motifs at both the +58 and +55 enhancers was associated with greatest HbF induction, whether by small indels, interstitial 3.1 kb deletion, or 3.1 kb inversion. Rare gene edited clones with alleles that only partially disrupted these motifs were associated with intermediate HbF induction phenotypes. Combined editing of BCL11A +58 and +55 enhancers was compatible with HSC self-renewal in primary and secondary xenotransplant, with intact lymphoid, myeloid and erythroid repopulation. Gene-edited cell product manufacturing process development and developed conditions were conducted using a MaxCyte electroporation instrument achieving mean 97.3±1.8% gene edits and 88.9%±6.4% viability 24 hours after electroporation in 3 engineering runs at clinical scale. Similar results were obtained at small-scale with plerixafor-mobilized HSPCs from sickle cell disease (SCD) donors or G-CSF mobilized PBMCs from transfusion-dependent beta-thalassemia (TDT) donors, including 94.2%±4.4%, 99.5%±0.3% and 91.8%±6.3% of gene edits in engrafting cells from NBSGW 16 week mouse bone marrow of healthy, SCD and TDT donors respectively. Off-target analyses by pooled amplicon sequencing of 601 candidate off-target sites for the +58 and +55 targeting sgRNAs, nominated by a range of computational (CRISPRme) and experimental (GUIDE-seq and ONE-seq) methods, did not identify reference genome off-target edits at a sensitivity of 0.1% allele frequency.

+58/+55 enhancer combined targeting was evaluated in nonhuman primates by performing ribonucleoprotein (RNP) electroporation in rhesus macaque mobilized peripheral blood CD34+ HSPCs with autologous re-infusion following busulfan myeloablation. Highly efficient gene edit frequency (85.2%, 88.8% and 84.9%) and durable HbF induction (26.4%, 57.5%, and 45.9% F-cells and 12.7%, 41.9%, and 28% gamma-globin) was observed in the peripheral blood in 3 animals at most recent recorded time point post infusion (127, 78, and 54 weeks respectively). Single colony analyses and bulk ddPCR and unidirectional sequencing demonstrated that the long-term engrafting cells displayed a similar distribution of indels, 3.1 kb deletions, and 3.1 kb inversions as the input cell products. Erythroid stress due to hydroxyurea treatment, with or without phlebotomy, was associated with substantially augmented HbF responses (to 75.9%, 88.2%, and 57.8% F-cells and 47.9%, 68%, and 35.7% gamma-globin). No hematologic or other toxicities attributable to gene editing were observed.

Together these results indicate that combined BCL11A +58 and +55 erythroid enhancer editing produces highly efficient on-target allelic disruption, erythroid-specific BCL11A downregulation, heightened HbF induction capacity compared to alternative approaches, preserved long-term multilineage engraftment potential by both human xenotransplant and rhesus autotransplant assays, and absence of evident genotoxicity, under clinically relevant SpCas9 RNP electroporation conditions.

TABLE 3
sgRNA Target Sequences

| | | | | | | Chr2 | | |
|---|---|---|---|---|---|---|---|---|
| | | SEQ | | Target- | Coordinate | Genomic | | |
| | sgRNA | ID | | ed | Relative | Coordinate | Enrichment | Dropout |
| Identifer | Sequence | NO: | PAM | Site | to TSS | (hg19) | Score | Score |
| BCL_00108_ | TCTGAGGAGC | 36 | NGG | DHS_55 | 54701 | 60725932 | 0.3065268 | −0.64986 |
| H_D55 | TAGAGACTTG | | | | | | | |
| BCL_00096_ | AGCAAATAGG | 37 | NGG | DHS_55 | 54874 | 60725759 | 0.35208854 | −0.23956 |
| H_D55 | CTTAGTGTGC | | | | | | | |
| BCL_01427_ | GGCTAAATAA | 38 | NGG | DHS_55 | 54944 | 60725689 | 0.36697304 | −0.27163 |
| H_D55 | TGAATGTCCC | | RC | | | | | |
| BCL_00093_ | TCCCTTCCTA | 39 | NGG | DHS_55 | 54950 | 60725683 | 0.52834198 | −0.56164 |
| H_D55 | GAATTGGCCT | | | | | | | |
| BCL_00092_ | TTCCCTTCCT | 40 | NGG | DHS_55 | 54951 | 60725682 | 0.40353821 | −0.43691 |
| H_D55 | AGAATTGGCC | | | | | | | |
| BCL_01428_ | GAATGTCCCA | 41 | NGG | DHS_55 | 54955 | 60725678 | 0.4298807 | −0.54353 |
| H_D55 | GGCCAATTCT | | RC | | | | | |
| BCL_00091_ | CCCACTTCCC | 42 | NGG | DHS_55 | 54956 | 60725677 | 1.16779598 | −0.50425 |
| H_D55 | TTCCTAGAAT | | | | | | | |
| BCL_00090_ | CCTGGTACCA | 43 | NGG | DHS_55 | 54989 | 60725644 | 0.46505933 | −0.52917 |
| H_D55 | GGAAGGCAAT | | | | | | | |
| BCL_00089_ | TCCTGGTACC | 44 | NGG | DHS_55 | 54990 | 60725643 | 0.35594471 | −0.78622 |
| H_D55 | AGGAAGGCAA | | | | | | | |
| BCL_00088_ | GCATCATCCT | 45 | NGG | DHS_55 | 54996 | 60725637 | 0.43864112 | −0.37134 |
| H_D55 | GGTACCAGGA | | | | | | | |
| BCL_00087_ | CATTGCATCA | 46 | NGG | DHS_55 | 55000 | 60725633 | 0.43801718 | −0.22534 |
| H_D55 | TCCTGGTACC | | | | | | | |
| BCL_00086_ | CTCCAAGCAT | 47 | NGG | DHS_55 | 55007 | 60725626 | 0.63433419 | −0.27033 |
| H_D55 | TGCATCATCC | | | | | | | |
| BCL_01438_ | TACCAGGATG | 48 | NGG | DHS_55 | 55016 | 60725617 | 0.91292075 | −0.34122 |
| H_D55 | ATGCAATGCT | | RC | | | | | |
| BCL_00085_ | GGGTGTGCCC | 49 | NGG | DHS_55 | 55040 | 60725593 | 0.50114706 | −0.6263 |
| H_D55 | TGAGAAGGTG | | | | | | | |
| BCL_00084_ | AGGGTGTGCC | 50 | NGG | DHS_55 | 55041 | 60725592 | 0.31100243 | −0.36912 |
| H_D55 | CTGAGAAGGT | | | | | | | |
| BCL_00082_ | TCACAGGGTG | 51 | NGG | DHS_55 | 55045 | 60725588 | 0.41742767 | −1.08709 |
| H_D55 | TGCCCTGAGA | | | | | | | |
| BCL_01443_ | GGCACACCCT | 52 | NGG | DHS_55 | 55065 | 60725568 | 0.41807361 | 0.257924 |
| H_D55 | GTGATCTTGT | | RC | | | | | |
| BCL_00073_ | AGCACACAAG | 53 | NGG | DHS_55 | 55096 | 60725537 | 0.41986965 | −0.83722 |
| H_D55 | ATGCACACCC | | | | | | | |
| BCL_01448_ | TGTGCTTGGT | 54 | NGG | DHS_55 | 55124 | 60725509 | 1.34772811 | −0.49527 |
| H_D55 | CGGCACTGAT | | RC | | | | | |
| BCL_01449_ | GTGCTTGGTC | 55 | NGG | DHS_55 | 55125 | 60725508 | 1.13392025 | −0.61013 |
| H_D55 | GGCACTGATA | | RC | | | | | |
| BCL_01450_ | TGCTTGGTCG | 56 | NGG | DHS_55 | 55126 | 60725507 | 1.5783257 | −0.31949 |
| H_D55 | GCACTGATAG | | RC | | | | | |
| BCL_01454_ | GGGTCGCGGT | 57 | NGG | DHS_55 | 55146 | 60725487 | 0.35789318 | −0.55774 |
| H_D55 | AGGGAGTTGT | | RC | | | | | |
| BCL_00065_ | GCCAACAGTG | 58 | NGG | DHS_55 | 55235 | 60725398 | 0.48864454 | −0.54147 |
| H_D55 | ATAACCAGCA | | | | | | | |
| BCL_00064_ | TGCCAACAGT | 59 | NGG | DHS_55 | 55236 | 60725397 | 0.51080164 | −0.35814 |
| H_D55 | GATAACCAGC | | | | | | | |
| BCL_01461_ | GCCCTGCTGG | 60 | NGG | DHS_55 | 55245 | 60725388 | 0.59624098 | −0.51154 |
| H_D55 | TTATCACTGT | | RC | | | | | |
| BCL_00062_ | AGCAGCCCTG | 61 | NGG | DHS_55 | 55272 | 60725361 | 0.32514466 | −0.64013 |
| H_D55 | GGCACAGAAG | | | | | | | |
| BCL_00058_ | CCTCTATGTA | 62 | NGG | DHS_55 | 55311 | 60725322 | 0.32368336 | −0.4848 |
| H_D55 | GACGGGTGTG | | | | | | | |
| BCL_00057_ | GGAAGGGCCT | 63 | NGG | DHS_55 | 55318 | 60725315 | 0.45996809 | −0.44507 |
| H_D55 | CTATGTAGAC | | | | | | | |
| BCL_00051_ | GGAGGTGTGG | 64 | NGG | DHS_55 | 55356 | 60725277 | 0.31408916 | −0.16554 |
| H_D55 | AGGGGATAAC | | | | | | | |
| BCL_00031_ | CTGGCAGACC | 65 | NGG | DHS_55 | 55444 | 60725189 | 0.32158621 | −1.35414 |
| H_D55 | CTCAAGAGCA | | | | | | | |
| BCL_00027_ | CCCATGGAGG | 66 | NGG | DHS_55 | 55474 | 60725159 | 0.28225491 | −0.45625 |
| H_D55 | TGGGGAGATG | | | | | | | |
| BCL_01483_ | GTCATCCTCG | 67 | NGG | DHS_55 | 55559 | 60725074 | 0.43184473 | −0.40557 |
| H_D55 | GCCAATGAAG | | RC | | | | | |
| BCL_00012_ | AAGTGAGCCA | 68 | NGG | DHS_55 | 55585 | 60725048 | 0.35107033 | −0.01983 |
| H_D55 | GGTGATAGAA | | | | | | | |
| BCL_00008_ | TGAAACCAAG | 69 | NGG | DHS_55 | 55612 | 60725021 | 0.27412127 | −0.23029 |
| H_D55 | CTTCCTCTGC | | | | | | | |
| BCL_01495_ | AGGGAGAAAT | 70 | NGG | DHS_55 | 55700 | 60724933 | 0.26434414 | −0.49318 |
| H_D55 | GAGACAAAAG | | RC | | | | | |
| BCL_01497_ | AAGAGGCCAC | 71 | NGG | DHS_55 | 55717 | 60724916 | 0.43002762 | 0.456237 |
| H_D55 | TGAGTCCTTT | | RC | | | | | |
| BCL_01617_ | CTAACAGTTG | 72 | NGG | DHS_58 | 58232 | 60722401 | 2.04948208 | −0.71934 |
| H_D58 | CTTTTATCAC | | RC | | | | | |
| BCL_01618_ | TTGCTTTTAT | 73 | NGG | DHS_58 | 58239 | 60722394 | 0.85613918 | −0.81273 |
| H_D58 | CACAGGCTCC | | RC | | | | | |
| BCL_01619_ | TTTTATCACA | 74 | NGG | DHS_58 | 58243 | 60722390 | 1.66244771 | −0.31469 |
| H_D58 | GGCTCCAGGA | | RC | | | | | |
| BCL_01620_ | TTTATCACAG | 75 | NGG | DHS_58 | 58244 | 60722389 | 1.38026011 | −0.94808 |
| H_D58 | GCTCCAGGAA | | RC | | | | | |
| BCL_00187_ | ATCAGAGGCC | 76 | NGG | DHS_58 | 58246 | 60722387 | 2.12232899 | −0.74438 |
| H_D58 | AAACCCTTCC | | | | | | | |
| BCL_01621_ | CACAGGCTCC | 77 | NGG | DHS_58 | 58249 | 60722384 | 2.31905068 | −0.60048 |
| H_D58 | AGGAAGGGTT | | RC | | | | | |
| BCL_00186_ | CACGCCCCCA | 78 | NGG | DHS_58 | 58261 | 60722372 | 0.89714161 | −0.79647 |
| H_D58 | CCCTAATCAG | | | | | | | |
| BCL_01622_ | GAAGGGTTTG | 79 | NGG | DHS_58 | 58261 | 60722372 | 1.37845184 | −0.66954 |
| H_D58 | GCCTCTGATT | | RC | | | | | |
| BCL_01623_ | AAGGGTTTGG | 80 | NGG | DHS_58 | 58262 | 60722371 | 1.28521056 | −0.26686 |
| H_D58 | CCTCTGATTA | | RC | | | | | |
| BCL_01624_ | GGTTTGGCCT | 81 | NGG | DHS_58 | 58265 | 60722368 | 1.47218462 | −0.77128 |
| H_D58 | CTGATTAGGG | | RC | | | | | |
| BCL_01625_ | GTTTGGCCTC | 82 | NGG | DHS_58 | 58266 | 60722367 | 0.37182118 | −0.94511 |
| H_D58 | TGATTAGGGT | | RC | | | | | |
| BCL_01626_ | TTTGGCCTCT | 83 | NGG | DHS_58 | 58267 | 60722366 | 1.33557005 | −0.27239 |
| H_D58 | GATTAGGGTG | | RC | | | | | |
| BCL_01627_ | TTGGCCTCTG | 84 | NGG | DHS_58 | 58268 | 60722365 | 0.30537167 | −0.2564 |
| H_D58 | ATTAGGGTGG | | RC | | | | | |
| BCL_01629_ | TCTGATTAGG | 85 | NGG | DHS_58 | 58274 | 60722359 | 1.10417515 | 0.18067 |
| H_D58 | GTGGGGGCGT | | RC | | | | | |
| BCL_01631_ | ATTAGGGTGG | 86 | NGG | DHS_58 | 58278 | 60722355 | 0.40981324 | −0.16153 |
| H_D58 | GGGCGTGGGT | | RC | | | | | |
| BCL_01634_ | TGGGTGGGGT | 87 | NGG | DHS_58 | 58293 | 60722340 | 0.41467523 | −1.07834 |
| H_D58 | AGAAGAGGAC | | RC | | | | | |
| BCL_00185_ | GCAAACGGCC | 88 | NGG | DHS_58 | 58309 | 60722324 | 0.3196407 | −0.51601 |
| H_D58 | ACCGATGGAG | | | | | | | |
| BCL_00184_ | CCTGGGCAAA | 89 | NGG | DHS_58 | 58314 | 60722319 | 0.31547607 | −0.54143 |
| H_D58 | CGGCCACCGA | | | | | | | |
| BCL_00183_ | AAGAGGCCCC | 90 | NGG | DHS_58 | 58324 | 60722309 | 0.78527241 | −0.59129 |
| H_D58 | CCTGGGCAAA | | | | | | | |
| BCL_01637_ | CCATCGGTGG | 91 | NGG | DHS_58 | 58325 | 60722308 | 0.66904064 | −0.50156 |
| H_D58 | CCGTTTGCCC | | RC | | | | | |
| BCL_01638_ | CATCGGTGGC | 92 | NGG | DHS_58 | 58326 | 60722307 | 0.63502753 | −0.59285 |
| H_D58 | CGTTTGCCCA | | RC | | | | | |
| BCL_01639_ | ATCGGTGGCC | 93 | NGG | DHS_58 | 58327 | 60722306 | 0.82185918 | −0.89805 |
| H_D58 | GTTTGCCCAG | | RC | | | | | |
| BCL_01640_ | TCGGTGGCCG | 94 | NGG | DHS_58 | 58328 | 60722305 | 0.36580154 | −1.01297 |
| H_D58 | TTTGCCCAGG | | RC | | | | | |
| BCL_01641_ | CGGTGGCCGT | 95 | NGG | DHS_58 | 58329 | 60722304 | 0.28196886 | −0.46328 |
| H_D58 | TTGCCCAGGG | | RC | | | | | |
| BCL_00182_ | CTTCCGAAAG | 96 | NGG | DHS_58 | 58331 | 60722302 | 0.29420004 | 0.023956 |
| H_D58 | AGGCCCCCCT | | | | | | | |
| BCL_00181_ | CCTTCCGAAA | 97 | NGG | DHS_58 | 58332 | 60722301 | 0.33994629 | 0.262073 |
| H_D58 | GAGGCCCCCC | | | | | | | |
| BCL_00160_ | TCAGGGGGAG | 98 | NGG | DHS_58 | 58575 | 60722058 | 0.32935479 | −0.31801 |
| H_D58 | GCAAGTCAGT | | | | | | | |
| BCL_00154_ | AGGGAAAAGG | 99 | NGG | DHS_58 | 58612 | 60722021 | 0.4446489 | −0.39917 |
| H_D58 | GAGAGGAAAA | | | | | | | |
| BCL_01665_ | TGTAACTAAT | 100 | NGG | DHS_58 | 58706 | 60721927 | 0.44183247 | −0.65165 |
| H_D58 | AAATACCAGG | | RC | | | | | |
| BCL_01669_ | CCAGCTGAAG | 101 | NGG | DHS_58 | 58870 | 60721763 | 0.31959971 | −0.00075 |
| H_D58 | AAAGAACATT | | RC | | | | | |
| BCL_00135_ | CCATCTCCCT | 102 | NGG | DHS_58 | 58958 | 60721675 | 0.29845544 | −0.04502 |
| H_D58 | AATCTCCAAT | | | | | | | |
| BCL_00131_ | TGGGGAGAGA | 103 | NGG | DHS_58 | 59030 | 60721603 | 0.26979883 | −0.3654 |
| H_D58 | AGAGTGGAAA | | | | | | | |
| BCL_00130_ | GGAGTATGGG | 104 | NGG | DHS_58 | 59036 | 60721597 | 0.37521645 | −2.21246 |
| H_D58 | GAGAGAAGAG | | | | | | | |
| BCL_01684_ | ACAACCTCCT | 105 | NGG | DHS_58 | 59129 | 60721504 | 0.49451625 | 0.36739 |
| H_D58 | TGTTTACAGA | | RC | | | | | |
| BCL_01788_ | GAGATTTACT | 106 | NGG | DHS_62 | 61848 | 60718785 | 1.29003182 | −5.46287 |
| H_D62 | CTTGTTGCCC | | RC | | | | | |
| BCL_01790_ | TTGCCCGGGC | 107 | NGG | DHS_62 | 61862 | 60718771 | 0.46730546 | −8.12292 |
| H_D62 | TGGAATGCAA | | RC | | | | | |
| BCL_00245_ | GGAGATCGCT | 108 | NGG | DHS_62 | 61901 | 60718732 | 0.47622708 | −5.06663 |
| H_D62 | TGAACCTGGG | | | | | | | |
| BCL_00241_ | CTCAGCTACT | 109 | NGG | DHS_62 | 61926 | 60718707 | 0.6113192 | −9.05154 |
| H_D62 | CGGGAGGCTG | | | | | | | |
| BCL_00240_ | TGTAATCTCA | 110 | NGG | DHS_62 | 61932 | 60718701 | 0.79003182 | −8.69099 |
| H_D62 | GCTACTCGGG | | | | | | | |
| BCL_00239_ | GCCTGTAATC | 111 | NGG | DHS_62 | 61935 | 60718698 | 1.91594174 | −6.03102 |
| H_D62 | TCAGCTACTC | | | | | | | |
| BCL_00238_ | TGCCTGTAAT | 112 | NGG | DHS_62 | 61936 | 60718697 | 0.6113192 | −8.92274 |
| H_D62 | CTCAGCTACT | | | | | | | |
| BCL_01794_ | CAGGCATGTA | 113 | NGG | DHS_62 | 61964 | 60718669 | 0.28012743 | −1.01079 |
| H_D62 | TTACCATGCC | | RC | | | | | |
| BCL_00233_ | CAGGAGGATC | 114 | NGG | DHS_62 | 62037 | 60718596 | 0.6113192 | −9.20231 |
| H_D62 | ACCTGAGGTC | | | | | | | |
| BCL_01799_ | CTCAGGTGAT | 115 | NGG | DHS_62 | 62054 | 60718579 | 0.91082485 | −9.47845 |
| H_D62 | CCTCCTGCCC | | RC | | | | | |
| BCL_00229_ | CCCAGCACTT | 116 | NGG | DHS_62 | 62060 | 60718573 | 0.6113192 | −8.71688 |
| H_D62 | TGGGAGGCCG | | | | | | | |
| BCL_00228_ | TCCCAGCACT | 117 | NGG | DHS_62 | 62061 | 60718572 | 0.76104471 | −5.65759 |
| H_D62 | TTGGGAGGCC | | | | | | | |
| BCL_00227_ | ATCCCAGCAC | 118 | NGG | DHS_62 | 62062 | 60718571 | 0.79003182 | −8.09896 |
| H_D62 | TTTGGGAGGC | | | | | | | |
| BCL_00225_ | ACCTGTAATC | 119 | NGG | DHS_62 | 62069 | 60718564 | 0.33277348 | −8.82052 |
| H_D62 | CCAGCACTTT | | | | | | | |
| BCL_01800_ | GCCCCGGCCT | 120 | NGG | DHS_62 | 62070 | 60718563 | 0.6113192 | −7.64956 |
| H_D62 | CCCAAAGTGC | | RC | | | | | |
| BCL_01801_ | CCCCGGCCTC | 121 | NGG | DHS_62 | 62071 | 60718562 | 0.6113192 | −8.0566 |
| H_D62 | CCAAAGTGCT | | RC | | | | | |
| BCL_01825_ | ATTTGCTCTT | 122 | NGG | DHS_62 | 62469 | 60718164 | 0.28180883 | −0.39453 |
| H_D62 | CTCCAGGGTG | | RC | | | | | |
| BCL_00210_ | TAAACAGCCA | 123 | NGG | DHS_62 | 62470 | 60718163 | 0.70263344 | −0.87051 |
| H_D62 | CCCCACACCC | | | | | | | |
| BCL_01826_ | TTTGCTCTTC | 124 | NGG | DHS_62 | 62470 | 60718163 | 0.40028858 | −0.33863 |
| H_D62 | TCCAGGGTGT | | RC | | | | | |
| BCL_01828_ | CTCTTCTCCA | 125 | NGG | DHS_62 | 62474 | 60718159 | 0.34846068 | −0.39104 |
| H_D62 | GGGTGTGGGG | | RC | | | | | |
| BCL_01829_ | TGTGGGGTGG | 126 | NGG | DHS_62 | 62487 | 60718146 | 0.49598477 | −0.14693 |
| H_D62 | CTGTTTAAAG | | RC | | | | | |
| BCL_01831_ | GGGTGGCTGT | 127 | NGG | DHS_62 | 62491 | 60718142 | 0.41044562 | −0.14856 |
| H_D62 | TTAAAGAGGG | | RC | | | | | |
| BCL_01833_ | AGTTCAAGTA | 128 | NGG | DHS_62 | 62580 | 60718053 | 0.61158376 | 0.228869 |
| H_D62 | GATATCAGAA | | RC | | | | | |
| BCL_01834_ | TATCAGAAGG | 129 | NGG | DHS_62 | 62592 | 60718041 | 0.40286685 | 0.023271 |
| H_D62 | GAACTGTTTG | | RC | | | | | |
| BCL_02015_ | AAGAATGGCT | 130 | NGG | exon2 | 7218 | 60773415 | 1.06436679 | −1.34908 |
| H_exon2 | TCAAGAGGCT | | RC | | | | | |
| BCL_02014_ | TCTGTAAGAA | 131 | NGG | exon2 | 7223 | 60773410 | 0.99011778 | −0.7711 |
| H_exon2 | TGGCTTCAAG | | RC | | | | | |
| BCL_00248_ | ACAGATGATG | 132 | NGG | exon2 | 7224 | 60773409 | 1.60874074 | −2.53181 |
| H_exon2 | AACCAGACCA | | | | | | | |
| BCL_00249_ | TGAACCAGAC | 133 | NGG | exon2 | 7232 | 60773401 | 1.1752178 | −0.82211 |
| H_exon2 | CACGGCCCGT | | | | | | | |
| BCL_00250_ | GAACCAGACC | 134 | NGG | exon2 | 7233 | 60773400 | 1.58125311 | −0.68474 |
| H_exon2 | ACGGCCCGTT | | | | | | | |
| BCL_00251_ | GGCCCGTTGG | 135 | NGG | exon2 | 7245 | 60773388 | 1.91082485 | −1.23576 |
| H_exon2 | GAGCTCCAGA | | | | | | | |
| BCL_00252_ | GCCCGTTGGG | 136 | NGG | exon2 | 7246 | 60773387 | 0.54529072 | 0.092119 |
| H_exon2 | AGCTCCAGAA | | | | | | | |
| BCL_00253_ | CCCGTTGGGA | 137 | NGG | exon2 | 7247 | 60773386 | 1.20485173 | −1.96839 |
| H_exon2 | GCTCCAGAAG | | | | | | | |
| BCL_02011_ | CTGGAGCTCC | 138 | NGG | exon2 | 7258 | 60773375 | 0.6044195 | 0.791184 |
| H_exon2 | CAACGGGCCG | | RC | | | | | |
| BCL_02010_ | CCCCTTCTGG | 139 | NGG | exon2 | 7264 | 60773369 | 0.50032578 | −0.14628 |
| H_exon2 | AGCTCCCAAC | | RC | | | | | |
| BCL_02009_ | TCCCCTTCTG | 140 | NGG | exon2 | 7265 | 60773368 | 2.10774428 | −1.69298 |
| H_exon2 | GAGCTCCCAA | | RC | | | | | |
| BCL_00254_ | GATCATGACC | 141 | NGG | exon2 | 7269 | 60773364 | 2.19780485 | −2.25564 |
| H_exon2 | TCCTCACCTG | | | | | | | |
| BCL_00255_ | ATCATGACCT | 142 | NGG | exon2 | 7270 | 60773363 | 1.70330708 | −2.49715 |
| H_exon2 | CCTCACCTGT | | | | | | | |
| BCL_02008_ | AGGAGGTCAT | 143 | NGG | exon2 | 7277 | 60773356 | 0.34947658 | −0.44825 |
| H_exon2 | GATCCCCTTC | | RC | | | | | |
| BCL_02007_ | GGCACTGCCC | 144 | NGG | exon2 | 7294 | 60773339 | 3.35094127 | −1.66199 |
| H_exon2 | ACAGGTGAGG | | RC | | | | | |
| BCL_00256_ | GTGCCAGATG | 145 | NGG | exon2 | 7295 | 60773338 | 1.89017832 | −1.76407 |
| H_exon2 | AACTTCCCAT | | | | | | | |
| BCL_00257_ | TGCCAGATGA | 146 | NGG | exon2 | 7296 | 60773337 | 1.94508027 | −1.9609 |
| H_exon2 | ACTTCCCATT | | | | | | | |
| BCL_00258_ | GCCAGATGAA | 147 | NGG | exon2 | 7297 | 60773336 | 1.59275545 | −1.89857 |
| H_exon2 | CTTCCCATTG | | | | | | | |
| BCL_02006_ | TCTGGCACTG | 148 | NGG | exon2 | 7297 | 60773336 | 1.48917633 | −2.02947 |
| H_exon2 | CCCACAGGTG | | RC | | | | | |
| BCL_00259_ | CCAGATGAAC | 149 | NGG | exon2 | 7298 | 60773335 | 3.26617426 | −3.32127 |
| H_exon2 | TTCCCATTGG | | | | | | | |
| BCL_02005_ | GTTCATCTGG | 150 | NGG | exon2 | 7302 | 60773331 | 3.20226887 | −1.83694 |
| H_exon2 | CACTGCCCAC | | RC | | | | | |
| BCL_02004_ | CCCCCAATGG | 151 | NGG | exon2 | 7315 | 60773318 | 0.46854155 | −0.11887 |
| H_exon2 | GAAGTTCATC | | RC | | | | | |
| BCL_02003_ | AAATAAGAAT | 152 | NGG | exon2 | 7327 | 60773306 | 1.08475851 | −0.09695 |
| H_exon2 | GTCCCCCAAT | | RC | | | | | |
| BCL_02002_ | AAAATAAGAA | 153 | NGG | exon2 | 7328 | 60773305 | 0.50500271 | −0.4259 |
| H_exon2 | TGTCCCCCAA | | RC | | | | | |
| BCL_00261_ | CACAAACGGA | 154 | NGG | exon2 | 7341 | 60773292 | 3.32908014 | −2.54324 |
| H_exon2 | AACAATGCAA | | | | | | | |
| BCL_00262_ | CCTCTGCTTA | 155 | NGG | exon2 | 7367 | 60773266 | 1.00055405 | −1.35239 |
| H_exon2 | GAAAAAGCTG | | | | | | | |
| BCL_02001_ | CCACAGCTTT | 156 | NGG | exon2 | 7384 | 60773249 | 0.49127532 | −0.24954 |
| H_exon2 | TTCTAAGCAG | | RC | | | | | |
| BCL_02000_ | TCGATTGGTG | 157 | NGG | exon2 | 7412 | 60773221 | 0.46242001 | −1.36477 |
| H_exon2 | AAGGGGAAGG | | RC | | | | | |
| BCL_01999_ | ATCTCGATTG | 158 | NGG | exon2 | 7415 | 60773218 | 0.62036667 | −0.76015 |
| H_exon2 | GTGAAGGGGA | | RC | | | | | |
| BCL_01998_ | TTTCATCTCG | 159 | NGG | exon2 | 7419 | 60773214 | 0.34887409 | −0.14262 |
| H_exon2 | ATTGGTGAAG | | RC | | | | | |
| BCL_00263_ | GAAAAAAGCA | 160 | NGG | exon2 | 7421 | 60773212 | 0.6213377 | −2.11505 |
| H_exon2 | TCCAATCCCG | | | | | | | |
| BCL_00264_ | AAAAGCATCC | 161 | NGG | exon2 | 7424 | 60773209 | 0.55781702 | −1.37569 |
| H_exon2 | AATCCCGTGG | | | | | | | |
| BCL_00265_ | GCATCCAATC | 162 | NGG | exon2 | 7428 | 60773205 | 1.290845 | −0.88953 |
| H_exon2 | CCGTGGAGGT | | | | | | | |
| BCL_00266_ | TCCCGTGGAG | 163 | NGG | exon2 | 7436 | 60773197 | 0.58892468 | −0.18023 |
| H_exon2 | GTTGGCATCC | | | | | | | |
| BCL_00267_ | TGGCATCCAG | 164 | NGG | exon2 | 7448 | 60773185 | 2.04934363 | −2.00635 |
| H_exon2 | GTCACGCCAG | | | | | | | |
| BCL_01994_ | GATGCCAACC | 165 | NGG | exon2 | 7449 | 60773184 | 1.10977009 | −0.99042 |
| H_exon2 | TCCACGGGAT | | RC | | | | | |
| BCL_01993_ | ACCTGGATGC | 166 | NGG | exon2 | 7454 | 60773179 | 1.97417272 | −1.73599 |
| H_exon2 | CAACCTCCAC | | RC | | | | | |
| BCL_01992_ | GACCTGGATG | 167 | NGG | exon2 | 7455 | 60773178 | 1.23389832 | −0.6955 |
| H_exon2 | CCAACCTCCA | | RC | | | | | |
| BCL_01991_ | CGTCATCCTC | 168 | NGG | exon2 | 7471 | 60773162 | 0.85232011 | −0.71662 |
| H_exon2 | TGGCGTGACC | | RC | | | | | |
| BCL_01990_ | GATAAACAAT | 169 | NGG | exon2 | 7481 | 60773152 | 0.84221705 | −0.61283 |
| H_exon2 | CGTCATCCTC | | RC | | | | | |
| BCL_01989_ | CTGCTATGTG | 170 | NGG | exon2 | 7525 | 60773108 | 0.62008756 | 0.033203 |
| H_exon2 | TTCCTGTTTG | | RC | | | | | |

TABLE 4
sgRNA Target Sequences

| sgRNA Target Gene or Region | Species | Sequence | SEQ ID NO: |
|---|---|---|---|
| Composite Enhancer 5′ Target 1 | Human | TGGAAAGGAGAACGGCCCGG | 171 |
| Composite Enhancer 5′ Target 2 | Human | TGAACACCCTCGTTAAAGGC | 172 |
| Composite Enhancer 5′ Target 3 | Human | AACACTAGCCCACATGCCAA | 173 |
| Composite Enhancer 3′ Target 1 | Human | GCCCACAGAGGCACGGTTAA | 174 |
| Composite Enhancer 3′ Target 2 | Human | AGGCACGGTTAATGGTGGCG | 175 |
| Composite Enhancer 3′ Target 3 | Human | CACAGGAAGCCATGGTCCTT | 176 |
| +55 5′ Target 1 | Human | GCACTGACGTAGGTAGTGAC | 177 |
| +55 5′ Target 2 | Human | ATAGGATATGGCACTGACGT | 178 |
| +55 3′ Target 1 | Human | CATTATCTTCTCTGGTCTCG | 179 |
| +55 3′ Target 2 | Human | ATACTGGGGAACACATTGTA | 180 |
| +58 5′ Target 1 | Human | TGAGCACATTCTTACGCCTA | 181 |
| +58 5′ Target 2 | Human | CTAGGCGTAAGAATGTGCTC | 182 |
| +58 3′ Target 1 | Human | GAACCCCCTATAAACTAGTC | 183 |
| +58 3′ Target 2 | Human | GGCAAACCAGACTAGTTTAT | 184 |
| +62 5′ Target 1 | Human | CAGGGGAGAACTCGGCATGA | 185 |
| +62 5′ Target 2 | Human | GATGGAGTTGGTTGACCGTA | 186 |
| +62 3′ Target 1 | Human | GGTAGGACCCAACACTACGC | 187 |
| +62 3′ Target 2 | Human | ATGCCTAGGGTGTTTTGACG | 188 |
| BCL11A Exon 2 Target 2 | Human | TGAACCAGACCACGGCCCGT | 189 |
| BCL11A Exon 2 Target 3 | Human | GCATCCAATCCCGTGGAGGT | 190 |
| +55 5′ Target | Mouse | CACTGGCTTCCTGTTCTTGT | 191 |
| +55 3′ Target | Mouse | AAGGTTTTCAAGGCAAATAA | 192 |
| +58 5′ Target | Mouse | GTAATGGAGCCCGCATGCTG | 193 |
| +58 3′ Target | Mouse | GCCAGTGTACAGGCAAGTAC | 194 |
| +62 5′ Target | Mouse | TCGCTGCCTTCAGTTCTGCT | 195 |
| +62 3′ Target | Mouse | TTATGGAACTCAGGAACTGC | 196 |
| Bcl11a Exon 2 Target | Mouse | GATGCCTTTTTCATCTCGAT | 197 |
| +62 Heat Map Target 1 | Mouse | ATTCCTTGAGTGTCATATAT | 198 |
| +62 Heat Map Target 2 | Mouse | TCTGGAATCACTATGTATAT | 199 |

1. A vector comprising at least two guide RNAs (gRNAs) that targets and hybridizes to a target sequence on a DNA molecule, wherein each of the at least two gRNAs hybridizes at least two genomic DNA locations selected from human chromosome 2 at location 60725424 to 60725688 according to GRCh37/hg19 human genome reference build (+55 functional region); human chromosome 2 at location 60722238 to 60722466 GRCh37/hg19 human genome reference build (+58 functional region); or human chromosome 2 at location 60718042 to 60718186 according to GRCh37/hg19 human genome reference build (+62 functional region).

2. The vector of claim 1, wherein the gRNA has a selected from the group consisting of #1617: CUAACAGUUGCUUUUAUCAC (SEQ ID NO: 1),
#1618: UUGCUUUUAUCACAGGCUCC (SEQ ID NO: 2),
#1449: GUGCUUGGUCGGCACUGAUA (SEQ ID NO: 3), and
#1450: UGCUUGGUCGGCACUGAUAG (SEQ ID NO: 4).

3. The vector of claim 1, further comprising a DNA endonuclease enzyme.

4. A ribonucleoprotein (RNP) complex comprising a DNA endonuclease enzyme and at least two guide RNAs (gRNAs) that targets and hybridizes to a target sequence on a DNA molecule, wherein each of the at least two gRNAs hybridizes at least two genomic DNA locations selected from human chromosome 2 at location 60725424 to 60725688 according to GRCh37/hg19 human genome reference build (+55 functional region); human chromosome 2 at location 60722238 to 60722466 GRCh37/hg19 human genome reference build (+58 functional region); or human chromosome 2 at location 60718042 to 60718186 according to GRCh37/hg19 human genome reference build (+62 functional region).

5. The RNP of claim 4, wherein the at least two gRNAs hybridize to the same genomic DNA location, or wherein the at least two gRNAs hybridize to different genomic DNA locations.

6. (canceled)

7. The RNP of claim 4, wherein the RNP has one gRNA that hybridizes to the +55 functional region and one gRNA that hybridizes to the +58 functional region, or
wherein the RNP has one gRNA that hybridizes to the +55 functional region and one 9RNA that hybridizes to the +62 functional region 1, or
wherein the RNP has one gRNA that hybridizes to the +58 functional region and one 9RNA that hybridizes to the +62 functional region.

8.-10. (canceled)

11. The RNP of claim 4, wherein the DNA endonuclease comprises at least one of:
at least one nuclear localization signal (NLS) sequence fused at or near its amino terminus,
at least one NLS sequence fused at or near its carboxy terminus, and
one NLS sequence fused at or near its amino terminus, and two nuclear localization signal sequences fused at or near its carboxy terminus.

12. (canceled)

13. (canceled)

14. The RNP of claim 11, wherein the NLS sequence is selected from the group consisting of: SV40 large T-antigen, nucleoplasmin, c-Myc, c-Myc-like, and hRNPA1.

15. The RNP of claim 11, wherein the at least one nuclear localization signal sequences are identical or different.

16. (canceled)

17. The RNP of claim 4, wherein the DNA endonuclease has a c-Myc-like nuclear localization signal sequence fused to its amino terminus and an SV40 nuclear localization signal sequence and nucleplasmin bipartate nuclear localization signal sequence fused to its carboxyl terminus.

18. The RNP of claim 4, wherein the DNA endonuclease is selected from the group consisting of a Zinc Finger Nuclease (ZFN), a Transcription Activator-Like Effector Nuclease (TALENs), and a CRISPR-associated protein (Cas) nuclease.

19. The RNP of claim 18, wherein the CRISPR enzyme is a type II CRISPR system enzyme or a Cas protein.

20. (canceled)

21. The RNP of claim 19, wherein the Cas protein is selected from the group consisting of: Cpf1, C2c1, C2c3, Cas12a, Cas12b, Cas12c, Cas12d, Cas12e, Cas13a, Cas13b, and Cas13c. Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c.

22. (canceled)

23. A vector comprising the RNP of claim 4.

24. A composition comprising the vector of claim 1.

25. (canceled)

26. A method for producing a progenitor cell having decreased BCL11A mRNA or protein expression, the method comprising contacting an isolated progenitor cell with a vector of claim 1-3.

27. (canceled)

28. The method of claim 26, wherein the progenitor cell or is a hematopoietic progenitor cell or an induced pluripotent stem cell.

29. The method of claim 28, wherein the hematopoietic progenitor is a cell of the erythroid lineage.

30. (canceled)

31. The method of claim 28, wherein the contacting is ex vivo or in vitro contacting.

32. (canceled)

33. A composition comprising progenitor cells obtained by the method of claim 26.

34.-42. (canceled)


SEQUENCE LISTING (231 sequences)

SEQ ID NO: 1 - RNA, 20 nt, synthetic construct
  source 1..20: mol_type = other RNA
ctaacagttgcttttatcac

SEQ ID NO: 2 - RNA, 20 nt, synthetic construct
  source 1..20: mol_type = other RNA
ttgcttttatcacaggctcc

SEQ ID NO: 3 - RNA, 20 nt, synthetic construct
  source 1..20: mol_type = other RNA
gtgcttggtcggcactgata

SEQ ID NO: 4 - RNA, 20 nt, synthetic construct
  source 1..20: mol_type = other RNA
tgcttggtcggcactgatag

SEQ ID NO: 5 - protein, 4 aa, synthetic construct
GGSG

SEQ ID NO: 6 - protein, 12 aa, synthetic construct
GGSGGGSGGGSG

SEQ ID NO: 7 - protein, 20 aa, synthetic construct
GGSGGGSGGGSGGGSGGGSG

SEQ ID NO: 8 - protein, 15 aa, synthetic construct
TGGGPGGGAAAGSGS

SEQ ID NO: 9 - protein, 32 aa, synthetic construct
SGGSSGGSSGSETPGTSESATPESSGGSSGGS

SEQ ID NO: 10 - DNA, 12 bp, synthetic construct
  source 1..12: mol_type = other DNA
ggcggtagcggc

SEQ ID NO: 11 - DNA, 36 bp, synthetic construct
  source 1..36: mol_type = other DNA
ggcggtagcggcggaggcagcggtggcggcagcggc

SEQ ID NO: 12 - DNA, 60 bp, synthetic construct
  source 1..60: mol_type = other DNA
ggcggtagcggcggcggtagcggcggaggcagcggtggcggcagcggcggcggtagcggc

SEQ ID NO: 13 - DNA, 45 bp, synthetic construct
  source 1..45: mol_type = other DNA
accggtggtggtcccgggggtggtgcggccgcaggcagcggaagc

SEQ ID NO: 14 - DNA, 96 bp, synthetic construct
  source 1..96: mol_type = other DNA
tctggaggatctagcggaggatcctctggaagcgagacaccaggcacaagcgagtccgccacaccagagagctccggcggctcctccggaggatcc

SEQ ID NO: 15 - protein, 9 aa, unidentified
LAGLIDADG

SEQ ID NO: 16 - protein, 7 aa, Simian virus 40
PKKKRKV

SEQ ID NO: 17 - protein, 16 aa, unidentified
KRPAATKKAGQAKKKK

SEQ ID NO: 18 - protein, 9 aa, unidentified
PAAKRVKLD

SEQ ID NO: 19 - protein, 11 aa, unidentified
RQRRNELKRSP

SEQ ID NO: 20 - protein, 38 aa, Homo sapiens
NQSSNFGPMKGGNFGGRSSGPYGGGGQYFAKPRNQGGY

SEQ ID NO: 21 - protein, 42 aa, unidentified
RMRIZFKNKGKDTAELRRRRVEVSVELRKAKKDEQILKRRNV

SEQ ID NO: 22 - protein, 8 aa, unidentified
VSRKRPRP

SEQ ID NO: 23 - protein, 8 aa, unidentified
PPKKARED

SEQ ID NO: 24 - protein, 8 aa, Homo sapiens
PQPKKKPL

SEQ ID NO: 25 - protein, 12 aa, Mus sp.
SALIKKKKKMAP

SEQ ID NO: 26 - protein, 5 aa, Influenza A virus
DRLRR

SEQ ID NO: 27 - protein, 7 aa, Influenza A virus
PKQKKRK

SEQ ID NO: 28 - protein, 10 aa, Hepatitis Delta virus
RKLKKKIKKL

SEQ ID NO: 29 - protein, 10 aa, Mus sp.
REKKKFLKRR

SEQ ID NO: 30 - protein, 20 aa, Homo sapiens
KRKGDEVDGVDEVAKKKSKK

SEQ ID NO: 31 - protein, 17 aa, Homo sapiens
RKCLQAGMNLEARKTKK

SEQ ID NO: 32 - protein, 21 aa, unidentified
GKRKLITSEEERSPAKRGRKS

SEQ ID NO: 33 - protein, 6 aa, unidentified
KRKRRP

SEQ ID NO: 34 - protein, 20 aa, unidentified
KRKGSPCDTLASSTEKRRRE

SEQ ID NO: 35 - protein, 12 aa, unidentified
KRNFRSALNRKE

SEQ ID NO: 36 - DNA, 20 bp, synthetic construct
  source 1..20: mol_type = other DNA
tctgaggagctagagacttg

SEQ ID NO: 37 - DNA, 20 bp, synthetic construct
  source 1..20: mol_type = other DNA
agcaaataggcttagtgtgc

SEQ ID NO: 38 - DNA, 20 bp, synthetic construct
  source 1..20: mol_type = other DNA
ggctaaataatgaatgtccc

SEQ ID NO: 39 - DNA, 20 bp, synthetic construct
  source 1..20: mol_type = other DNA
tcccttcctagaattggcct

SEQ ID NO: 40 - DNA, 20 bp, synthetic construct
  source 1..20: mol_type = other DNA
ttcccttcctagaattggcc

SEQ ID NO: 41 - DNA, 20 bp, synthetic construct
  source 1..20: mol_type = other DNA
gaatgtcccaggccaattct

SEQ ID NO: 42 - DNA, 20 bp, synthetic construct
  source 1..20: mol_type = other DNA
cccacttcccttcctagaat

SEQ ID NO: 43 - DNA, 20 bp, synthetic construct
  source 1..20: mol_type = other DNA
cctggtaccaggaaggcaat

SEQ ID NO: 44 - DNA, 20 bp, synthetic construct
  source 1..20: mol_type = other DNA
tcctggtaccaggaaggcaa

SEQ ID NO: 45 - DNA, 20 bp, synthetic construct
  source 1..20: mol_type = other DNA
gcatcatcctggtaccagga

SEQ ID NO: 46 - DNA, 20 bp, synthetic construct
  source 1..20: mol_type = other DNA
cattgcatcatcctggtacc

SEQ ID NO: 47 - DNA, 20 bp, synthetic construct
  source 1..20: mol_type = other DNA
ctccaagcattgcatcatcc

SEQ ID NO: 48 - DNA, 20 bp, synthetic construct
  source 1..20: mol_type = other DNA
taccaggatgatgcaatgct

SEQ ID NO: 49 - DNA, 20 bp, synthetic construct
  source 1..20: mol_type = other DNA
gggtgtgccctgagaaggtg

SEQ ID NO: 50 - DNA, 20 bp, synthetic construct
  source 1..20: mol_type = other DNA
agggtgtgccctgagaaggt

SEQ ID NO: 51 - DNA, 20 bp, synthetic construct
  source 1..20: mol_type = other DNA
tcacagggtgtgccctgaga

SEQ ID NO: 52 - DNA, 20 bp, synthetic construct
  source 1..20: mol_type = other DNA
ggcacaccctgtgatcttgt

SEQ ID NO: 53 - DNA, 20 bp, synthetic construct
  source 1..20: mol_type = other DNA
agcacacaagatgcacaccc

SEQ ID NO: 54 - DNA, 20 bp, synthetic construct
  source 1..20: mol_type = other DNA
tgtgcttggtcggcactgat

SEQ ID NO: 55 - DNA, 20 bp, synthetic construct
  source 1..20: mol_type = other DNA
gtgcttggtcggcactgata

SEQ ID NO: 56 - DNA, 20 bp, synthetic construct
  source 1..20: mol_type = other DNA
tgcttggtcggcactgatag

SEQ ID NO: 57 - DNA, 20 bp, synthetic construct
  source 1..20: mol_type = other DNA
gggtcgcggtagggagttgt

SEQ ID NO: 58 - DNA, 20 bp, synthetic construct
  source 1..20: mol_type = other DNA
gccaacagtgataaccagca

SEQ ID NO: 59 - DNA, 20 bp, synthetic construct
  source 1..20: mol_type = other DNA
tgccaacagtgataaccagc

SEQ ID NO: 60 - DNA, 20 bp, synthetic construct
  source 1..20: mol_type = other DNA
gccctgctggttatcactgt

SEQ ID NO: 61 - DNA, 20 bp, synthetic construct
  source 1..20: mol_type = other DNA
agcagccctgggcacagaag

SEQ ID NO: 62 - DNA, 20 bp, synthetic construct
  source 1..20: mol_type = other DNA
cctctatgtagacgggtgtg

SEQ ID NO: 63 - DNA, 20 bp, synthetic construct
  source 1..20: mol_type = other DNA
ggaagggcctctatgtagac

SEQ ID NO: 64 - DNA, 20 bp, synthetic construct
  source 1..20: mol_type = other DNA
ggaggtgtggaggggataac

SEQ ID NO: 65 - DNA, 20 bp, synthetic construct
  source 1..20: mol_type = other DNA
ctggcagaccctcaagagca

SEQ ID NO: 66 - DNA, 20 bp, synthetic construct
  source 1..20: mol_type = other DNA
cccatggaggtggggagatg

SEQ ID NO: 67 - DNA, 20 bp, synthetic construct
  source 1..20: mol_type = other DNA
gtcatcctcggccaatgaag

SEQ ID NO: 68 - DNA, 20 bp, synthetic construct
  source 1..20: mol_type = other DNA
aagtgagccaggtgatagaa

SEQ ID NO: 69 - DNA, 20 bp, synthetic construct
  source 1..20: mol_type = other DNA
tgaaaccaagcttcctctgc

SEQ ID NO: 70 - DNA, 20 bp, synthetic construct
  source 1..20: mol_type = other DNA
agggagaaatgagacaaaag

SEQ ID NO: 71 - DNA, 20 bp, synthetic construct
  source 1..20: mol_type = other DNA
aagaggccactgagtccttt

SEQ ID NO: 72 - DNA, 20 bp, synthetic construct
  source 1..20: mol_type = other DNA
ctaacagttgcttttatcac

SEQ ID NO: 73 - DNA, 20 bp, synthetic construct
  source 1..20: mol_type = other DNA
ttgcttttatcacaggctcc

SEQ ID NO: 74 - DNA, 20 bp, synthetic construct
  source 1..20: mol_type = other DNA
ttttatcacaggctccagga

SEQ ID NO: 75 - DNA, 20 bp, synthetic construct
  source 1..20: mol_type = other DNA
tttatcacaggctccaggaa

SEQ ID NO: 76 - DNA, 20 bp, synthetic construct
  source 1..20: mol_type = other DNA
atcagaggccaaacccttcc

SEQ ID NO: 77 - DNA, 20 bp, synthetic construct
  source 1..20: mol_type = other DNA
cacaggctccaggaagggtt

SEQ ID NO: 78 - DNA, 20 bp, synthetic construct
  source 1..20: mol_type = other DNA
cacgcccccaccctaatcag

SEQ ID NO: 79 - DNA, 20 bp, synthetic construct
  source 1..20: mol_type = other DNA
gaagggtttggcctctgatt

SEQ ID NO: 80 - DNA, 20 bp, synthetic construct
  source 1..20: mol_type = other DNA
aagggtttggcctctgatta

SEQ ID NO: 81 - DNA, 20 bp, synthetic construct
  source 1..20: mol_type = other DNA
ggtttggcctctgattaggg

SEQ ID NO: 82 - DNA, 20 bp, synthetic construct
  source 1..20: mol_type = other DNA
gtttggcctctgattagggt

SEQ ID NO: 83 - DNA, 20 bp, synthetic construct
  source 1..20: mol_type = other DNA
tttggcctctgattagggtg

SEQ ID NO: 84 - DNA, 20 bp, synthetic construct
  source 1..20: mol_type = other DNA
ttggcctctgattagggtgg

SEQ ID NO: 85 - DNA, 20 bp, synthetic construct
  source 1..20: mol_type = other DNA
tctgattagggtgggggcgt

SEQ ID NO: 86 - DNA, 20 bp, synthetic construct
  source 1..20: mol_type = other DNA
attagggtgggggcgtgggt

SEQ ID NO: 87 - DNA, 20 bp, synthetic construct
  source 1..20: mol_type = other DNA
tgggtggggtagaagaggac

SEQ ID NO: 88 - DNA, 20 bp, synthetic construct
  source 1..20: mol_type = other DNA
gcaaacggccaccgatggag

SEQ ID NO: 89 - DNA, 20 bp, synthetic construct
  source 1..20: mol_type = other DNA
cctgggcaaacggccaccga

SEQ ID NO: 90 - DNA, 20 bp, synthetic construct
  source 1..20: mol_type = other DNA
aagaggcccccctgggcaaa

SEQ ID NO: 91 - DNA, 20 bp, synthetic construct
  source 1..20: mol_type = other DNA
ccatcggtggccgtttgccc

SEQ ID NO: 92 - DNA, 20 bp, synthetic construct
  source 1..20: mol_type = other DNA
catcggtggccgtttgccca

SEQ ID NO: 93 - DNA, 20 bp, synthetic construct
  source 1..20: mol_type = other DNA
atcggtggccgtttgcccag

SEQ ID NO: 94 - DNA, 20 bp, synthetic construct
  source 1..20: mol_type = other DNA
tcggtggccgtttgcccagg

SEQ ID NO: 95 - DNA, 20 bp, synthetic construct
  source 1..20: mol_type = other DNA
cggtggccgtttgcccaggg

SEQ ID NO: 96 - DNA, 20 bp, synthetic construct
  source 1..20: mol_type = other DNA
cttccgaaagaggcccccct

SEQ ID NO: 97 - DNA, 20 bp, synthetic construct
  source 1..20: mol_type = other DNA
ccttccgaaagaggcccccc

SEQ ID NO: 98 - DNA, 20 bp, synthetic construct
  source 1..20: mol_type = other DNA
tcagggggaggcaagtcagt

SEQ ID NO: 99 - DNA, 20 bp, synthetic construct
  source 1..20: mol_type = other DNA
agggaaaagggagaggaaaa

SEQ ID NO: 100 - DNA, 20 bp, synthetic construct
  source 1..20: mol_type = other DNA
tgtaactaataaataccagg

SEQ ID NO: 101 - DNA, 20 bp, synthetic construct
  source 1..20: mol_type = other DNA
ccagctgaagaaagaacatt

SEQ ID NO: 102 - DNA, 20 bp, synthetic construct
  source 1..20: mol_type = other DNA
ccatctccctaatctccaat

SEQ ID NO: 103 - DNA, 20 bp, synthetic construct
  source 1..20: mol_type = other DNA
tggggagagaagagtggaaa

SEQ ID NO: 104 - DNA, 20 bp, synthetic construct
  source 1..20: mol_type = other DNA
ggagtatggggagagaagag

SEQ ID NO: 105 - DNA, 20 bp, synthetic construct
  source 1..20: mol_type = other DNA
acaacctccttgtttacaga

SEQ ID NO: 106 - DNA, 20 bp, synthetic construct
  source 1..20: mol_type = other DNA
gagatttactcttgttgccc

SEQ ID NO: 107 - DNA, 20 bp, synthetic construct
  source 1..20: mol_type = other DNA
ttgcccgggctggaatgcaa

SEQ ID NO: 108 - DNA, 20 bp, synthetic construct
  source 1..20: mol_type = other DNA
ggagatcgcttgaacctggg

SEQ ID NO: 109 - DNA, 20 bp, synthetic construct
  source 1..20: mol_type = other DNA
ctcagctactcgggaggctg

SEQ ID NO: 110 - DNA, 20 bp, synthetic construct
  source 1..20: mol_type = other DNA
tgtaatctcagctactcggg

SEQ ID NO: 111 - DNA, 20 bp, synthetic construct
  source 1..20: mol_type = other DNA
gcctgtaatctcagctactc

SEQ ID NO: 112 - DNA, 20 bp, synthetic construct
  source 1..20: mol_type = other DNA
tgcctgtaatctcagctact

SEQ ID NO: 113 - DNA, 20 bp, synthetic construct
  source 1..20: mol_type = other DNA
caggcatgtattaccatgcc

SEQ ID NO: 114 - DNA, 20 bp, synthetic construct
  source 1..20: mol_type = other DNA
caggaggatcacctgaggtc

SEQ ID NO: 115 - DNA, 20 bp, synthetic construct
  source 1..20: mol_type = other DNA
ctcaggtgatcctcctgccc

SEQ ID NO: 116 - DNA, 20 bp, synthetic construct
  source 1..20: mol_type = other DNA
cccagcactttgggaggccg

SEQ ID NO: 117 - DNA, 20 bp, synthetic construct
  source 1..20: mol_type = other DNA
tcccagcactttgggaggcc

SEQ ID NO: 118 - DNA, 20 bp, synthetic construct
  source 1..20: mol_type = other DNA
atcccagcactttgggaggc

SEQ ID NO: 119 - DNA, 20 bp, synthetic construct
  source 1..20: mol_type = other DNA
acctgtaatcccagcacttt

SEQ ID NO: 120 - DNA, 20 bp, synthetic construct
  source 1..20: mol_type = other DNA
gccccggcctcccaaagtgc

SEQ ID NO: 121 - DNA, 20 bp, synthetic construct
  source 1..20: mol_type = other DNA
ccccggcctcccaaagtgct

SEQ ID NO: 122 - DNA, 20 bp, synthetic construct
  source 1..20: mol_type = other DNA
atttgctcttctccagggtg

SEQ ID NO: 123 - DNA, 20 bp, synthetic construct
  source 1..20: mol_type = other DNA
taaacagccaccccacaccc

SEQ ID NO: 124 - DNA, 20 bp, synthetic construct
  source 1..20: mol_type = other DNA
tttgctcttctccagggtgt

SEQ ID NO: 125 - DNA, 20 bp, synthetic construct
  source 1..20: mol_type = other DNA
ctcttctccagggtgtgggg

SEQ ID NO: 126 - DNA, 20 bp, synthetic construct
  source 1..20: mol_type = other DNA
tgtggggtggctgtttaaag

SEQ ID NO: 127 - DNA, 20 bp, synthetic construct
  source 1..20: mol_type = other DNA
gggtggctgtttaaagaggg

SEQ ID NO: 128 - DNA, 20 bp, synthetic construct
  source 1..20: mol_type = other DNA
agttcaagtagatatcagaa

SEQ ID NO: 129 - DNA, 20 bp, synthetic construct
  source 1..20: mol_type = other DNA
tatcagaagggaactgtttg

SEQ ID NO: 130 - DNA, 20 bp, synthetic construct
  source 1..20: mol_type = other DNA
aagaatggcttcaagaggct

SEQ ID NO: 131 - DNA, 20 bp, synthetic construct
  source 1..20: mol_type = other DNA
tctgtaagaatggcttcaag

SEQ ID NO: 132 - DNA, 20 bp, synthetic construct
  source 1..20: mol_type = other DNA
acagatgatgaaccagacca

SEQ ID NO: 133 - DNA, 20 bp, synthetic construct
  source 1..20: mol_type = other DNA
tgaaccagaccacggcccgt

SEQ ID NO: 134 - DNA, 20 bp, synthetic construct
  source 1..20: mol_type = other DNA
gaaccagaccacggcccgtt

SEQ ID NO: 135 - DNA, 20 bp, synthetic construct
  source 1..20: mol_type = other DNA
ggcccgttgggagctccaga

SEQ ID NO: 136 - DNA, 20 bp, synthetic construct
  source 1..20: mol_type = other DNA
gcccgttgggagctccagaa

SEQ ID NO: 137 - DNA, 20 bp, synthetic construct
  source 1..20: mol_type = other DNA
cccgttgggagctccagaag

SEQ ID NO: 138 - DNA, 20 bp, synthetic construct
  source 1..20: mol_type = other DNA
ctggagctcccaacgggccg

SEQ ID NO: 139 - DNA, 20 bp, synthetic construct
  source 1..20: mol_type = other DNA
ccccttctggagctcccaac

SEQ ID NO: 140 - DNA, 20 bp, synthetic construct
  source 1..20: mol_type = other DNA
tccccttctggagctcccaa

SEQ ID NO: 141 - DNA, 20 bp, synthetic construct
  source 1..20: mol_type = other DNA
gatcatgacctcctcacctg

SEQ ID NO: 142 - DNA, 20 bp, synthetic construct
  source 1..20: mol_type = other DNA
atcatgacctcctcacctgt

SEQ ID NO: 143 - DNA, 20 bp, synthetic construct
  source 1..20: mol_type = other DNA
aggaggtcatgatccccttc

SEQ ID NO: 144 - DNA, 20 bp, synthetic construct
  source 1..20: mol_type = other DNA
ggcactgcccacaggtgagg

SEQ ID NO: 145 - DNA, 20 bp, synthetic construct
  source 1..20: mol_type = other DNA
gtgccagatgaacttcccat

SEQ ID NO: 146 - DNA, 20 bp, synthetic construct
  source 1..20: mol_type = other DNA
tgccagatgaacttcccatt

SEQ ID NO: 147 - DNA, 20 bp, synthetic construct
  source 1..20: mol_type = other DNA
gccagatgaacttcccattg

SEQ ID NO: 148 - DNA, 20 bp, synthetic construct
  source 1..20: mol_type = other DNA
tctggcactgcccacaggtg

SEQ ID NO: 149 - DNA, 20 bp, synthetic construct
  source 1..20: mol_type = other DNA
ccagatgaacttcccattgg

SEQ ID NO: 150 - DNA, 20 bp, synthetic construct
  source 1..20: mol_type = other DNA
gttcatctggcactgcccac

SEQ ID NO: 151 - DNA, 20 bp, synthetic construct
  source 1..20: mol_type = other DNA
cccccaatgggaagttcatc

SEQ ID NO: 152 - DNA, 20 bp, synthetic construct
  source 1..20: mol_type = other DNA
aaataagaatgtcccccaat

SEQ ID NO: 153 - DNA, 20 bp, synthetic construct
  source 1..20: mol_type = other DNA
aaaataagaatgtcccccaa

SEQ ID NO: 154 - DNA, 20 bp, synthetic construct
  source 1..20: mol_type = other DNA
cacaaacggaaacaatgcaa

SEQ ID NO: 155 - DNA, 20 bp, synthetic construct
  source 1..20: mol_type = other DNA
cctctgcttagaaaaagctg

SEQ ID NO: 156 - DNA, 20 bp, synthetic construct
  source 1..20: mol_type = other DNA
ccacagctttttctaagcag

SEQ ID NO: 157 - DNA, 20 bp, synthetic construct
  source 1..20: mol_type = other DNA
tcgattggtgaaggggaagg

SEQ ID NO: 158 - DNA, 20 bp, synthetic construct
  source 1..20: mol_type = other DNA
atctcgattggtgaagggga

SEQ ID NO: 159 - DNA, 20 bp, synthetic construct
  source 1..20: mol_type = other DNA
tttcatctcgattggtgaag

SEQ ID NO: 160 - DNA, 20 bp, synthetic construct
  source 1..20: mol_type = other DNA
gaaaaaagcatccaatcccg

SEQ ID NO: 161 - DNA, 20 bp, synthetic construct
  source 1..20: mol_type = other DNA
aaaagcatccaatcccgtgg

SEQ ID NO: 162 - DNA, 20 bp, synthetic construct
  source 1..20: mol_type = other DNA
gcatccaatcccgtggaggt

SEQ ID NO: 163 - DNA, 20 bp, synthetic construct
  source 1..20: mol_type = other DNA
tcccgtggaggttggcatcc

SEQ ID NO: 164 - DNA, 20 bp, synthetic construct
  source 1..20: mol_type = other DNA
tggcatccaggtcacgccag

SEQ ID NO: 165 - DNA, 20 bp, synthetic construct
  source 1..20: mol_type = other DNA
gatgccaacctccacgggat

SEQ ID NO: 166 - DNA, 20 bp, synthetic construct
  source 1..20: mol_type = other DNA
acctggatgccaacctccac

SEQ ID NO: 167 - DNA, 20 bp, synthetic construct
  source 1..20: mol_type = other DNA
gacctggatgccaacctcca

SEQ ID NO: 168 - DNA, 20 bp, synthetic construct
  source 1..20: mol_type = other DNA
cgtcatcctctggcgtgacc

SEQ ID NO: 169 - DNA, 20 bp, synthetic construct
  source 1..20: mol_type = other DNA
gataaacaatcgtcatcctc

SEQ ID NO: 170 - DNA, 20 bp, synthetic construct
  source 1..20: mol_type = other DNA
ctgctatgtgttcctgtttg

SEQ ID NO: 171 - DNA, 20 bp, Homo sapiens
  source 1..20: mol_type = genomic DNA
tggaaaggagaacggcccgg

SEQ ID NO: 172 - DNA, 20 bp, Homo sapiens
  source 1..20: mol_type = genomic DNA
tgaacaccctcgttaaaggc

SEQ ID NO: 173 - DNA, 20 bp, Homo sapiens
  source 1..20: mol_type = genomic DNA
aacactagcccacatgccaa

SEQ ID NO: 174 - DNA, 20 bp, Homo sapiens
  source 1..20: mol_type = genomic DNA
gcccacagaggcacggttaa

SEQ ID NO: 175 - DNA, 20 bp, Homo sapiens
  source 1..20: mol_type = genomic DNA
aggcacggttaatggtggcg

SEQ ID NO: 176 - DNA, 20 bp, Homo sapiens
  source 1..20: mol_type = genomic DNA
cacaggaagccatggtcctt

SEQ ID NO: 177 - DNA, 20 bp, Homo sapiens
  source 1..20: mol_type = genomic DNA
gcactgacgtaggtagtgac

SEQ ID NO: 178 - DNA, 20 bp, Homo sapiens
  source 1..20: mol_type = genomic DNA
ataggatatggcactgacgt

SEQ ID NO: 179 - DNA, 20 bp, Homo sapiens
  source 1..20: mol_type = genomic DNA
cattatcttctctggtctcg

SEQ ID NO: 180 - DNA, 20 bp, Homo sapiens
  source 1..20: mol_type = genomic DNA
atactggggaacacattgta

SEQ ID NO: 181 - DNA, 20 bp, Homo sapiens
  source 1..20: mol_type = genomic DNA
tgagcacattcttacgccta

SEQ ID NO: 182 - DNA, 20 bp, Homo sapiens
  source 1..20: mol_type = genomic DNA
ctaggcgtaagaatgtgctc

SEQ ID NO: 183 - DNA, 20 bp, Homo sapiens
  source 1..20: mol_type = genomic DNA
gaaccccctataaactagtc

SEQ ID NO: 184 - DNA, 20 bp, Homo sapiens
  source 1..20: mol_type = genomic DNA
ggcaaaccagactagtttat

SEQ ID NO: 185 - DNA, 20 bp, Homo sapiens
  source 1..20: mol_type = genomic DNA
caggggagaactcggcatga

SEQ ID NO: 186 - DNA, 20 bp, Homo sapiens
  source 1..20: mol_type = genomic DNA
gatggagttggttgaccgta

SEQ ID NO: 187 - DNA, 20 bp, Homo sapiens
  source 1..20: mol_type = genomic DNA
ggtaggacccaacactacgc

SEQ ID NO: 188 - DNA, 20 bp, Homo sapiens
  source 1..20: mol_type = genomic DNA
atgcctagggtgttttgacg

SEQ ID NO: 189 - DNA, 20 bp, Homo sapiens
  source 1..20: mol_type = genomic DNA
tgaaccagaccacggcccgt

SEQ ID NO: 190 - DNA, 20 bp, Homo sapiens
  source 1..20: mol_type = genomic DNA
gcatccaatcccgtggaggt

SEQ ID NO: 191 - DNA, 20 bp, Homo sapiens
  source 1..20: mol_type = genomic DNA
cactggcttcctgttcttgt

SEQ ID NO: 192 - DNA, 20 bp, Homo sapiens
  source 1..20: mol_type = genomic DNA
aaggttttcaaggcaaataa

SEQ ID NO: 193 - DNA, 20 bp, Homo sapiens
  source 1..20: mol_type = genomic DNA
gtaatggagcccgcatgctg

SEQ ID NO: 194 - DNA, 20 bp, Homo sapiens
  source 1..20: mol_type = genomic DNA
gccagtgtacaggcaagtac

SEQ ID NO: 195 - DNA, 20 bp, Homo sapiens
  source 1..20: mol_type = genomic DNA
tcgctgccttcagttctgct

SEQ ID NO: 196 - DNA, 20 bp, Homo sapiens
  source 1..20: mol_type = genomic DNA
ttatggaactcaggaactgc

SEQ ID NO: 197 - DNA, 20 bp, Homo sapiens
  source 1..20: mol_type = genomic DNA
gatgcctttttcatctcgat

SEQ ID NO: 198 - DNA, 20 bp, Homo sapiens
  source 1..20: mol_type = genomic DNA
attccttgagtgtcatatat

SEQ ID NO: 199 - DNA, 20 bp, Homo sapiens
  source 1..20: mol_type = genomic DNA
tctggaatcactatgtatat

SEQ ID NO: 200 - DNA, 23 bp, synthetic construct
  source 1..23: mol_type = other DNA
  misc_difference 9: note = a, g, t, or c
  misc_difference 10: note = a, g, t, or c
  misc_difference 11: note = a, g, t, or c
  misc_difference 12: note = a, g, t, or c
  misc_difference 13: note = a, g, t, or c
  misc_difference 14: note = a, g, t, or c
  misc_difference 15: note = a, g, t, or c
  misc_difference 16: note = a, g, t, or c
  misc_difference 17: note = a, g, t, or c
  misc_difference 18: note = a, g, t, or c
  misc_difference 19: note = a, g, t, or c
  misc_difference 20: note = a, g, t, or c
  misc_difference 21: note = a, g, t, or c
mmmmmmmmnnnnnnnnnnnnngg

SEQ ID NO: 201 - DNA, 23 bp, synthetic construct
  source 1..23: mol_type = other DNA
  misc_difference 10: note = a, g, t, or c
  misc_difference 11: note = a, g, t, or c
  misc_difference 12: note = a, g, t, or c
  misc_difference 13: note = a, g, t, or c
  misc_difference 14: note = a, g, t, or c
  misc_difference 15: note = a, g, t, or c
  misc_difference 16: note = a, g, t, or c
  misc_difference 17: note = a, g, t, or c
  misc_difference 18: note = a, g, t, or c
  misc_difference 19: note = a, g, t, or c
  misc_difference 20: note = a, g, t, or c
  misc_difference 21: note = a, g, t, or c
mmmmmmmmmnnnnnnnnnnnngg

SEQ ID NO: 202 - DNA, 54 bp, synthetic construct
  source 1..54: mol_type = other DNA
gaatgtcccaggccaattctaggaagggaagtgggtatggggcagcccattgcc

SEQ ID NO: 203 - DNA, 54 bp, synthetic construct
  source 1..54: mol_type = other DNA
ggcaatgggctgccccatacccacttcccttcctagaattggcctgggacattc

SEQ ID NO: 204 - DNA, 54 bp, synthetic construct
  source 1..54: mol_type = other DNA
ttcctggtaccaggatgatgcaatgcttggaggctgtgagctccccaccttctc

SEQ ID NO: 205 - DNA, 54 bp, synthetic construct
  source 1..54: mol_type = other DNA
gagagaaggtggagctcacagcctccaagcattgcatcatcctggtaccaggaa

SEQ ID NO: 206 - DNA, 54 bp, synthetic construct
  source 1..54: mol_type = other DNA
agggcacaccctgtgatcttgtgggacccctctgtccagcccagcctgggtgtg

SEQ ID NO: 207 - DNA, 54 bp, synthetic construct
  source 1..54: mol_type = other DNA
cacacccaggctgggctggacagaggggtcccacaacatcacagggtgtgccct

SEQ ID NO: 208 - DNA, 54 bp, synthetic construct
  source 1..54: mol_type = other DNA
catcttgtgtgcttggtcggcactgataggggtcgcggtagggagttgtcggca

SEQ ID NO: 209 - DNA, 54 bp, synthetic construct
  source 1..54: mol_type = other DNA
tgccgacaactcccgaccgcgacccctatcagtgccgaccaagcacacaagatg

SEQ ID NO: 210 - DNA, 23 bp, synthetic construct
  source 1..23: mol_type = other DNA
tgcttggtcggcactgatagggg

SEQ ID NO: 211 - DNA, 28 bp, synthetic construct
  source 1..28: mol_type = other DNA
ctaacagttgcttttatcacaggctcca

SEQ ID NO: 212 - DNA, 28 bp, synthetic construct
  source 1..28: mol_type = other DNA
tggagcctgtgataaaagcaactgttag

SEQ ID NO: 213 - DNA, 23 bp, synthetic construct
  source 1..23: mol_type = other DNA
cccctatcagtgccgaccaagca

SEQ ID NO: 214 - DNA, 34 bp, synthetic construct
  source 1..34: mol_type = other DNA
tgcttggtcggcactgaataaaagcaactgttag

SEQ ID NO: 215 - DNA, 17 bp, synthetic construct
  source 1..17: mol_type = other DNA
cccctacacaggctcca

SEQ ID NO: 216 - DNA, 17 bp, synthetic construct
  source 1..17: mol_type = other DNA
tggagcctgtgtagggg

SEQ ID NO: 217 - DNA, 34 bp, synthetic construct
  source 1..34: mol_type = other DNA
ctaacagttgcttttattcagtgccgaccaagca

SEQ ID NO: 218 - DNA, 33 bp, synthetic construct
  source 1..33: mol_type = other DNA
tgcttggtcggcactgataaaagcaactgttag

SEQ ID NO: 219 - DNA, 18 bp, synthetic construct
  source 1..18: mol_type = other DNA
cccctatcacaggctcca

SEQ ID NO: 220 - DNA, 18 bp, synthetic construct
  source 1..18: mol_type = other DNA
tggagcctgtgatagggg

SEQ ID NO: 221 - DNA, 33 bp, synthetic construct
  source 1..33: mol_type = other DNA
ctaacagttgcttttatcagtgccgaccaagca

SEQ ID NO: 222 - DNA, 23 bp, synthetic construct
  source 1..23: mol_type = other DNA
ttgcttttatcacaggctccagg

SEQ ID NO: 223 - DNA, 23 bp, synthetic construct
  source 1..23: mol_type = other DNA
cctggagcctgtgataaaagcaa

SEQ ID NO: 224 - DNA, 34 bp, synthetic construct
  source 1..34: mol_type = other DNA
tgcttggtcggcactgagcctgtgataaaagcaa

SEQ ID NO: 225 - DNA, 12 bp, synthetic construct
  source 1..12: mol_type = other DNA
cccctatccagg

SEQ ID NO: 226 - DNA, 12 bp, synthetic construct
  source 1..12: mol_type = other DNA
cctggatagggg

SEQ ID NO: 227 - DNA, 34 bp, synthetic construct
  source 1..34: mol_type = other DNA
ttgcttttatcacaggctcagtgccgaccaagca

SEQ ID NO: 228 - DNA, 33 bp, synthetic construct
  source 1..33: mol_type = other DNA
tgcttggtcggcactggcctgtgataaaagcaa

SEQ ID NO: 229 - DNA, 13 bp, synthetic construct
  source 1..13: mol_type = other DNA
cccctattccagg

SEQ ID NO: 230 - DNA, 13 bp, synthetic construct
  source 1..13: mol_type = other DNA
gaaggaatagggg

SEQ ID NO: 231 - DNA, 33 bp, synthetic construct
  source 1..33: mol_type = other DNA
ttgcttttatcacaggccagtgccgaccaagca